SEQUENCE LISTING (27 sequences)

SEQ ID NO: 1 - protein, 328 aa, Homo sapiens
  MISC_FEATURE: Wild-type human IL-12p40 precursor
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 335 aa, Mus musculus
  MISC_FEATURE: Wild-type mouse IL-12p40 precursor
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 3 - protein, 328 aa, Artificial sequence
  Synthetic construct
  MISC_FEATURE: human IL-12p40 variant E81A
MCHQQLVISWFSLVFLASPLVAIWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 328 aa, Artificial sequence
  Synthetic construct
  MISC_FEATURE: human IL-12p40 variant F82A
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 328 aa, Artificial sequence
  Synthetic construct
  MISC_FEATURE: human IL-12p40 variant P39A/D40A/E81A/F82A
MCHQQLVISWFSLVFLASPLVAIWELKKDVYVVELDWYAAAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 328 aa, Artificial sequence
  Synthetic construct
  MISC_FEATURE: human IL-12p40 variant K106A
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 328 aa, Artificial sequence
  Synthetic construct
  MISC_FEATURE: human IL-12p40 variant K217A
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHALKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 8 - protein, 328 aa, Artificial sequence
  Synthetic construct
  MISC_FEATURE: human IL-12p40 variant K219A
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLAYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 335 aa, Artificial sequence
  Synthetic construct
  MISC_FEATURE: mouse IL-12p40 variant E81A/F82A
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 10 - protein, 335 aa, Artificial sequence
  Synthetic construct
  MISC_FEATURE: mouse IL-12p40 variant E81A/F82A/K106A
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 11 - protein, 335 aa, Artificial sequence
  Synthetic construct
  MISC_FEATURE: mouse IL-12p40 variant E81A/F82A/K106A/K217A
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHAKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNAYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 15 aa, Artificial sequence
  Synthetic construct
  MISC_FEATURE: AviTag
GLNDIFEAQKIEWHE

SEQ ID NO: 13 - protein, 328 aa, Artificial sequence
  Synthetic construct
  MISC_FEATURE: human IL-12p40 variant E81A/F82A
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 328 aa, Artificial sequence
  Synthetic construct
  MISC_FEATURE: human IL-12p40 variant E81A/F82A/K106A
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - protein, 328 aa, Artificial sequence
  Synthetic construct
  MISC_FEATURE: human IL-12p40 variant W37A/E81A/F82A
MCHQQLVISWFSLVFLASPLVAIWELKKDVYVVELDAYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 16 - protein, 328 aa, Artificial sequence
  Synthetic construct
  MISC_FEATURE: human IL-12p40 variant E81A/F82A/K106A/E108A/ D115A
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKADGIWSTAILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 17 - protein, 335 aa, Artificial sequence
  Synthetic construct
  MISC_FEATURE: mouse IL-12p40 variant E81F/F82A
MCPQKLTISWFAIVLLVSPLMAMWELEKDVYVVEVDWTPDAPGETVNLTCDTPEEDDITWTSDQRHGVIGSGKTLTITVKFALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 18 - protein, 335 aa, Artificial sequence
  Synthetic construct
  MISC_FEATURE: mouse IL-12p40 variant E81K/F82A
MCPQKLTISWFAIVLLVSPLMAMWELEKDVYVVEVDWTPDAPGETVNLTCDTPEEDDITWTSDQRHGVIGSGKTLTITVKKALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 19 - protein, 335 aa, Artificial sequence
  Synthetic construct
  MISC_FEATURE: mouse IL-12p40 variant E81L/F82A
MCPQKLTISWFAIVLLVSPLMAMWELEKDVYVVEVDWTPDAPGETVNLTCDTPEEDDITWTSDQRHGVIGSGKTLTITVKLALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 20 - protein, 335 aa, Artificial sequence
  Synthetic construct
  misc_feature: mouse IL-12p40 variant E81H/F82A
MCPQKLTISWFAIVLLVSPLMAMWELEKDVYVVEVDWTPDAPGETVNLTCDTPEEDDITWTSDQRHGVIGSGKTLTITVKHALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 21 - protein, 335 aa, Artificial sequence
  Synthetic construct
  MISC_FEATURE: mouse IL-12p40 variant E81S/F82A
MCPQKLTISWFAIVLLVSPLMAMWELEKDVYVVEVDWTPDAPGETVNLTCDTPEEDDITWTSDQRHGVIGSGKTLTITVKSALDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 22 - protein, 335 aa, Artificial sequence
  Synthetic construct
  MISC_FEATURE: mouse IL-12p40 variant E81A/F82A/K106N
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHNKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 23 - protein, 335 aa, Artificial sequence
  Synthetic construct
  MISC_FEATURE: mouse IL-12p40 variant E81A/F82A/K106Q
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHQKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 24 - protein, 335 aa, Artificial sequence
  Synthetic construct
  MISC_FEATURE: mouse IL-12p40 variant E81A/F82A/K106T
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHTKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 25 - protein, 335 aa, Artificial sequence
  Synthetic construct
  MISC_FEATURE: mouse IL-12p40 variant E81A/F82A/K106R
MCPQKLTISWFAIVLLVSPLMAMWELEKDVYVVEVDWTPDAPGETVNLTCDTPEEDDITWTSDQRHGVIGSGKTLTITVKAALDAGQYTCHKGGETLSHSHLLLHRKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 26 - protein, 306 aa, Homo sapiens
  MISC_FEATURE: Mature wild-type human IL-12p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 27 - protein, 313 aa, Mus musculus
  MISC_FEATURE: Mature wild-type murine IL-12p40
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

(19) United States
(12) Patent Application Publication
(10) Pub. No.: US 2023/0220031 A1
(43) Pub. Date: Jul. 13, 2023

(54) ENGINEERED IL-12 AND IL-23 POLYPEPTIDES AND USES THEREOF

(71) Applicant: The Board of Trustees of the Leland Stanford Junior University, Stanford, CA (US)

(72) Inventors: Kenan Christopher GARCIA, Palo Alto, CA (US); Caleb R. GLASSMAN, Palo Alto, CA (US)

(21) Appl. No.: 17/995,986

(22) PCT Filed: Apr. 16, 2021
(86) PCT No.: PCT/US2021/027838

§ 371 Date: Oct. 11, 2022

Related U.S. Application Data
(60) Provisional application No. 63/011,742, filed on Apr. 17, 2020.
(60) Provisional application No. 63/150,451, filed on Feb. 17, 2021.

(51) Int. Cl. C07K 14/54; A61K 38/20; A61K 45/06; A61P 35/00

(52) U.S. Cl. CPC C07K 14/5434; A61K 38/208; A61K 38/20; A61K 45/06; A61P 35/00

(57) ABSTRACT

The present disclosure relates generally to compositions and methods for modulating signal transduction mediated by interleukin-12 and interleukin-23. In particular, the disclosure provides novel variants of interleukin-12 subunit p40 with reduced binding affinity to IL-12Rβ1. Also provided are compositions and methods useful for producing such IL-12p40 polypeptide variants, as well as methods for modulating IL-12p40-mediated signaling, and/or for the treatment of conditions associated with perturbations of signal transduction mediated by IL-12p40.

### CROSS-REFERENCE TO RELATED APPLICATIONS

This application claims the benefit of priority to U.S. Provisional Patent Application Ser. No. 63/011,742, filed on Apr. 17, 2020, and U.S. Provisional Patent Application Ser. No. 63/150,451, filed on Feb. 17, 2021. The disclosures of the above-referenced applications are herein expressly incorporated by reference it their entireties, including any drawings.

### STATEMENT REGARDING FEDERALLY SPONSORED R&D

This invention was made with Government support under contracts AI051321 and CA177684 awarded by The National Institutes of Health. The Government has certain rights in the invention.

### INCORPORATION OF THE SEQUENCE LISTING

The material in the accompanying Sequence Listing is hereby incorporated by reference into this application. The accompanying Sequence Listing text file, named 078430-517001WO-Sequence Listing.txt, was created on Apr. 12, 2021 and is 76.5 KB.

### FIELD

The present disclosure relates generally to compositions and methods for modulating signal transduction mediated by IL-12 and IL-23. In particular, the disclosure provides novel IL-12p40 polypeptide variants with reduced binding affinity to IL-12Rβ1. Also provided are compositions and methods useful for producing such IL-12p40 polypeptide variants, as well as methods for modulating IL-12p40-mediated signaling, and/or for the treatment of conditions associated with perturbations of signal transduction mediated by IL-12p40

### BACKGROUND

Biopharmaceuticals or the use of pharmaceutical formulations containing therapeutic protein(s) for the treatment of health conditions and diseases is a core strategy for a number of pharmaceutical and biotechnology companies. For example, several members of the cytokine family have been reported to be effective in the treatment of cancer and play a major role in the development of cancer immunotherapy. Therefore, the cytokine family has been the focus of much clinical work and effort to improve its administration and bio-assimilation.

The IL-12 family cytokines, interleukin-12 (IL-12) and interleukin-23 (IL-23), have become amongst the most promising targets for cancer immunotherapy and autoimmune conditions, respectively. IL-12 and IL-23 complexes share the IL-12p40 cytokine subunit and cell-surface receptor IL-12 receptor beta 1 (IL-12Rβ1) but elicit distinct downstream signaling. In particular, IL-12 signals through a receptor complex of IL-12Rβ1 and IL-12Rβ2 to induce the phosphorylation of STAT4 in both NK cells and activated T cells. STAT4 signaling leads to the expression of interferon-gamma (IFNγ) and enhanced tumor cell killing. In contrast, IL-23 signals through a receptor complex composed of IL-12Rβ1 and IL-23R to promote phosphorylation of STAT3 and expression of IL-17. Although IL-23 plays an important role in immunity against extracellular pathogens, aberrant IL-23 signaling has been associated with the development of multiple autoimmune conditions.

The clinical success of existing therapeutic approaches involving cytokines has been limited due to off-target toxicity and pleiotropy, which is largely due to the fact that cytokines have receptors on both desired and undesired responder cells that counterbalance one another and lead to unwanted side effects. For example, in the case of IL-12, systemic administration of IL-12 leads to toxicity due to NK-cell mediated IFNγ production.

In recent years, cytokine engineering has emerged as a promising strategy to tailor cytokines with desired activities and reduced toxicity. Hence, there is a need for additional approaches to improve properties of IL-12 and IL-23 for their use as a therapeutic agent. In particular, there is a need for variants of IL-12 and IL-23 that can selectively activate certain downstream functions and actions over others, e.g., retain many beneficial properties of IL-12 and IL-23 but lack their known toxic side effects, leading to improved use as anti-cancer agents or immune modulators.

### SUMMARY

The present disclosure relates generally to the field of immunology, including compositions and methods for selectively modulating signal transduction pathway mediated by interleukin-12 (IL-12) and/or interleukin-23 (IL-23). More particularly, in some embodiments, the disclosure provides various recombinant interleukin-12 subunit p40 (IL-12p40) polypeptides with altered binding affinity for its natural receptor, interleukin-12 receptor subunit beta 1 (IL-12Rβ1). As described in greater detail below, IL-12p40 can be modulated to achieve distinct levels of STAT3-mediated signaling and/or STAT4-mediated signaling. Some embodiments of the disclosure provide IL-12p40 partial agonists that can result in a cell-type biased IL-12p40 signaling. Some embodiments provide IL-12p40 partial agonists capable of conferring a cell-type biased IL-12 signaling, for example conferring a reduced IL-12 signaling in natural killer (NK) cells while substantially retaining IL-12 signaling in CD8+ T cells. Also provided are compositions and methods useful for producing such IL-12p40 polypeptide variants, methods for modulating IL-12p40-mediated signaling in a subject, as well as methods for the treatment of conditions associated with perturbations of signal transduction downstream of IL-12p40, such as IL-12 signaling and/or Il-23 signaling.

In one aspect, provided herein are recombinant polypeptides including: (a) an amino acid sequence having one or more 70%, 80%, 90%, 95%, 99%, or 100% sequence identity to an IL-12p40 polypeptide having the amino acid sequence of SEQ ID NO: 1; and further including one or more amino acid substitution at a position corresponding to an amino acid residue selected from the group consisting of X37, X39, X40, X41, X80, X81, X82, X106, X108, X115, X216, X217, X218, and X219 of SEQ ID NO: 1.

Non-limiting exemplary embodiments of the disclosed recombinant polypeptides can include one or more of the following features. In some embodiments, the one or more amino acid substitution is at a position corresponding to an amino acid residue selected from the group consisting of X39, X40, X81, X82, X106, X217, and X219 of SEQ ID NO: 1. In some embodiments, the one or more amino acid substitution is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution, and combinations of any thereof. In some embodiments, the one or more amino acid substitution is at a position corresponding to an amino acid residue selected from the group consisting of W37, P39, D40, A41, K80, E81, F82, K106, E108, D115, H216, K217, L218, and K219 of SEQ ID NO: 1. In some embodiments, the one or more amino acid substitution is at a position corresponding to an amino acid residue selected from the group consisting of W37, P39, D40, E81, F82, K106, K217, and K219 of SEQ ID NO: 1.

In some embodiments, the recombinant polypeptides of the disclosure include an amino acid sequence having at least 70%, 80%, 90%, 95%, 99%, or 100% sequence identity to SEQ ID NO: 1, and further including the amino acid substitutions corresponding to the following amino acid substitutions: (a) W37A; (b) P39A, (c) D40A, (d) E81A (e) F82A, (f) K106A, (g) D109A, (h) K217A, (i) K219A, (j) E81A/F82A, (k) W37A/E81A/F82A, (l) E81A/F82A/K106A, (m) E81A/F82A/K106A/K219A, (n) E81A/F82A/K106A/K217A, (o) 81A/F82A/K106A/E108A/D115A, (p) E81F/F82A, (q) E81K/F82A, (r) E81L/F82A, (s) E81H/F82A, (t) E81S/F82A, (u) E81A/F82A/K106N, (v) E81A/F82A/K106Q, (w) E81A/F82A/K106T, (x) E81A/F82A/K106R or (y) P39A/D40A/E81A/F82A. In some embodiments, the recombinant polypeptides of the disclosure include an amino acid sequence selected from the group consisting of SEQ ID NOS: 3-8 and 13-16.

In one aspect, some embodiments of the disclosure relate to polypeptide including: (a) an amino acid sequence having one or more 70%, 80%, 90%, 95%, 99%, or 100% sequence identity to an IL-12p40 polypeptide having the amino acid sequence of SEQ ID NO: 2; (b) and further including one or more amino acid substitution at a position corresponding to an amino acid residue selected from the group consisting of X37, X39, X40, X41, X80, X81, X82, X106, X108, X115, X216, X217, X218, and X219 of SEQ ID NO: 2. Non-limiting exemplary embodiments of the recombinant polypeptides according to this aspect can include one or more of the following features.

In some embodiments, the one or more amino acid substitution is at a position corresponding to an amino acid residue selected from the group consisting of X37, X39, X40, X81, X82, X106, X217, and X219 of SEQ ID NO: 2. In some embodiments, the one or more amino acid substitution is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution. In some embodiments, the one or more amino acid substitution is at a position corresponding to an amino acid residue selected from the group consisting of W37, P39, D40, A41, K80, E81, F82, K106, E108, D115, H216, K217, L218, and E219 of SEQ ID NO: 2. In some embodiments, the one or more amino acid substitution is at a position corresponding to an amino acid residue selected from the group consisting of W37, P39, D40, E81, F82, K106, K217, and E219 of SEQ ID NO: 2.

In some embodiments, the recombinant polypeptides of the disclosure include an amino acid sequence having one or more 70%, 80%, 90%, 95%, 99%, or 100% sequence identity to SEQ ID NO: 2, and further including the amino acid substitutions corresponding to the following amino acid substitutions: (a) W37A; (b) P39A, (c) D40A, (d) E81A; (e) F82A, (f) K106A, (g) D109A, (h) K217A, (i) E219A, (j) E81A/F82A, (k) W37A/E81A/F82A, (l) E81A/F82A/K106A, (m) E81A/F82A/K106A/K217A, (n) E81F/F82A, (o) E81K/F82A, (p) E81L/F82A, (q) E81H/F82A, (r) E81S/F82A, (s) E81A/F82A/K106N, (t) E81A/F82A/K106Q; (u) E81A/F82A/K106T, (v) E81A/F82A/K106R or (w) P39A/D40A/E81A/F82A. In some embodiments, the recombinant polypeptides include an amino acid sequence selected from the group consisting of SEQ ID NOS: 9-11 and 17-25.

In some embodiments, the recombinant polypeptides of the disclosure have an altered binding affinity for interleukin-12 receptor, beta 1 (IL-12Rβ1) compared to binding affinity of a reference polypeptide lacking the one or more amino acid substitution. In some embodiments, the recombinant polypeptides have a reduced binding affinity for IL-12Rβ1 compared to binding affinity of a reference polypeptide lacking the one or more amino acid substitution. In some embodiments, the recombinant polypeptides have binding affinity for IL-12Rβ1 reduced by about 10% to about 100% compared to binding affinity of a reference polypeptide lacking the one or more amino acid substitution, as determined by surface plasmon resonance (SPR). In some embodiments, the recombinant polypeptides of the disclosure, when combined with an interleukin 12 subunit p35 (IL-12p35) polypeptide, have a reduced capability to stimulate STAT4 signaling compared to a reference polypeptide lacking the one or more amino acid substitution. In some embodiments, the recombinant polypeptides, when combined with an interleukin 23 subunit p19 (IL-23p19) polypeptide, have a reduced capability to stimulate STAT3 signaling compared to a reference polypeptide lacking the one or more amino acid substitution. In some embodiments, the STAT3 signaling and/or STAT4 signaling is determined by an assay selected from the group consisting of by a gene expression assay, a phospho-flow signaling assay, and an enzyme-linked immunosorbent assay (ELISA).

In some embodiments, the one or more amino acid substitution in the disclosed recombinant polypeptides results in a cell-type biased signaling of the downstream signal transduction mediated through interleukin-12 (IL-12) and/or interleukin-23 (IL-23) compared to a reference polypeptide lacking the one or more amino acid substitution. In some embodiments, the cell-type biased signaling includes a reduced capability of the recombinant polypeptide to stimulate IL-12-mediated signaling in NK cells. In some embodiments, the cell-type biased signaling includes a substantially unaltered capability of the recombinant polypeptide to stimulate IL-12 signaling in CD8+ T cells. In some embodiments, the one or more amino acid substitution results in a reduced capability of the recombinant polypeptide to stimulate IL-12 signaling in NK cells while substantially retains its capability to stimulate IL-12 signaling in CD8+ T cells.

In another aspect, provided herein are recombinant nucleic acids, wherein the nucleic acids include a nucleic acid sequence encoding a polypeptide that includes an amino acid sequence having at least 90% sequence identity to the amino acid sequence of the polypeptide of the disclosure.

Non-limiting exemplary embodiments of the disclosed nucleic acid molecules can include one or more of the following features. In some embodiments, the nucleic acid sequence is operably linked to a heterologous nucleic acid sequence. In some embodiments, the nucleic acid molecule is further defined as an expression cassette or an expression vector.

In one aspect, some embodiments of the disclosure relate to recombinant cells, wherein the recombinant cells include one or more of: (a) a recombinant polypeptide of the disclosure; and (b) a recombinant nucleic acid of the disclosure. In some embodiments, the recombinant cell is a eukaryotic cell. In some embodiments, the eukaryotic cell is a mammalian cell. In a related aspect, some embodiments of the disclosure relate to cell cultures including at least one recombinant cell of the disclosure and a culture medium.

In another aspect, some embodiments of the disclosure relate to methods for producing a polypeptide, wherein the methods include: (a) providing one or more recombinant cells of the disclosure; and (b) culturing the one or more recombinant cells in a culture medium such that the cells produce the polypeptide encoded by the recombinant nucleic acid molecule.

In some embodiments, the methods for producing a polypeptide of the disclosure further include isolating and/or purifying the produced polypeptide. In some embodiments, the methods for producing a polypeptide of the disclosure further include structurally modifying the produced polypeptide to increase half-life. In some embodiments, the modification includes one or more alterations selected from the group consisting of fusion to a human Fc antibody fragment, fusion to albumin, and PEGylation. Accordingly, in a related aspect, also provided herein are recombinant polypeptides produced by the method of the disclosure.

In one aspect, some embodiments of the disclosure relate to pharmaceutical compositions, wherein the pharmaceutical compositions include one or more of: (a) a recombinant polypeptide of the disclosure; (b) a recombinant nucleic acid of the disclosure; (c) a recombinant cell of the disclosure; and (d) a pharmaceutically acceptable carrier.

Non-limiting exemplary embodiments of the disclosed pharmaceutical compositions can include one or more of the following features. In some embodiments, the composition includes a recombinant polypeptide of the disclosure and a pharmaceutically acceptable carrier. In some embodiments, the composition includes a recombinant cell of the disclosure and a pharmaceutically acceptable carrier. In some embodiments, the recombinant cell expresses a recombinant polypeptide of the disclosure. Examples of recombinant cells genetically modified to express and secrete therapeutic polypeptides are described previously in, for example, Steidler L. et al., Nature Biotechnology, Vol. 21, No. 7, July 2003 and Oh J. H et al., mSphere, Vol. 5, Issue 3, May/June 2020. In some embodiments, the composition includes a recombinant nucleic acid of the disclosure and a pharmaceutically acceptable carrier. In some embodiments, the composition includes a recombinant cell of the disclosure and a pharmaceutically acceptable carrier.

In one aspect, some embodiments of the disclosure relate to methods for modulating IL-12p40-mediated signaling in a subject, wherein the methods include administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the IL-12p40-mediated signaling includes IL-12-mediated signal transduction. In some embodiments, the IL-12p40-mediated signaling includes IL-23-mediated signal transduction.

Accordingly, some embodiments of the disclosure relate to methods for modulating IL-12-mediated signaling in a subject, wherein the methods include administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the methods further include administering to the subject an IL-12p35 polypeptide, or nucleic acid encoding the IL-12p35 polypeptide.

In some other embodiments, provided herein are methods for modulating IL-23-mediated signaling in a subject, wherein the methods include administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the methods further include administering to the subject an IL-23p19 (p19) polypeptide, or nucleic acid encoding the IL-23p19 polypeptide.

In another aspect, provided herein are various methods for the treatment of a condition in a subject in need thereof, the methods include administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the methods further include administering to the subject a composition including one or more of: (a) an IL-12p35 (p35) polypeptide; (b) an IL-23p19 polypeptide; and (c) nucleic acid encoding (a) or (b).

Non-limiting exemplary embodiments of the disclosed methods for modulating IL-12p40-mediated signaling in a subject and/or for the treatment of a condition in a subject in need thereof can include one or more of the following features. In some embodiments, the recombinant polypeptide has an altered binding affinity for interleukin-12 receptor, subunit beta 1 (IL-12Rβ1) compared to binding affinity of a reference polypeptide lacking the one or more amino acid substitution. In some embodiments, the recombinant polypeptide has a reduced binding affinity for IL-12Rβ1 compared to binding affinity of a reference polypeptide lacking the one or more amino acid substitution. In some embodiments, the recombinant polypeptide has binding affinity for IL-12Rβ1 reduced by about 10% to about 100% compared to binding affinity of a reference polypeptide lacking the one or more amino acid substitution, as determined by surface plasmon resonance (SPR). In some embodiments, the reduced binding affinity of the recombinant polypeptide to IL-12Rβ1 receptor results in a reduction in STAT4-mediated signaling compared to a reference polypeptide lacking the one or more amino acid substitution. In some embodiments, the reduced binding affinity of the recombinant polypeptide to IL-12Rβ1 receptor results in a reduction in STAT3-mediated signaling compared to a reference polypeptide lacking the one or more amino acid substitution. In some embodiments, the STAT3 signaling and/or STAT4 signaling is determined by an assay selected from the group consisting of by a gene expression assay, a phospho-flow signaling assay, and an enzyme-linked immunosorbent assay (ELISA).

In some embodiments, the administered composition results in a cell-type biased signaling of the downstream signal transduction mediated by interleukin-12 (IL-12) and/or by interleukin-23 (IL-23) compared to a reference polypeptide lacking the one or more amino acid substitution. In some embodiments, the cell-type biased signaling includes a reduced capability of the recombinant polypeptide to stimulate IL-12-mediated signaling in NK cells. In some embodiments, the cell-type biased signaling includes a substantially unaltered capability of the recombinant polypeptide to stimulate IL-12 signaling in CD8+ T cells. In some embodiments, the administered composition results in a reduced capability of the recombinant polypeptide to stimulate IL-12 signaling in NK cells while substantially retains its capability to stimulate IL-12 signaling in CD8+ T cells. In some embodiments, the administered composition substantially retains the recombinant polypeptide's capability to stimulate expression of INFγ in CD8+ T cells. In some embodiments, the administered composition enhances antitumor immunity in a tumor microenvironment.

In some embodiments, the subject is a mammal. In some embodiments, the mammal is a human. In some embodiments, the subject has or is suspected of having a condition associated with IL-12p40 mediated signaling. In some embodiments, the IL-12p40 mediated signaling is IL-12 mediated signaling or IL-23 mediated signaling. In some embodiments, the condition is a cancer, an immune disease, or a chronic infection. In some embodiments, the immune disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease.

In some embodiments, provided herein are various methods for the treatment of a condition in a subject in need thereof, wherein the condition is a cancer selected from the group consisting of an acute myeloma leukemia, an anaplastic lymphoma, an astrocytoma, a B-cell cancer, a breast cancer, a colon cancer, an ependymoma, an esophageal cancer, a glioblastoma, a glioma, a leiomyosarcoma, a liposarcoma, a liver cancer, a lung cancer, a mantle cell lymphoma, a melanoma, a neuroblastoma, a non-small cell lung cancer, an oligodendroglioma, an ovarian cancer, a pancreatic cancer, a peripheral T-cell lymphoma, a renal cancer, a sarcoma, a stomach cancer, a carcinoma, a mesothelioma, and a sarcoma.

In some embodiments, the composition is administered to the subject individually as a first therapy or in combination with a second therapy. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

In another aspect, some embodiments of the disclosure relate to kits for the practice of the methods disclosed herein. Some embodiments relate to kits for methods of modulating IL-12p40-mediated signaling in a subject, wherein the kits include one or more of: a recombinant polypeptide of the disclosure; a recombinant nucleic acid of the disclosure; a recombinant cell of the disclosure; and a pharmaceutical composition of the disclosure, and instructions for performing a method as disclosed herein. Some embodiments relate to kits for methods of treating a condition in a subject in need thereof, wherein the kits include one or more of: a recombinant polypeptide of the disclosure; a recombinant nucleic acid of the disclosure; a recombinant cell of the disclosure; and a pharmaceutical composition of the disclosure, and instructions for performing a method as disclosed herein.

Yet another aspect of the disclosure is the use of one or more of: a nucleic acid molecule of the disclosure, a recombinant cell of the disclosure, or a pharmaceutical composition of the disclosure; for treating an individual having or suspected of having a condition associated with a perturbation in IL-12-p40 mediated signal transduction.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E depict the structure of a quaternary IL-23 complex. FIG. 1A: Schematic of IL-12 family cytokine composition and receptor usage. FIG. 1A-1B: Side view of the IL-23 receptor complex FIGS. 1C-1E: Close-up views highlighting the interaction between three interaction sites between IL-23 and receptor subunits.

FIGS. 2A-2H schematically summarize the results from experiments performed to demonstrate that IL-12p40 plays a conserved role in IL-12 and IL-23 signaling. FIG. 2A: IL-12p40 binds to IL-12Rβ1 directly. Surface plasmon resonance (SPR) sensorgrams showing binding of IL-12Rβ1 to immobilized IL-12p40. Dissociation constant (K_{D}) was determined using a steady state affinity model. FIGS. 2B-2C: IL-12 and IL-23 elicit distinct patterns of STAT phosphorylation in CD4+ T cells. CD4+ T cell were activated for 2 days with 2.5 μg αCD3, 5 μg αCD28 and 100 IU/mL rhIL-2, rested overnight and stimulated with IL-12 or IL-23 for 20′ prior to fixation, permeabilization and assessment of STAT phosphorylation by flow cytometry. (D-F) A shared interface in IL-12p40 regulates IL-12 and IL-23 signaling. FIG. 2D: Ribbon diagram showing the interaction between IL-12p40 and IL-12Rβ1. Inset shows amino acid positions targeted for mutagenesis. FIG. 2E: IL-12p40 mutants elicit altered IL-12 pSTAT4 signaling in CD4+ T cells. IL-12p40 variants were coexpressed with IL-12p35 and tested for their ability to stimulate STAT4 signaling in CD4+ T cell blasts. FIG. 2F: IL-12p40 mutants elicit altered IL-23 pSTAT3 signaling in CD4+ T cells. IL-12p40 variants were coexpressed with IL-23p19 and tested for their ability to stimulate STAT3 signaling in CD4+ T cell blasts. FIG. 2G: Ribbon diagram of IL-12p40 with inset showing amino acids at the IL-12Rβ1 interface. FIG. 2H: STAT4 signaling of IL-12p40 variants. IL-12p40 variants were coexpressed with IL-12p35 and tested for their ability to stimulate STAT4 signaling in CD4+ T cell blasts.

FIGS. 3A-3D summarize experiments demonstrating that IL-12p40 regulates STAT4 signaling of murine IL-12. FIG. 3A: Cell-type and activation-state dependent expression of IL-12Rβ1. Flow cytometry plots showing IL-12Rβ1 expression levels measured by mouse IL-12p40 tetramer staining of murine NK cells (CD3−NK1.1+) or CD8+ T cells (CD3+CD8+). Red line indicates 200 nM tetramer staining, gray population represents streptavidin staining alone. Single cell suspension of spleen and lymph nodes from C57/BL6 mice were stained with IL-12p40 tetramer either directly (ex vivo) or following 2-day stimulation with 2.5 μg/mL αCD3 5 μg/mL αCD28 and 100 IU/mL rmIL-2 (blasts). FIG. 3B depicts a sequence alignment of human IL-12p40 polypeptide (SEQ ID NO: 1) and murine IL-12p40 polypeptide (SEQ ID NO: 2). In the alignment, conserved positions are shown with grey shading and positions targeted for mutagenesis are designated with an asterisk. FIGS. 3C-3D: IL-12p40 mutations modulate IL-12 signaling in CD8+ T cell blasts. Dose response (FIG. 3C) and representative histograms at highest concentration (FIG. 3D) of phospho-STAT4 staining following 20′ stimulation with the indicated IL-12 variants (2×Ala: E81A F82A, 3×Ala: E81A F82A K106A, 4×Ala: E81A F82A K106A K217A). Dose-response shows mean and standard error of two biological replicates and is representative of two or more independent experiments.

FIGS. 4A-4C schematically summarize the results from experiments performed to demonstrate that three exemplary IL-12 partial agonists in accordance to some non-limiting embodiments of the disclosure elicit cell-type specific responses based on differential IL-12Rβ1 expression. FIG. 4A: IL-12 partial agonists promote IFNγ production by antigen-specific CD8+ T cells. Representative histograms (left) and quantification (right) of intracellular IFNγ in OT-I CD8+ T cells (CD3+CD8+). OT-I splenocytes were stimulated for 48 hours with 1 μg/mL OVA peptide, 100 IU/mL IL-2 and 1 μM IL-12 variants. In the final four hours, GolgiStop was added to prevent further cytokine secretion. FIG. 4B: IL-12 partial agonists display attenuated IFNγ induction in NK cell. Purified NK cells were stimulated with 50 ng/mL IL-18 with 1 μM IL-12 variants for 48 hours. In the final four hours, GolgiStop was added to prevent further cytokine secretion. FIG. 4C: IL-12 partial agonists display cell-type biased activity. The ratio of αIFNγ AF647 MFI in T cell/NK cells normalized to wild-type IL-12 is shown for IL-12 and partial agonists. Bar graphs show mean and standard error of two biological replicates and are representative of two or more independent experiments. MFI, mean fluorescence intensity.

FIGS. 4D-4G schematically summarize the results from experiments performed to demonstrate that additional exemplary IL-12 partial agonists in accordance to some non-limiting embodiments of the disclosure elicit cell-type specific responses based on differential IL-12Rβ1 expression. FIG. 4D: IL-12p40 mutations modulate IL-12 signaling in CD8+ T cell blasts. Dose response of phospho-STAT4 staining following 20′ stimulation with the indicated IL-12 variants. Dose-response shows mean and standard error of two biological replicates. FIG. 4E: IL-12 partial agonists promote IFNγ production by antigen-specific CD8+ T cells. Quantification of intracellular IFNγ in OT-I CD8+ T cells (CD3+CD8+). OT-I splenocytes were stimulated for 48 hours with 1 μg/mL OVA peptide, 100 IU/mL IL-2 and 1 μM IL-12 variants. In the final four hours, GolgiStop was added to prevent further cytokine secretion. FIG. 4F: IL-12 partial agonists display attenuated IFNγ induction in NK cell. Purified NK cells were stimulated with 50 ng/mL IL-18 with 1 μM IL-12 variants for 48 hours. In the final four hours, GolgiStop was added to prevent further cytokine secretion. FIG. 4G: IL-12 partial agonists display cell-type biased activity. The ratio of αIFNγ AF647 MFI in T cell/NK cells normalized to wild-type IL-12 is shown for IL-12 and partial agonists. Bar graphs show mean and standard error of two biological replicates and are representative of two or more independent experiments. MFI, mean fluorescence intensity.

FIGS. 5A-5C schematically summarize the results from experiments performed to demonstrate that the exemplary IL-12 partial agonists described in FIGS. 4A-4C above promote antigen-specific tumor cell killing. FIGS. 5A-5B: Supernatants from OT-I effectors generated in the presence of IL-12 partial agonists enhance MHC-I upregulation on B16F10 melanoma cells. Dose response (FIG. 5A) and representative histograms (FIG. 5B) of H2-K^{b} surface expression following overnight incubation with OT-I effector supernatants. The arrow indicates the supernatant dilution shown in the representative histograms. OT-I effectors were generated by 72-hour coculture of splenocytes with 1 μg/mL OVA peptide, 100 IU/mL IL-2 and 1 μM IL-12 variants. FIGS. 5C-5D: IL-12 partial agonists enhance potency of antigen-specific tumor cell killing. FIG. 5C: Schematic of the specific killing assay. A 1:1 mixture of wild-type cells and OVA-GFP expressing B16F10 cells were incubated with varying ratios of OT-I effectors and the frequency of OVA-GFP+ cells was used to measure antigen-specific killing. (FIG. 5D) Dose response curves showing specific killing of OT-I effectors generated in the absence of IL-12 or in the presence of the indicated IL-12 variants. Data are represented as mean and standard error of two biological replicates and are representative of two or more independent experiments.

FIGS. 6A-6I schematically summarize the results from experiments performed to characterize mouse IL-12 signaling on NK cells. FIG. 6A: IL-12 partial agonists elicit reduced pSTAT4 signaling in NK cells. MACS purified NK cells were mixed with CellTrace Violet loaded carrier cells and stimulating with IL-12 agonists for 20 minutes. FIG. 6B: IL-18 is required for IL-12 mediated IFNγ induction in NK cells. MACS purified NK cells were stimulated with 50 ng/mL IL-18 and 1 nM IL-12 as indicated. FIG. 6C: IL-12 induces dose dependent IFNγ production in NK cells. NK cells were stimulated as in FIG. 4B with a titration of IL-12 and analyzed for IFNγ induction at 48-hour by intracellular cytokine stain. FIG. 6D: IL-12 agonists elicit dose dependent IFNγ expression in NK cells, related to FIG. 4B. FIG. 6E: 3×Ala and 4×Ala IL-12 partial agonists have reduced secretion of IFNγ by NK cells relative to IL-12. Analysis of IFNγ in supernatant of NK cell cultures by ELISA, related to FIG. 4B. FIGS. 6F-6G: Quantitative PCR (qPCR) of lfng (FIG. 6F) and Tigit (FIG. 6G) from NK cells stimulated with 50 ng/mL IL-18 and 1 μM IL-12 for 8 hours. Ct values were normalized to Gapdh and expressed as fold induction over unstimulated control. Bar graphs show mean±standard deviation of technical triplicates. FIG. 6H: IL-2 pre-activation upregulates IL-12Rβ1 on NK cells. MACS purified NK cells were stimulated with 1000 IU/mL IL-2 for 48 h and stained with 200 nM p40 tetramer (red) or streptavidin control (gray) as in FIG. 3A to identify IL-12Rβ1 expression levels. FIG. 6I: IL-2 enhances IFNγ induction in NK cells but does not synergize with IL-12 partial agonists. MACS purified NK cells were activated with 1000 IU/mL IL-2, 50 ng/mL IL-18 and 1 μM IL-12 agonists for 48 hours. Dashed line indicates IFNγ staining in NK cells stimulated with IL-18 alone. Data are shown as mean±standard deviation of two biological replicates unless otherwise stated and are representative of two or more experiments.

FIGS. 7A-7F schematically summarize the results from experiments performed to characterize various exemplary human IL-12 partial agonists of the disclosure. FIG. 7A: Cell-type and activation-state dependent expression of IL-12Rβ1 in human PBMCs. Flow cytometry plots showing IL-12Rβ1 expression level measured by p40 tetramer staining of human NK cells (CD3−CD56⁺) or CD8⁺ T cells (CD3⁺CD8⁺). Red line indicates 200 nM tetramer staining, gray population represents streptavidin staining alone. For T cell blasts, PBMCs were stimulated for 2 days with 2.5 μg/mL αCD3, 5 μg/mL αCD28, and 100 IU/mL IL-2. FIG. 7B: NK cell and T cell gating scheme. FIGS. 7C-7D: Phospho-flow cytometry of CD8⁺ T cell blasts stimulated with IL-12 partial agonists for 20 minutes. FIG. 7C: Dose-response curves of pSTAT4 signaling in human CD8⁺ T cell blasts. FIG. 7D: Histograms show pSTAT4 staining at 8 nM (IL-12) or 1 μM (2×Ala: E81A/F82A, 3×Ala: E81A/F82A/K106A). FIG. 7E: IL-12 partial agonists support IFNγ secretion by CD8+T cells. MACS isolated CD8+T cells were stimulated with 2 μg/mL αCD3, 0.5 μg/mL αCD28, and 5 ng/mL IL-2 with or without IL-12 agonists. After 48 hours, the supernatant was analyzed for IFNγ ELISA. Dashed line indicates IFNγ level in the absence of IL-12. FIG. 7F: IL-12 partial agonists show attenuated IFNγ production by NK cells. MACS isolated NK cells were stimulated with 100 ng/mL IL-18 with or without IL-12 agonists for 48 hours and the supematant was assayed for IFNγ by ELISA. Conditions in which no IFNγ was detected above background are listed as “n.d.” for not determined. Data are expressed as mean±standard deviation of two biological replicates and are representative of two independent experiments.

FIGS. 7G-7I schematically summarize the results from experiments performed to demonstrate T cell biased of the human IL-12 partial agonist W37A E81A F82A. FIG. 7G: Phospho-flow cytometry of CD8⁺ T cell blasts stimulated with IL-12 partial agonists for 20 minutes. FIG. 7H: IL-12 partial agonists support IFNγ secretion by CD8+T cells. MACS isolated CD8+T cells were stimulated with 2 μg/mL αCD3, 0.5 μg/mL αCD28, and 5 ng/mL IL-2 with or without IL-12 agonists. After 48 hours, the supematant was analyzed for IFNγ ELISA. Dashed line indicates IFNγ level in the absence of IL-12. FIG. 7I: IL-12 partial agonists show attenuated IFNγ production by NK cells. MACS isolated NK cells were stimulated with 100 ng/mL IL-18 with or without IL-12 agonists for 48 hours and the supematant was assayed for IFNγ by ELISA. Data are expressed as mean±standard deviation of two biological replicates and are representative of two independent experiments.

FIGS. 8A-8E schematically summarize the results of experiments performed to validate expression of murine IL-12 agonists from mammalian cells. FIG. 8A: Purification of IL-12 from Expi293F cells. (A) Representative S200 size exclusion chromatography (SEC) of Ni-NTA purified murine IL-12. mAU: milli absorbance units. FIG. 8B: SDS-PAGE of IL-12 following Ni-NTA affinity purification and SEC under reducing (R) and non-reducing (NR) conditions. FIGS. 8C-8F: Characterization of mammalian-expressed mouse IL-12 variants. FIGS. 8C-8D: pSTAT4 staining of CD8+ T cell blasts following a 20 minute stimulation with cytokine. Histograms show pSTAT4 staining at 8 nM for IL-12 and 1 μM for partial agonists. FIG. 8E: Mammalian-expressed IL-12 partial agonists promote IFNγ production by antigen-specific CD8+ T cells. Representative histograms (left) and quantification (right) of intracellular IFNγ in OT-I CD8+ T cells (CD3+CD8+) following 48-hour stimulation with 1 μg/mL OVA peptide (257-264), 0.5 ug/mL αCD28, 100 IU/mL IL-2, and 1 μM IL-12 variants. FIG. 8F: Mammalian-expressed IL-12 partial agonists display attenuated IFNγ induction in NK cell. Purified NK cells were stimulated with 50 ng/mL IL-18 with 1 μM IL-12 variants for 48 hours.

FIGS. 9A-9J schematically summarize the results of experiments performed to illustrate that IL-12 partial agonists elicit cell-type specific responses in vivo. FIG. 9A: Schematic of experimental design. CD8+ T cells from an OT-I TCR transgenic mouse (Thy1.2) were transferred to congenic recipient mice (Thy1.1) on day 0. The following day, mice were immunized subcutaneously with 50 μg OVA (257-264) in Incomplete Freund's Adjuvant (IFA) and daily interperitoneally injection of 30 μg cytokine by was begun. Following 5 days of cytokine treatment, mice were euthanized for analysis of serum IFNγ by ELISA and cell-type profiling in draining lymph nodes by flow cytometry. FIG. 9B: IL-12 but not partial agonists result in weight loss. Mouse weight was monitored daily and normalized to body weight on day 1 prior to initiation of cytokine treatment. FIG. 9C: IL-12 but not partial agonists elevate systemic IFNγ as measured by serum ELISA on day 6. Dashed line represent measurement from unimmunized mice in this and subsequent panels. FIGS. 9D-9E: Immunization increases the frequency of PD-1+ OT-I T cell independent of cytokine treatment. FIG. 9D: Representative FACS plots showing PD-1 expression in OT-I+ T cells identified as CD3+CD8+Thy1.2+. FIG. 9E: Quantification of PD-1+ cells as a frequency of OT-I+ T cells. FIG. 9F: IL-12 but not partial agonists expand OT-I T cells. OT-T cells were identified as Thy1.2+ and expressed as a frequency of total CD8+ T cells. Data were analyzed by Kruskal-Wallis test with Dunn's multiple comparisons. FIG. 9G: IL-12 but not partial agonists increase the frequency of LAG-3+ NK cells. Data were analyzed by Kruskal-Wallis test with Dunn's multiple comparisons. FIG. 9H-J: IL-12 partial agonists preferentially increase the frequency of CD25+ expressing OT-I T cells with reduced activity on NK cells relative to IL-12. FIG. 9H: Representative FACS plots showing CD25 expression in OT-I T cells (top) and NK cells (bottom). FIG. 9I: Quantification of CD25+ OT-I T cells. FIG. 9J Quantification of CD25+ NK cells. Data were analyzed by one-way ANOVA with Tukey's multiple comparisons. Data are expressed as mean±standard deviation of n=5 mice/group and are representative of two independent experiments.

FIGS. 10A-10G schematically summarize the results of experiments performed to illustrate that IL-12 partial agonists support MC-38 anti-tumor response without inducing IL-12 associated toxicity. FIG. 10A: Schematic of experimental design. Mice were implanted with 5×10⁵ MC-38 cells in Matrigel on day 0. Beginning on day 7, mice were administered daily injections of PBS (n=10), 1 μg IL-12 (n=10), 30 μg IL-12 (n=9), 30 μg 2×Ala (n=9), or 30 μg 3×Ala (n=10) as indicated. FIG. 10B: IL-12, but not partial agonists, induces weight loss in tumor-bearing mice. Body weights were normalized to day 7 prior to cytokine treatment. Mice administered 30 μg dose of IL-12 succumbed to cytokine toxicity between days 13 and 15. FIG. 10C: IL-12, but not partial agonists, enhances systemic IFNγ. Serum IFNγ ELISA on day 10, n=5 mice/group. FIG. 10D: IL-12, but not partial agonists, reduce mobility. Cumulative displacement of MC-38 bearing mice following cytokine treatment. Quantitation of 30 second videos captured on day 16. Cumulative displacement was calculated as the sum of ΔX and ΔY over time. Data are shown as mean±standard deviation of n=5 mice/group. FIG. 10E: IL-12 and partial agonists attenuate MC-38 tumor growth. Tumor volumes were compared on day 20 by Kruskal-Wallis test with Dunn's multiple comparisons. FIG. 10F: IL-12 and partial agonists extend survival of MC-38 bearing mice. Kaplan-Meier curves of mice treated with PBS or IL-12 variants. P values from log-rank test were corrected for multiple comparisons using the Holm-Šídák method. FIG. 10G: Individual tumor growth curves of MC-38 bearing mice. Growth curves for PBS-treated mice are shown in gray for comparison with cytokine-treated mice in color. Data are expressed as mean±standard deviation and are representative of two independent experiments.

### DETAILED DESCRIPTION OF THE DISCLOSURE

The present disclosure generally relates to, inter alfa, compositions and methods for selectively modulating signal transduction pathway mediated by interleukin 12 (IL-12) and interleukin 23 (IL-23) in a subject. In particular, the disclosure provides novel IL-12p40 compositions which are based on new insights into how IL-12p40 interacts with its cognate receptor, IL-12Rβ1. As described in greater detail below, IL-12p40-mediated signaling can be modulated by tuning of STAT3-mediated signaling and/or STAT4-mediated signaling. More particularly, in some embodiments, the disclosure provides a new series of IL-12p40 polypeptide variants with modulated binding affinity for interleukin 12 receptor beta 1 subunit (IL-12Rβ1). The disclosure also provides compositions and methods useful for producing such IL-12p40 polypeptides, methods for modulating IL-12p40-mediated signaling in a subject, as well as methods for the treatment of conditions associated with perturbations of signal transduction downstream of the IL-12p40 receptor.

Interleukins IL-12 and IL-23 are heterodimeric cytokines which share the IL-12p40 cytokine subunit and IL-12Rβ1 cell-surface receptor. As described in the Examples below, experiments have been designed and performed to determine the x-ray crystal structure of the complete IL-23 receptor complex, which in turns revealed a modular interaction between IL-12p40 and IL-12Rβ1 that is shared across IL-12 and IL-23. Based on this new structural understanding, several L-12p40 variants with mutation at the interface with IL-12Rβ1 have been generated and tested for their ability to elicit STAT3 and STAT4 signaling. Through this approach, a series of IL-12p40 variants have been identified as being able to produce graded STAT4 signaling in the context of IL-12 and graded STAT3 signaling in the context of IL-23. In the case of IL-12, a number of recombinant IL-12p40 polypeptides described herein were identified to confer a cell-type biased IL-12p40 signaling, for example a reduced capability of the recombinant polypeptides to stimulate IL-12-mediated signaling in NK cells. In some other embodiments, the cell-type based IL-12p40 signaling involves a reduced capability of the recombinant polypeptides to stimulate IL-12 signaling in NK cells while substantially retains its capability to stimulate IL-12 signaling in CD8+ T cells. These new cytokine agonists may have therapeutic utility by preserving the antitumor effects of cytotoxic T cells while reducing the toxicity associated with NK cell activation.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

The term “effective amount”, “therapeutically effective amount”, or “pharmaceutically effective amount” of a subject recombinant polypeptide of the disclosure generally refers to an amount sufficient for a composition to accomplish a stated purpose relative to the absence of the composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “IL-12p40” means wild-type IL-12p40, whether native or recombinant. As such, an IL-12p40 polypeptide refers to any IL-12p40 polypeptide, including but not limited to, a recombinant produced IL-12p40 polypeptide, synthetically produced IL-12p40 polypeptide, IL-12p40 extracted from cells or tissues. An amino acid sequence of wild-type human IL-12p40 precursor is depicted in SEQ ID NO: 1, which is a 328 amino acid residue protein with an N-terminal 22 amino acid signal peptide that can be removed to generate a 306 amino acid mature protein. The amino acid sequence of the mature human IL-12p40 is provided in SEQ ID NO: 26. An amino acid sequence of wild-type murine (Mus musculus) IL-12p40 precursor is depicted in SEQ ID NO: 2, which is a 335 amino acid residue protein with an N-terminal 22 amino acid signal peptide that can be removed to generate 313 amino acid mature protein. The amino acid sequence of the mature murine IL-12p40 is provided in SEQ ID NO: 27. For the purpose of the present disclosure, all amino acid numbering is based on the precursor polypeptide (or pre-protein) sequence of the IL-12p40 protein set forth in SEQ ID NO: 1 (human IL-12p40) or SEQ ID NO: 2 (mouse IL-12p40). However, one of skill in the art would understand that mature proteins are often used to generate recombinant polypeptide constructs.

As used herein, the term “variant” of an IL-12p40 polypeptide refers to a polypeptide in which one or more amino acid substitutions, deletions, and/or insertions are present as compared to the amino acid sequence of a reference IL-12p40 polypeptide, e.g., a wild-type IL-12p40 polypeptide. As such, the term “IL-12p40 polypeptide variant” includes naturally occurring allelic variants or alternative splice variants of an IL-12p40 polypeptide. For example, a polypeptide variant includes the substitution of one or more amino acids in the amino acid sequence of a parent IL-12p40 polypeptide with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.)

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. Thus, a promoter is in operable linkage with a nucleic acid sequence if it can mediate transcription of the nucleic acid sequence. It should be understood that, operably linked elements may be contiguous or non-contiguous. In the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, region, or domains of the recombinant polypeptides of the disclosure may be operably linked to retain proper folding, processing, targeting, expression, binding, and other functional properties of the recombinant polypeptides in the cell. Unless stated otherwise, various modules, domains, and segments of the recombinant polypeptides of the disclosure are operably linked to each other. Operably linked modules, domains, and segments of the recombinant polypeptides of the disclosure may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” or “engineered” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is an IL-12p40 polypeptide variant as disclosed herein.

As used herein, an “individual” or a “subject” includes animals, such as human (e.g., human individuals) and non-human animals. In some embodiments, an “individual” or “subject” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Interleukin-12 Subunit p40 (IL-12p40)

Cytokines are secreted factors that regulate diverse aspects of physiology through multimerization of cell surface receptors and induction of the JAK-STAT signaling pathway. Interleukin-12 (IL-12) and interleukin-23 (IL-23) are heterodimeric cytokines produced by antigen presenting cells in response to pathogen associated molecular patterns and regulate the activation and differentiation of multiple lymphocyte populations. Despite use of the common IL-12p40 subunit and IL-12 receptor beta 1 (IL-12RI31), IL-12 and IL-23 play non-redundant roles in the immune system.

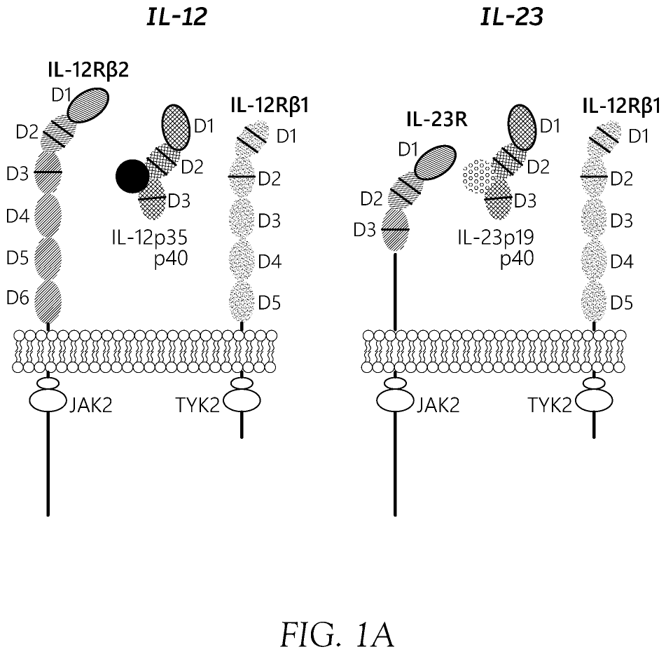

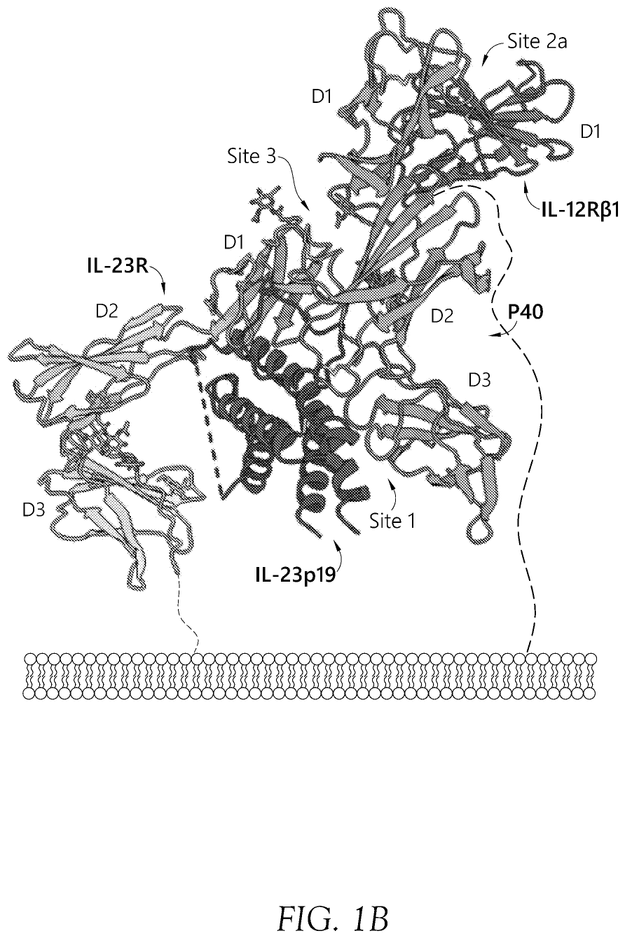

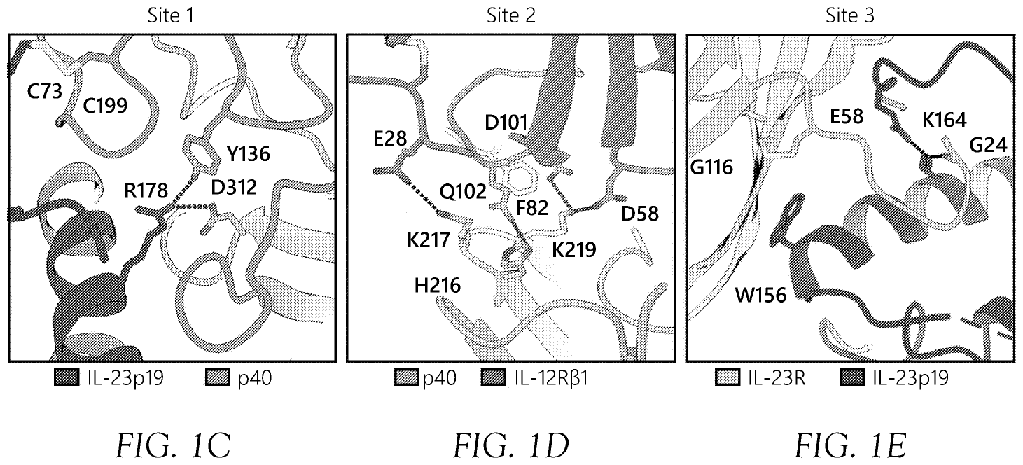

IL-12 signals through a receptor complex of IL-12Rβ1 and IL-12Rβ2 expressed on NK cells and T cells (FIG. 1A). Dimerization of the IL-12 receptor induces activation of receptor associated Janus Kinase (JAK) molecules which phosphorylate each other as well as residues on the intracellular domain of IL-12Rβ2 which serve as docking sites for the SH2 containing signal transducer and activator of transcription 4 (STAT4). Receptor associated STAT4 proteins are then phosphorylated prior to translocating to the nucleus where they promote the expression of IFNγ and the polarization of CD4+ T cells towards a T helper 1 (Th1) phenotype. Given the similarities between immunity to intracellular pathogens and cancer, therapeutic approaches that stimulate Th1 responses, either indirectly through selection of vaccine adjuvants and epitopes, or directly, through administration of IL-12, have been explored in the context of cancer immunotherapy. Despite promise in pre-clinical models, therapeutic efficacy of IL-12 administration has been limited due to toxicity associated with NK cell mediated production of IFNγ.

As schematically shown in FIG. 1A, IL-12 shares its IL-12p40 subunit with IL-23 which signals through a receptor complex formed by IL-12Rβ1 and IL-23 receptor (IL-23R). As a shared receptor for IL-12 and IL-23, IL-12Rβ1 is expressed on T cells, NK cells and monocytes while expression of IL-23R is restricted to γδ T cells and CD4+ T cells. Despite shared use of IL-12Rβ1, IL-12 and IL-23 have distinct phenotypic effects. In CD4+ T cells, IL-23 signaling promotes phosphorylation of STAT3 and stabilization of the IL-17 producing Th17 lineage. While Th17 cells and IL-23 signaling play an important role in the immune response against extracellular pathogens, aberrant Th17 activity has been associated with multiple autoimmune conditions. Indeed, genetic deficiency in either IL-23p19 or IL-12p40 protects mice against experimental autoimmune encephalomyelitis and colitis. Clinically, antagonist antibodies targeting IL-23 have been approved for the treatment of moderate to severe plaque psoriasis, psoriatic arthritis, Crohn's disease and ulcerative colitis, however, many of these antibodies block both IL-12 and IL-23 signaling, leading to complications such as increased risk for infection.

Given the clinical significance of IL-12 and IL-23 signaling, new strategies are needed to specifically modulate this important cytokine axis. However, a lack of structural information about how IL-12 and IL-23 bind to their receptors and initiate downstream signaling has limited the ability to engineer new cytokine variants. To address this, experiments were performed to solve the crystal structure of the complete IL-23 receptor complex (IL-23p19/IL-12p40/IL-23R/IL-12Rβ1) which revealed that IL-12p40 directly engages IL-12Rβ1. As both IL-12Rβ1 and IL-12p40 are shared between IL-12 and IL-23, this interface represents an important feature in complex assembly that initiates signaling of both IL-12 and IL-23. New insights obtained from the crystal structures were then used to design a panel of IL-12 and IL-23 partial agonists which modulate STAT signaling. As demonstrated below, a number of IL-12 agonists have been identified as being capable of preserving CD8+ T cell IFNγ induction and tumor cell killing but elicit reduced IFNγ production from NK cells. Accordingly, by limiting the activity of IL-12 to antigen-specific T cells, IL-12 partial agonists may have therapeutic utility by reducing toxicity associated with NK cell production of IFNγ.

As described in greater detail below, experiments were performed to determine a 3.4 Å resolution crystal structure of the quaternary IL-23 receptor complex which reveals that IL-12p40 engages the shared receptor IL-12Rβ1. This mechanism of receptor assembly is unique for the cytokine superfamily and indicates a shared role for IL-12p40 in IL-12 and IL-23 receptor assembly. Using insights from this newly established structure, additional experiments have been performed to design and test a panel of IL-12 partial agonists which exploit differences in IL-12Rβ1 expression across cell-types to support antigen-specific CD8+ T cell function with reduced activity on NK cells. The present disclosure provides new molecules useful for modulating IL-12p40 mediated signaling, and new approaches for engineering cell-type selective cytokine agonists.

### Compositions of the Disclosure

### A. Recombinant IL-12p40 Polypeptides

As outlined above, some embodiments of the disclosure relate to a new series of IL-12p40 polypeptide variants with altered binding affinity to IL-12Rβ1, and with the properties of partial agonism of the downstream signal transduction mediated through interleukin-12 (IL-12) and/or interleukin-23 (IL-23) in a tissue-specific manner. For example, in some embodiments of the disclosure, the IL-12p40 polypeptide variants disclosed herein confer a reduced capability to stimulate IL-12-mediated signaling in NK cells. In some other embodiments, the IL-12p40 polypeptide variants disclosed herein confer a reduced capability to stimulate IL-12 signaling in NK cells while substantially retains its capability to stimulate IL-12 signaling in CD8+ T cells.

In one aspect, some embodiments of the disclosure relate to recombinant polypeptides that include: (a) an amino acid sequence having at least 70% sequence identity to an IL-12p40 polypeptide having the amino acid sequence of SEQ ID NO: 1, and further including (b) one or more amino acid substitutions in the sequence of SEQ ID NO: 1.

Non-limiting exemplary embodiments of the recombinant polypeptides disclosed herein can include one or more of the following features. In some embodiments, the recombinant polypeptides include an amino acid sequence having at least 75%, at least 80%, at least 85%, at least 90%, at least 95%, at least 96%, at least 97%, at least 98%, or at least 99% to the sequence of SEQ ID NO: 1. In some embodiments, the recombinant polypeptides include an amino acid sequence having 100% sequence identify to the sequence of SEQ ID NO: 1.

In some embodiments, the amino acid sequence of the recombinant polypeptides disclosed herein further include one or more amino acid substitutions at a position corresponding to an amino acid residue selected from the group consisting of X37, X39, X40, X41, X80, X81, X82, X106, X108, X115, X216, X217, X218, and X219 of SEQ ID NO: 1. In some embodiments, the amino acid sequence of the recombinant polypeptides further include about 1 to about 14 amino acid substitutions at a position corresponding to an amino acid residue selected from the group consisting of X37, X39, X40, X41, X80, X81, X82, X106, X108, X115, X216, X217, X218, and X219 of SEQ ID NO: 1. In some embodiments, the amino acid sequence of the recombinant polypeptides further include about 1 to about 5, about 2 to about 8, about 3 to about 10, about 4 to about 12, about 5 to about 15, about 3 to about 5, about 7 to about 5, or about 3 to about 12 amino acid substitutions at a position corresponding to an amino acid residue selected from the group consisting of X37, X39, X40, X41, X80, X81, X82, X106, X108, X115, X216, X217, X218, and X219 of SEQ ID NO: 1. In some embodiments, the amino acid sequence of the recombinant polypeptides further include at least 1, at least 2, at least 3, at least 4, at least 5, at least 6, at least 7, at least 8, at least 9, at least 10, at least 11, at least 12, at least 13, at least 14, or at least 15 amino acid substitutions at a position corresponding to an amino acid residue selected from the group consisting of X37, X39, X40, X41, X80, X81, X82, X106, X108, X115, X216, X217, X218, and X219 of SEQ ID NO: 1.

In some embodiments, the amino acid sequence of the recombinant polypeptides disclosed herein further include one or more amino acid substitutions at a position corresponding to an amino acid residue selected from the group consisting of X37, X39, X40, X81, X82, X106, X217, and X219 of SEQ ID NO: 1. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of X37, X39, X40, X81, X82, X106, X217, and X219 of SEQ ID NO: 1. Exemplary IL-12p40 polypeptide variants of the disclosure can include substitutions of 1, 2, 3, 4, 5, 6, 7, 8, 9, 10 or more amino acids in the sequence of SEQ ID NO: 1. In some embodiments, the amino acid sequence of the recombinant polypeptides further include 1, 2, 3, 4, or 5 amino acid substitutions at a position corresponding to an amino acid residue selected from the group consisting of X37, X39, X40, X81, X82, X106, X217, and X219 of SEQ ID NO: 1. In some embodiments, the amino acid sequence of the recombinant polypeptides disclosed herein further include one or more amino acid substitutions at a position corresponding to an amino acid residue selected from the group consisting of X81, X82, X106, X217, and X219 of SEQ ID NO: 1. In some embodiments, the amino acid sequence of the recombinant polypeptides disclosed herein further include includes a combination of amino acid substitutions at positions corresponding to amino acid residues X39, X40, X81, X82 of SEQ ID NO: 1.

In accordance with this aspect and other aspects of the disclosure, any such amino acid substitutions in an IL-12p40 polypeptide result in an IL-12p40 variant that has an altered binding affinity for IL-12Rβ1 compared to binding affinity of the parent IL-12p40 polypeptide lacking such substitutions. For example, the IL-12p40 polypeptide variants disclosed herein can have increased affinity or decreased affinity for IL-12Rβ1 or can have an affinity for IL-12Rβ1 which is identical or similar to that of wild-type IL-12p40. The IL-12p40 polypeptide variants disclosed herein can also include conservative modifications and substitutions at other positions of IL-12p40 (e.g., those that have a minimal effect on the secondary or tertiary structure of the IL-12p40 variants). Such conservative substitutions include those described by Dayhoff in The Atlas of Protein Sequence and Structure 5 (1978), and by Argos in EMBO J, 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the amino acid substitution(s) in the amino acid sequence of the recombinant IL-12p40 polypeptides disclosed herein is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution, and combinations of any thereof. Non-limiting examples of the amino acid substitutions in the recombinant IL-12p40 polypeptides disclosed herein are provided in Tables 1 below.

TABLE 1
Exemplary amino acid substitutions in the recombinant
IL-12p40 polypeptides of the disclosure.

| Position of | Original | Exemplary |
|---|---|---|
| SEQ ID NO: 1 | amino acid | substitute amino acid |
| 37 | W | A, D, K, V, I, L, M, G, S, T |
| 39 | P | A, V, I, L, M, G, S, T |
| 40 | D | A, V, I, L, M, G, S, T, R, H, K |
| 80 | K | A, V, I, L, M, G, S, T, D, E |
| 81 | E | A, V, I, L, M, G, S, T, R, H, K |
| 82 | F | A, V, I, L, M, G, S, T |
| 106 | K | A, V, I, L, M, G, S, T, D, E |
| 108 | E | A, V, I, L, M, G, S, T, R, H, K |
| 109 | D | A, V, I, L, M, G, S, T, R, H, K |
| 115 | D | A, V, I, L, M, G, S, T, R, H, K |
| 216 | H | A, V, I, L, M, G, S, T, D, E |
| 217 | K | A, V, I, L, M, G, S, T, D, E |
| 218 | L | A, V, I, M, G, S, T, D, E |
| 219 | K | A, V, I, L, M, G, S, T, D, E |

In some embodiments, the recombinant polypeptides include an amino acid sequence having at least 70% sequence identity to the sequence of SEQ ID NO: 1, and further include an amino acid substitution corresponding an amino acid residue selected from the group consisting of W37, P39, D40, A41, K80, E81, F82, K106, E108, D115, H216, K217, L218, and K219 of SEQ ID NO: 1. In some embodiments, the recombinant polypeptides include an amino acid sequence having at least 75%, at least 80%, at least 85%, at least 90%, at least 95%, at least 96%, at least 97%, at least 98%, at least 99%, or at least 100% sequence identity to the sequence of SEQ ID NO: 1, and further include an amino acid substitution corresponding an amino acid residue selected from the group consisting of W37, P39, D40, A41, K80, E81, F82, K106, E108, D115, H216, K217, L218, and K219 of SEQ ID NO: 1. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of W37, P39, D40, A41, K80, E81, F82, K106, E108, D115, H216, K217, L218, and K219 of SEQ ID NO: 1.

In some embodiments, the amino acid substitution(s) is at a position corresponding to an amino acid residue selected from the group consisting of W37, P39, D40, A41, K80, E81, F82, K106, E108, D115, H216, K217, L218, and K219 of SEQ ID NO: 1. In some embodiments, the amino acid substitution(s) is at a position corresponding to an amino acid residue selected from the group consisting of W37, P39, D40, E81, F82, K106, K217, and K219 of SEQ ID NO: 1. In some embodiments, the amino acid sequence of the recombinant polypeptides disclosed herein further include one or more amino acid substitutions at a position corresponding to an amino acid residue selected from the group consisting of E81, F82, K106, K217, and K219 of SEQ ID NO: 1. In some embodiments, the amino acid sequence of the recombinant polypeptides disclosed herein further include includes a combination of amino acid substitutions at positions corresponding to amino acid residues W37, P39, D40, E81, F82 of SEQ ID NO: 1. In some embodiments, the amino acid sequence includes an amino acid substitution corresponding to amino acid residue E81, F82, K106, K217, and K219 of SEQ ID NO: 1. In some embodiments, the polypeptides of the disclosure include an amino acid sequence having at least 70% sequence identity to SEQ ID NO: 1, and further include the amino acid substitutions corresponding to the following amino acid substitutions: (a) W37A; (b) P39A, (c) D40A, (d) E81A (e) F82A, (f) K106A, (g) D109A, (h) K217A, (i) K219A, (j) E81A/F82A, (k) W37A/E81A/F82A, (l) E81A/F82A/K106A, (m) E81A/F82A/K106A/K219A, (n) E81A/F82A/K106A/K217A, (o) 81A/F82A/K106A/E108A/D115A, (p) E81F/F82A, (q) E81K/F82A, (r) E81L/F82A, (s) E81H/F82A, (t) E81S/F82A, (u) E81A/F82A/K106N, (v) E81A/F82A/K106Q, (w) E81A/F82A/K106T, (x) E81A/F82A/K106R or (y) P39A/D40A/E81A/F82A. In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 1, and further include the amino acid substitutions corresponding to the following amino acid substitutions: (a) W37A; (b) P39A, (c) D40A, (d) E81A (e) F82A, (f) K106A, (g) D109A, (h) K217A, (i) K219A, (j) E81A/F82A, (k) W37A/E81A/F82A, (l) E81A/F82A/K106A, (m) E81A/F82A/K106A/K219A, (n) E81A/F82A/K106A/K217A, (o) 81A/F82A/K106A/E108A/D115A, (p) E81F/F82A, (q) E81K/F82A, (r) E81L/F82A, (s) E81H/F82A, (t) E81S/F82A, (u) E81A/F82A/K106N, (v) E81A/F82A/K106Q, (w) E81A/F82A/K106T, (x) E81A/F82A/K106R or (y) P39A/D40A/E81A/F82A. In some embodiments, the polypeptides of the disclosure include an amino acid sequence having 100% sequence identity to SEQ ID NO: 1, and further include the amino acid substitutions corresponding to the following amino acid substitutions: (a) W37A; (b) P39A, (c) D40A, (d) E81A (e) F82A, (f) K106A, (g) D109A, (h) K217A, (i) K219A, (j) E81A/F82A, (k) W37A/E81A/F82A, (l) E81A/F82A/K106A, (m) E81A/F82A/K106A/K219A, (n) E81A/F82A/K106A/K217A, (o) 81A/F82A/K106A/E108A/D115A, (p) E81F/F82A, (q) E81K/F82A, (r) E81L/F82A, (s) E81H/F82A, (t) E81S/F82A, (u) E81A/F82A/K106N, (v) E81A/F82A/K106Q, (w) E81A/F82A/K106T, (x) E81A/F82A/K106R or (y) P39A/D40A/E81A/F82A. In some embodiments, the recombinant polypeptides of the disclosure include an amino acid sequence having at least 70%, 80%, 90%, 95%, 99%, or 100% sequence identity to an IL-12p40 polypeptide having the amino acid sequence selected from the group consisting of SEQ ID NOS: 3-8 and 13-16.

In another aspect, some embodiments of the disclosure relate to recombinant polypeptides that include: (a) an amino acid sequence having at least 70% sequence identity to an IL-12p40 polypeptide having the amino acid sequence of SEQ ID NO: 2, and further including (b) one or more amino acid substitutions in the sequence of SEQ ID NO: 2. Non-limiting exemplary embodiments of the recombinant polypeptides according to this aspect can include one or more of the following features. In some embodiments, the recombinant polypeptides include an amino acid sequence having at least 75%, at least 80%, at least 85%, at least 90%, at least 95%, at least 96%, at least 97%, at least 98%, or at least 99% to the sequence of SEQ ID NO: 2. In some embodiments, the recombinant polypeptides include an amino acid sequence having 100% sequence identify to the sequence of SEQ ID NO: 2.

In some embodiments, the amino acid sequence of the recombinant polypeptides further include one or more amino acid substitutions at a position corresponding to an amino acid residue selected from the group consisting of X37, X39, X40, X41, X80, X81, X82, X106, X108, X115, X216, X217, X218, and X219 of SEQ ID NO: 2. In some embodiments, the amino acid sequence of the recombinant polypeptides further include about 1 to about 14 amino acid substitutions at a position corresponding to an amino acid residue selected from the group consisting of X37, X39, X40, X41, X80, X81, X82, X106, X108, X115, X216, X217, X218, and X219 of SEQ ID NO: 2. In some embodiments, the amino acid sequence of the recombinant polypeptides further include about 1 to about 5, about 2 to about 8, about 3 to about 10, about 4 to about 12, about 5 to about 15, about 3 to about 5, about 7 to about 5, or about 3 to about 12 amino acid substitutions at a position corresponding to an amino acid residue selected from the group consisting of X37, X39, X40, X41, X80, X81, X82, X106, X108, X115, X216, X217, X218, and X219 of SEQ ID NO: 2. In some embodiments, the amino acid sequence of the recombinant polypeptides further include at least 1, at least 2, at least 3, at least 4, at least 5, at least 6, at least 7, at least 8, at least 9, at least 10, at least 11, at least 12, at least 13, at least 14, or at least 15 amino acid substitutions at a position corresponding to an amino acid residue selected from the group consisting of X37, X39, X40, X41, X80, X81, X82, X106, X108, X115, X216, X217, X218, and X219 of SEQ ID NO: 2.

In some embodiments, the amino acid sequence of the recombinant polypeptides disclosed herein further include one or more amino acid substitutions at a position corresponding to an amino acid residue selected from the group consisting of X39, X40, X81, X82, X106, X217, and X219 of SEQ ID NO: 2. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of X39, X40, X81, X82, X106, X217, and X219 of SEQ ID NO: 2. Exemplary IL-12p40 polypeptide variants of the disclosure can include substitutions of 1, 2, 3, 4, 5, 6, 7, 8, 9, 10 or more amino acids in the sequence of SEQ ID NO: 2. In some embodiments, the amino acid sequence of the recombinant polypeptides further include 1, 2, 3, 4, or 5 amino acid substitutions at a position corresponding to an amino acid residue selected from the group consisting of X39, X40, X81, X82, X106, X217, and X219 of SEQ ID NO: 2. In some embodiments, the amino acid substitution(s) is at a position corresponding to an amino acid residue selected from the group consisting of X81, X82, X106, and X217 of SEQ ID NO: 2. In some embodiments, the amino acid sequence includes a combination of amino acid substitutions at positions corresponding to amino acid residues X81, X82, and X106 of SEQ ID NO: 2. In some embodiments, the amino acid sequence includes a combination of amino acid substitutions at positions corresponding to amino acid residues X81, X82, X106, and X217 of SEQ ID NO: 2.

In accordance with this aspect and other aspects of the disclosure, any such amino acid substitution(s) in an IL-12p40 polypeptide result in an IL-12p40 variant that has an altered binding affinity for IL-12Rβ1 compared to binding affinity of the parent IL-12p40 polypeptide lacking such substitution(s). For example, the IL-12p40 polypeptide variants disclosed herein can have increased affinity or decreased affinity for IL-12Rβ1 or can have an affinity for IL-12Rβ1 which is identical or similar to that of wild-type IL-12p40. The IL-12p40 polypeptide variants disclosed herein can also include conservative modifications and substitutions at other positions of IL-12p40 (e.g., those that have a minimal effect on the secondary or tertiary structure of the IL-12p40 variants). Such conservative substitutions include those described by Dayhoff 1978, supra, and by Argos 1989, supra. For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the amino acid substitution(s) in the amino acid sequence of the recombinant IL-12p40 polypeptides disclosed herein is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution, and combinations of any thereof. In some embodiments, the amino acid substitutions(s) in the amino acid sequence of the recombinant IL-12p40 polypeptides disclosed herein includes an alanine substitution. Non-limiting examples of the amino acid substitutions in the recombinant IL-12p40 polypeptides disclosed herein are provided in Tables 2 below.

TABLE 2
Exemplary amino acid substitutions in the recombinant
IL-12p40 polypeptides of the disclosure.

| Position of | Original | Exemplary |
|---|---|---|
| SEQ ID NO: 2 | amino acid | substitute amino acid |
| 37 | W | A, D, K, V, I, L, M, G, S, T |
| 39 | P | A, V, I, L, M, G, S, T |
| 40 | D | A, V, I, L, M, G, S, T, R, H, K |
| 80 | K | A, V, I, L, M, G, S, T, D, E |
| 81 | E | A, V, I, L, M, G, S, T, R, H, K |
| 82 | F | A, V, I, L, M, G, S, T |
| 106 | K | A, V, I, L, M, G, S, T, D, E |
| 108 | E | A, V, I, L, M, G, S, T, R, H, K |
| 109 | N | A, V, I, L, M, G, S, T, R, H, K |
| 115 | E | A, V, I, L, M, G, S, T, R, H, K |
| 215 | Q | A, V, I, L, M, G, S, T, D, E |
| 216 | N | A, V, I, L, M, G, S, T, D, E |
| 217 | K | A, V, I, L, M, G, S, T, D, E |

In some embodiments, the recombinant polypeptides include an amino acid sequence having at least 70% sequence identity to the sequence of SEQ ID NO: 2, and further include an amino acid substitution corresponding an amino acid residue selected from the group consisting of W37, P39, D40, A41, K80, E81, F82, K106, E108, D115, H216, K217, L218, and E219 of SEQ ID NO: 2. In some embodiments, the recombinant polypeptides include an amino acid sequence having at least 75%, at least 80%, at least 85%, at least 90%, at least 95%, at least 96%, at least 97%, at least 98%, at least 99%, or at least 100% sequence identity to the sequence of SEQ ID NO: 2, and further include an amino acid substitution corresponding an amino acid residue selected from the group consisting of W37, P39, D40, A41, K80, E81, F82, K106, E108, D115, H216, K217, L218, and E219 of SEQ ID NO: 2. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of W37, P39, D40, A41, K80, E81, F82, K106, E108, D115, H216, K217, L218, and E219 of SEQ ID NO: 2.

In some embodiments, the amino acid substitution(s) is at a position corresponding to an amino acid residue selected from the group consisting of W37, P39, D40, A41, K80, E81, F82, K106, E108, D115, H216, K217, L218, and E219 of SEQ ID NO: 2. In some embodiments, the amino acid substitution(s) is at a position corresponding to an amino acid residue selected from the group consisting of W37, P39, D40, E81, F82, K106, K217, and E219 of SEQ ID NO: 2. In some embodiments, the amino acid substitution(s) is at a position corresponding to an amino acid residue selected from the group consisting of E81, F82, K106, and K217 of SEQ ID NO: 2. In some embodiments, the amino acid sequence includes a combination of amino acid substitutions at positions corresponding to amino acid residues E81, F82, and K106 of SEQ ID NO: 2. In some embodiments, the amino acid sequence includes a combination of amino acid substitutions at positions corresponding to amino acid residues E81, F82, K106, and K217 of SEQ ID NO: 2. In some embodiments, the polypeptides of the disclosure include an amino acid sequence having at least 70% sequence identity to SEQ ID NO: 2, and further include the amino acid substitutions corresponding to the following amino acid substitutions: (a) W37A; (b) P39A, (c) D40A, (d) E81A; (e) F82A, (f) K106A, (g) D109A, (h) K217A, (i) E219A, (j) E81A/F82A, (k) W37A/E81A/F82A, (l) E81A/F82A/K106A, (m) E81A/F82A/K106A/K217A, (n) E81F/F82A, (o) E81K/F82A, (p) E81L/F82A, (q) E81H/F82A, (r) E81S/F82A, (s) E81A/F82A/K106N, (t) E81A/F82A/K106Q; (u) E81A/F82A/K106T, (v) E81A/F82A/K106R or (w) P39A/D40A/E81A/F82A.

In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 2, and further include the amino acid substitutions corresponding to the following amino acid substitutions: (a) W37A; (b) P39A, (c) D40A, (d) E81A; (e) F82A, (f) K106A, (g) D109A, (h) K217A, (i) E219A, (j) E81A/F82A, (k) W37A/E81A/F82A, (l) E81A/F82A/K106A, (m) E81A/F82A/K106A/K217A, (n) E81F/F82A, (o) E81K/F82A, (p) E81L/F82A, (q) E81H/F82A, (r) E81S/F82A, (s) E81A/F82A/K106N, (t) E81A/F82A/K106Q; (u) E81A/F82A/K106T, (v) E81A/F82A/K106R or (w) P39A/D40A/E81A/F82A. In some embodiments, the polypeptides of the disclosure include an amino acid sequence having 100% sequence identity to SEQ ID NO: 2, and further include the amino acid substitutions corresponding to the following amino acid substitutions: (a) W37A; (b) P39A, (c) D40A, (d) E81A; (e) F82A, (f) K106A, (g) D109A, (h) K217A, (i) E219A, (j) E81A/F82A, (k) W37A/E81A/F82A, (l) E81A/F82A/K106A, (m) E81A/F82A/K106A/K217A, (n) E81F/F82A, (o) E81K/F82A, (p) E81L/F82A, (q) E81H/F82A, (r) E81S/F82A, (s) E81A/F82A/K106N, (t) E81A/F82A/K106Q; (u) E81A/F82A/K106T, (v) E81A/F82A/K106R or (w) P39A/D40A/E81A/F82A. In some embodiments, the recombinant polypeptides of the disclosure include an amino acid sequence having at least 70%, 80%, 90%, 95%, 99%, or 100% sequence identity to an IL-12p40 polypeptide having the amino acid sequence selected from the group consisting of SEQ ID NOS: 9-11 and 17-25.

In some embodiments, the amino acid substitution(s) in the sequence of the recombinant IL-12p40 polypeptide disclosed herein results in an altered affinity of the recombinant IL-12p40 polypeptide for IL-12Rβ1 and modulates IL-12p40 binding for IL-12Rβ1. The term “modulating”, in relation to the binding activity of an IL-12p40 polypeptide refers to a change in the binding affinity of the polypeptide for IL-12Rβ1. Modulation includes both increase (e.g., induce, stimulate) and decrease (e.g., reduce, inhibit), or otherwise affecting the binding affinity of the polypeptide. In some embodiments, the amino acid substitution(s) increases IL-12Rβ1-binding affinity of the recombinant IL-12p40 polypeptide compared to a reference IL-12p40 polypeptide lacking the amino acid substitution(s). In some embodiments, the amino acid substitution(s) in the sequence of the recombinant IL-12p40 polypeptide disclosed herein reduces IL-12Rβ1-binding affinity of the recombinant IL-12p40 polypeptide compared to a reference IL-12p40 polypeptide lacking the amino acid substitution(s).

The binding activity of recombinant polypeptides of the disclosure, including the IL-12p40 polypeptide variants described herein, can be assayed by any suitable method known in the art. For example, the binding activity of an IL-12p40 polypeptide variant disclosed herein and its cognate ligands (e.g., IL-12Rβ1, IL-p35, and IL-23p19) can be determined by Scatchard analysis (Munsen et al. Analyt. Biochem. 107:220-239, 1980). Specific binding may also be assessed using techniques known in the art including but not limited to competition ELISA, Biacore® assays and/or KinExA® assays. A polypeptide that preferentially binds or specifically binds to a target ligand is a concept well understood in the art, and methods to determine such specific or preferential binding are also known in the art.

A variety of assay formats may be used to select a recombinant IL-12p40 polypeptide that binds a ligand of interest (e.g., IL-12Rβ1, IL-p35, and/or IL-23p19). For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify a polypeptide that specifically reacts with a receptor or a ligand binding portion thereof, that specifically binds with a cognate ligand or binding partner. Generally, a specific or selective binding reaction will be at least twice the background signal or noise, more typically more than 10 times background, more than 20 times background, even more typically, more than 50 times background, more than 75 times background, more than 100 times background, yet more typically, more than 500 times background, even more typically, more than 1000 times background, and even more typically, more than 10,000 times background.

One of ordinary skill in the art will appreciate that binding affinity can also be used as a measure of the strength of a non-covalent interaction between two binding partners, e.g., an IL-12p40 polypeptide and an IL-12Rβ1 polypeptide. In some instance, binding affinity is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=kd/kₐ. The value of the dissociation constant can be determined directly by well-known methods and can be computed even for complex mixtures by methods such as those set forth in Caceci et al. (Byte 9: 340-362, 1984). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of the IL-12p40 polypeptide variants of the present disclosure towards target receptors are known in the art, including for example, ELISAs, Western blots, RIAs, and flow cytometry analysis, and other assays exemplified in the Examples. The binding kinetics and binding affinity of the IL-12p40 polypeptide variants also can be assessed by standard assays known in the art, such as Surface Plasmon Resonance (SPR), e.g. by using a Biacore™ system, or KinExA. In some embodiments, the binding affinity of the IL-12p40 polypeptide variant of the disclosure to IL-12Rβ1, IL-p35, and/or IL-23p19 is determined by a solid-phase receptor binding assay (Matrosovich M N et al., Methods Mol Biol. 865:71-94, 2012). In some embodiments, the binding affinity of the IL-12p40 polypeptide variant of the disclosure to IL-12Rβ1, IL-p35, and/or IL-23p19 is determined by a Surface Plasmon Resonance (SPR) assay.

In some embodiments, the amino acid substitution(s) in the sequence of the recombinant IL-12p40 polypeptides disclosed herein reduces IL-12Rβ1-binding affinity of the recombinant IL-12p40 polypeptides by about 10% to about 100% compared to a reference IL-12p40 polypeptide lacking the amino acid substitution(s). In some embodiments, the recombinant IL-12p40 polypeptides have binding affinity for IL-12Rβ1 reduced by at least about 10%, about 20%, about 30%, about 40%, about 50%, about 60%, about 70%, about 80%, about 90%, or by at least about 95% compared to a reference IL-12p40 polypeptide lacking the amino acid substitution(s). In some embodiments, the recombinant IL-12p40 polypeptides have binding affinity for IL-12Rβ1 reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to IL-12Rβ1-binding affinity of a reference IL-12p40 polypeptide lacking the amino acid substitution(s). In some embodiments, the binding affinity of the IL-12p40 polypeptide variant of the disclosure to IL-12Rβ1, IL-p35, and/or IL-23p19 is determined by a Surface Plasmon Resonance (SPR) assay.

In some embodiments, the recombinant IL-12p40 polypeptide variants disclosed herein, when combined with an IL-12p35 polypeptide, have a reduced capability to stimulate STAT4 signaling compared to a reference IL-12p40 polypeptide lacking the amino acid substitution(s). In some embodiments, the capability of the recombinant IL-12p40 polypeptide variants to stimulate STAT4 signaling is reduced by at least about 10%, about 20%, about 30%, about 40%, about 50%, about 60%, about 70%, about 80%, about 90%, or by at least about 95% compared to a reference IL-12p40 polypeptide lacking the amino acid substitution(s). In some embodiments, the capability of the recombinant IL-12p40 polypeptide variants to stimulate STAT4 signaling is reduced by about 10% to about 100% compared to a reference IL-12p40 polypeptide lacking the amino acid substitution(s). In some embodiments, the capability of the recombinant IL-12p40 polypeptide variants to stimulate STAT4 signaling is reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12p40 polypeptide lacking the amino acid substitution(s).

In some embodiments, the recombinant IL-12p40 polypeptide variants disclosed herein, when combined with an IL-23p19 polypeptide, have a reduced capability to stimulate STAT3 signaling compared to a reference IL-12p40 polypeptide lacking the amino acid substitution(s). In some embodiments, the capability of the recombinant IL-12p40 polypeptide variants to stimulate STAT3 signaling is reduced by at least about 10%, about 20%, about 30%, about 40%, about 50%, about 60%, about 70%, about 80%, about 90%, or by at least about 95% compared to a reference IL-12p40 polypeptide lacking the amino acid substitution(s). In some embodiments, the capability of the recombinant IL-12p40 polypeptide variants to stimulate STAT3 signaling is reduced by about 10% to about 100% compared to a reference IL-12p40 polypeptide lacking the amino acid substitution(s). In some embodiments, the capability of the recombinant IL-12p40 polypeptide variants to stimulate STAT4 signaling is reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12p40 polypeptide lacking the amino acid substitution(s).

In principle, there are no particular restrictions in regard to the assays and methodologies that can be used to determine STAT3 signaling and/or STAT4 signaling. Exemplary methodologies suitable for the compositions and methods disclosed herein include, but are not limited to, phospho-flow signaling assays, an enzyme-linked immunosorbent assays (ELISA), and any techniques known in the art for assaying expression of downstream genes. In some embodiments, the modulation in STAT3 signaling and/or STAT4 signaling can be determined by a phospho-flow signaling assay, such as phospho-flow cytometry assay described in Examples 4 and 5.

In some embodiments, the recombinant IL-12p40 polypeptide variants disclosed herein confer a cell-type biased signaling of the downstream signal transduction mediated through IL-12p40 compared to a referenced IL-12p40 polypeptide lacking the amino acid substitution(s). In some embodiments, the recombinant IL-12p40 polypeptide variants disclosed herein confers a cell-type biased signaling of the downstream signal transduction mediated through IL-12. In some embodiments, the recombinant IL-12p40 polypeptide variants disclosed herein confers a cell-type biased signaling of the downstream signal transduction mediated through IL-23. In some embodiments, the recombinant IL-12p40 polypeptide variants disclosed herein confers a cell-type biased signaling of the downstream signal transduction mediated through IL-12 and IL-23.

In the case of IL-12, as described in greater detail below, certain partial agonistic IL-12p40 variants of the disclosure demonstrate selectivity for T cells versus NK cells and therefore are predicted to be less toxic than natural IL-12, which is in clinical development for cancer by many companies and the limitation is its toxicity. Without being bound to any particular theory, it is contemplated that these IL-12 partial agonists will have therapeutic utility in cancer immunotherapy by uncoupling toxicity associated with cytokine pleiotropy. As shown in the Example 4 below, certain IL-12 partial agonists disclosed herein demonstrate reduced affinity for IL-12Rβ1 which retain activity on antigen-specific CD8+ T cells but show reduced (e.g., impaired) stimulation of NK cells. As NK cell mediated IFNγ is thought to be responsible for IL-12 toxicity, these new agonists are predicted to preserve anti-tumor effects of IL-12 stimulation with reduced toxicity. In the case of IL-23, it is contemplated that the partial agonistic IL-12p40 variants of the disclosure will have therapeutic utility in the treatment of autoimmune disease by allowing graded control of IL-23 signaling.

Complementary to current therapeutic approaches which rely on antibody blockade of IL-12p40 which inhibits IL-12 and IL-23 signaling, the partial agonist IL-12p40 variants of the disclosure demonstrates that by modulating the affinity of IL-23 for IL-12Rβ1, IL-23 partial agonists can be used to specifically regulate IL-23 signaling without impacting IL-12.

Accordingly, some embodiments of the disclosure provide recombinant IL-12p40 polypeptides that confer a cell-type biased signaling of the downstream signal transduction mediated through IL-12 compared to a referenced IL-12 polypeptide lacking the amino acid substitution(s), wherein the cell-type biased signaling includes a reduced capability of the recombinant polypeptide to stimulate IL-12-mediated signaling in NK cells. In some embodiments, the capability of the recombinant IL-12p40 polypeptide variants disclosed herein to stimulate IL-12-mediated signaling in NK cells is reduced by at least about 10%, about 20%, about 30%, about 40%, about 50%, about 60%, about 70%, about 80%, about 90%, or by at least about 95% compared to a reference IL-12p40 polypeptide lacking the amino acid substitution(s). In some embodiments, the cell-type biased signaling includes a substantially unaltered capability of the recombinant polypeptide to stimulate IL-12 signaling in CD8+ T cells. In some embodiments, the capability of the recombinant IL-12p40 polypeptide variants disclose herein to stimulate IL-12-mediated signaling in CD8+ T cells is unaltered, e.g., the same or substantially the same compared a reference IL-12p40 polypeptide lacking the amino acid substitution(s). In some embodiments, the recombinant IL-12p40 polypeptide variants disclose herein confer a reduced capability of the recombinant polypeptide to stimulate IL-12 signaling in NK cells while substantially retains its capability to stimulate IL-12 signaling in CD8+ T cells, and promote antigen-specific killing od target cells, as described in Example 5 below.

### B. Nucleic Acids

In one aspect, provided herein are various nucleic acid molecules including nucleotide sequences encoding the recombinant IL-12p40 polypeptides the disclosure, including expression cassettes, and expression vectors containing these nucleic acid molecules operably linked to heterologous nucleic acid sequences such as, for example, regulator sequences which allow in vivo expression of the recombinant IL-12p40 polypeptide in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acid molecules of the disclosure include a nucleotide sequence encoding a polypeptide which includes an amino acid sequence having at least 90%, at least 95%, at least 96%, at least 97, at least 98%, at least 99%, or at least 100% sequence identity to the amino acid sequence of a recombinant polypeptide as disclosed herein. In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-12p40 polypeptide having the amino acid sequence of SEQ ID NO: 1; and further including (b) one or more amino acid substitution at a position corresponding to an amino acid residue selected from the group consisting of X37, X39, X40, X41, X80, X81, X82, X106, X108, X115, X216, X217, X218, and X219 of SEQ ID NO: 1. In some embodiments, the amino acid substitution(s) is at a position corresponding to an amino acid residue selected from the group consisting of X39, X40, X81, X82, X106, X217, and X219 of SEQ ID NO: 1. In some embodiments, the amino acid sequence includes an amino acid substitution corresponding to amino acid residue X81, X82, X106, X217, and X219 of SEQ ID NO: 1. In some embodiments, the amino acid sequence includes a combination of amino acid substitutions at positions corresponding to amino acid residues X39, X40, X81, X82 of SEQ ID NO: 1. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 1, and further include an amino acid substitution corresponding an amino acid residue selected from the group consisting of W37, P39, D40, A41, K80, E81, F82, K106, E108, D115, H216, K217, L218, and K219 of SEQ ID NO: 1. In some embodiments, the amino acid substitution(s) is at a position corresponding to an amino acid residue selected from the group consisting of W37, P39, D40, E81, F82, K106, K217, and K219 of SEQ ID NO: 1. In some embodiments, the amino acid sequence includes an amino acid substitution corresponding to amino acid residue E81, F82, K106, K217, and K219 of SEQ ID NO: 1. In some embodiments, the amino acid sequence includes a combination of amino acid substitutions at positions corresponding to amino acid residues P39, D40, E81, F82 of SEQ ID NO: 1. In some embodiments, the nucleic acid molecules of the disclosure include a nucleotide sequence encoding a polypeptide that includes an amino acid sequence having at least 70% sequence identity to SEQ ID NO: 1, and further include the amino acid substitutions corresponding to the following amino acid substitutions: (a) W37A; (b) P39A, (c) D40A, (d) E81A (e) F82A, (f) K106A, (g) D109A, (h) K217A, (i) K219A, (j) E81A/F82A, (k) W37A/E81A/F82A, (l) E81A/F82A/K106A, (m) E81A/F82A/K106A/K219A, (n) E81A/F82A/K106A/K217A, (o) 81A/F82A/K106A/E108A/D115A, (p) E81F/F82A, (q) E81K/F82A, (r) E81L/F82A, (s) E81H/F82A, (t) E81S/F82A, (u) E81A/F82A/K106N, (v) E81A/F82A/K106Q, (w) E81A/F82A/K106T, (x) E81A/F82A/K106R or (y) P39A/D40A/E81A/F82A. In some embodiments, the nucleic acid molecules of the disclosure include a nucleotide sequence encoding a polypeptide that includes an amino acid sequence having at least 70%, 80%, 90%, 95%, 99%, or 100% sequence identity to an IL-12p40 polypeptide having the amino acid sequence selected from the group consisting of SEQ ID NOS: 3-8 and 13-16.

In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-12p40 polypeptide having the amino acid sequence of SEQ ID NO: 2; and further including (b) one or more amino acid substitution at a position corresponding to an amino acid residue selected from the group consisting of X37, X39, X40, X41, X80, X81, X82, X106, X108, X115, X216, X217, X218, and X219 of SEQ ID NO: 2. In some embodiments, the polypeptide further includes an additional amino acid substitution at a position corresponding to an amino acid residue selected from the group consisting of X39, X40, X81, X82, X106, X217, and X219 of SEQ ID NO: 2. In some embodiments, the amino acid substitution(s) is at a position corresponding to an amino acid residue selected from the group consisting of X81, X82, X106, and X217 of SEQ ID NO: 2. In some embodiments, the amino acid sequence includes a combination of amino acid substitutions at positions corresponding to amino acid residues X81, X82, and X106 of SEQ ID NO: 2. In some embodiments, the amino acid sequence includes a combination of amino acid substitutions at positions corresponding to amino acid residues X81, X82, X106, and X217 of SEQ ID NO: 2. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 2, and further include an amino acid substitution corresponding an amino acid residue selected from the group consisting of W37, P39, D40, A41, K80, E81, F82, K106, E108, D115, H216, K217, L218, and E219 of SEQ ID NO: 2. In some embodiments, the amino acid sequence includes an additional amino acid substitution at a position corresponding to an amino acid residue selected from the group consisting of W37, P39, D40, E81, F82, K106, K217, and E219 of SEQ ID NO: 2. In some embodiments, the amino acid substitution(s) is at a position corresponding to an amino acid residue selected from the group consisting of E81, F82, K106, and K217 of SEQ ID NO: 2. In some embodiments, the amino acid sequence includes a combination of amino acid substitutions at positions corresponding to amino acid residues E81, F82, and K106 of SEQ ID NO: 2. In some embodiments, the amino acid sequence includes a combination of amino acid substitutions at positions corresponding to amino acid residues E81, F82, K106, and K217 of SEQ ID NO: 2.

In some embodiments, the nucleic acid molecules of the disclosure include a nucleotide sequence encoding a polypeptide that includes an amino acid sequence having at least 70% sequence identity to SEQ ID NO: 2, and further include the amino acid substitutions corresponding to the following amino acid substitutions: (a) W37A; (b) P39A, (c) D40A, (d) E81A; (e) F82A, (f) K106A, (g) D109A, (h) K217A, (i) E219A, (j) E81A/F82A, (k) W37A/E81A/F82A, (l) E81A/F82A/K106A, (m) E81A/F82A/K106A/K217A, (n) E81F/F82A, (o) E81K/F82A, (p) E81L/F82A, (q) E81H/F82A, (r) E81S/F82A, (s) E81A/F82A/K106N, (t) E81A/F82A/K106Q; (u) E81A/F82A/K106T, (v) E81A/F82A/K106R or (w) P39A/D40A/E81A/F82A. In some embodiments, the nucleic acid molecules of the disclosure include a nucleotide sequence encoding a polypeptide that includes an amino acid sequence having at least 70%, 80%, 90%, 95%, 99%, or 100% sequence identity to an IL-12p40 polypeptide having the amino acid sequence selected from the group consisting of SEQ ID NOS: 9-11 and 17-25.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the recombinant polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a bacculorival vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acid molecules encoding any recombinant polypeptide or IL-12p40 polypeptide variant disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan.

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, baculoviral vectors, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoamidite-based synthesis), or combinations or modifications of the nucleotides within these types of nucleic acids. In addition, the nucleic acid molecules can be double-stranded or single-stranded (e.g., either a sense or an anti sense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Recombinant Cells and Cell Cultures

The recombinant nucleic acids of the present disclosure can be introduced into a cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the recombinant cell's genome, or can be episomally replicating, or present in the recombinant cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the recombinant cell is an immune system cell, e.g., a lymphocyte (e.g., a T cell or NK cell), or a dendritic cell. In some embodiments, the immune cell is a B cell, a monocyte, a NK cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte. In some embodiments, the cell can be obtained by leukapheresis performed on a sample obtained from a subject. In some embodiments, the subject is a human subject. In some embodiments, the human subject is a patient.

Non-limiting examples of suitable cell lines include Trichoplusia ni cells, Spodotera frupperda insect cells, Expi293F cells, N-acetylglucosaminyltransferase I (GnTI) deficient HEK293S cells, HEK-293T (ATCC #CRL-3216), HT-29 (ATCC #HTB-38), Panc-1 (ATCC #CRL-1469), HepG2 (ATCC #HB-8065), B16F10 melanoma cells (ATCC #CRL-6475), and EC4 cells.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### D. Methods for Producing an IL-12p40 Polypeptide

In another aspect, some embodiments of the disclosure relate to various methods for producing a recombinant polypeptide of the disclosure, the methods include: (a) providing one or more recombinant cells of the disclosure; and culturing the recombinant cell(s) in a culture medium such that the cells produce the polypeptide encoded by the recombinant nucleic acid molecule. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods further include isolating and/or purifying the produced polypeptide. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

In some embodiments, the modification includes one or more alterations selected from the group consisting of fusion to a human Fc antibody fragment, fusion to albumin, and PEGylation. For example, any of the recombinant polypeptides disclosed herein can be prepared as fusion or chimeric polypeptides that include a recombinant polypeptide and a heterologous polypeptide (e.g., a polypeptide that is not IL-12p40 or a variant thereof). Exemplary heterologous polypeptides can increase the circulating half-life of the chimeric polypeptide in vivo, and may, therefore, further enhance the properties of the recombinant polypeptides of the disclosure. In various embodiments, the heterologous polypeptide that increases the circulating half-life may be a serum albumin, such as human serum albumin, or the Fc region of the IgG subclass of antibodies that lacks the IgG heavy chain variable region. Exemplary Fc regions can include a mutation that inhibits complement fixation and Fc receptor binding, or it may be lytic, e.g., able to bind complement or to lyse cells via another mechanism, such as antibody-dependent complement lysis (ADCC).

In some embodiments, the “Fc region” can be a naturally occurring or synthetic polypeptide that is homologous to the IgG C-terminal domain produced by digestion of IgG with papain. IgG Fc has a molecular weight of approximately 50 kDa. The recombinant fusion polypeptides of the disclosure can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases. In some embodiments, the recombinant fusion protein (e.g., an IL-12p40 partial agonist or antagonist as described herein) includes an IgG1, IgG2, IgG3, or IgG4 Fc region.

The Fc region can be “lytic” or “non-lytic”, but is typically non-lytic. A non-lytic Fc region typically lacks a high affinity Fc receptor binding site and a C′1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fe receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In other embodiments, the recombinant fusion polypeptide can include a recombinant IL-12p40 polypeptide of the disclosure and a polypeptide that functions as an antigenic tag, such as a FLAG sequence. FLAG sequences are recognized by biotinylated, highly specific, anti-FLAG antibodies. In some embodiments, the recombinant fusion polypeptide further includes a C-terminal c-myc epitope tag.

In other embodiments, the recombinant fusion polypeptide includes a recombinant IL-12p40 polypeptide of the disclosure and a heterologous polypeptide that functions to enhance expression or direct cellular localization of the IL-12p40 polypeptide, such as the Aga2p agglutinin subunit.

In other embodiments, a fusion polypeptide including a recombinant IL-12p40 polypeptide of the disclosure and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are known in the art.

In some embodiments, the recombinant IL-12p40 polypeptides of the disclosure can be modified with one or more polyethylene glycol (PEG) molecules to increase its half-life. The term “PEG” as used herein means a polyethylene glycol molecule. In its typical form, PEG is a linear polymer with terminal hydroxyl groups and has the formula HO—CH₂CH₂—(CH₂CH₂O)n-CH₂CH₂—OH, where n is from about 8 to about 4000.

Generally, “n” is not a discrete value but constitutes a range with approximately Gaussian distribution around an average value. The terminal hydrogen may be substituted with a capping group such as an alkyl or alkanol group. PEG can have at least one hydroxy group, more preferably it is a terminal hydroxy group. This hydroxy group is can be attached to a linker moiety which can react with the peptide to form a covalent linkage. Numerous derivatives of PEG exist in the art. The PEG molecule covalently attached to the recombinant IL-12p40 polypeptides of the present disclosure may be approximately 10,000, 20,000, 30,000, or 40,000 daltons average molecular weight. PEGylation reagents may be linear or branched molecules and may be present singularly or in tandem. The PEGylated IL-12p40 polypeptides of the present disclosure can have tandem PEG molecules attached to the C-terminus and/or the N-terminus of the peptide. The term “PEGylation” as used herein means the covalent attachment of one or more PEG molecules, as described above, to a molecule such as the IL-12p40 polypeptides of the present disclosure. In some embodiments, the recombinant polypeptides of the disclosure, e.g., IL-12p40 (p40) variant polypeptides may be PEGylated at one or more of positions corresponding to W37, P39, D40, K80, K106, E108, D115, H216, and K217 of SEQ ID NO: 1 or SEQ ID NO: 2.

### E. Pharmaceutical Compositions

The recombinant polypeptides, nucleic acids, recombinant cells, and/or cell cultures of the disclosure can be incorporated into compositions, including pharmaceutical compositions. Such compositions generally include one or more of the recombinant polypeptides, nucleic acids, recombinant cells, and/or cell cultures as provided and described herein, and a pharmaceutically acceptable excipient, e.g., carrier. In some embodiments, the pharmaceutical compositions of the disclosure are formulated for the treating, preventing, ameliorating a disease such as cancer, or for reducing or delaying the onset of the disease.

Accordingly, one aspect of the present disclosure relates to pharmaceutical compositions that include one or more of the following: (a) a recombinant polypeptide of the disclosure; (b) a recombinant nucleic acid of the disclosure; (c) a recombinant cell of the disclosure; and (d) a pharmaceutically acceptable carrier. In some embodiments, the pharmaceutical compositions include (a) a recombinant polypeptide of the disclosure and (b) a pharmaceutically acceptable carrier. In some embodiments, the pharmaceutical compositions include (a) a recombinant cell of the disclosure and (b) a pharmaceutically acceptable carrier. In some embodiments, the pharmaceutical compositions include (a) a recombinant nucleic acid of the disclosure and (b) a pharmaceutically acceptable carrier. In some embodiments, the recombinant nucleic acid is encapsulated in a viral capsid or a lipid nanoparticle.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the subject recombinant polypeptides of the disclosure are prepared with carriers that will protect the recombinant polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions (including liposomes targeted to infected cells with monoclonal antibodies to viral antigens) can also be used as pharmaceutically acceptable carriers. These can be prepared according to methods known to those skilled in the art.

As described in greater detail below, the recombinant polypeptides of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the recombinant polypeptides of the disclosure can be fused to a stable protein, such as, albumin. For example, human albumin is known as one of the most effective proteins for enhancing the stability of polypeptides fused thereto and there are many such fusion proteins reported.

Acylation

In some embodiments, one or both of the components of the dimeric IL-12 or IL-23 polypeptides comprising a IL-12p40 polypeptide variant polypeptide of the present disclosure may be acylated by conjugation to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine —SH groups such as DHHC proteins catalyze S-palmitoylation. Palmitoleylation of serine and threonine residues is typically achieved enzymatically using PORCN enzymes.

Acetylation

In some embodiments, the IL-12 or IL-23 comprising a IL-12p40 variant polypeptide of the present disclosure are acetylated at either or both N-termini of the dimeric IL-12 or IL-23 molecule by enzymatic reaction with N-terminal acetyltransferase and, for example, acetyl CoA. Alternatively, or in addition to N-terminal acetylation, a subunit of the IL-12(p35/p40) variant or IL-23(p19/p40) variant polypeptides of the present disclosure is acetylated at one or more lysine residues, e.g. by enzymatic reaction with a lysine acetyltransferase. See, for example Choudhary et al. (2009) Science 325 (5942):834L2 ortho840.

Fc Fusion

In some embodiments, when the dimeric IL-12(p35/p40) variant or IL-23(p19/p40) variant polypeptide may be provided in the format of an Fc fusion wherein each component of the dimeric molecule is provided on individual subunits of a dimeric Fc molecule. In some embodiments, the IL-12p40 fusion protein may incorporate an Fc region derived from the IgG subclass of antibodies that lacks the IgG heavy chain variable region. The “Fc region” can be a naturally occurring or synthetic polypeptide that is homologous to the IgG C-terminal domain produced by digestion of IgG with papain. IgG Fc has a molecular weight of approximately 50 kDa. The mutant conjugate polypeptides may include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases. In certain embodiments, the Fc fusion protein (e.g., an IL-12p35 or IL-23p19 and IL-12p40 variant) includes an IgG1, IgG2, IgG3, or IgG4 Fc region. Exemplary Fc regions can include a mutation that inhibits complement fixation and Fc receptor binding, or it may be lytic, i.e., able to bind complement or to lyse cells via another mechanism such as antibody-dependent complement lysis (ADCC).

In some embodiments, the IL-12p35 or IL-23p19 and p40 variant fusion protein comprises a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The IL-12p40 variants may include the entire Fc region, or a smaller portion that retains the ability to extend the circulating half-life of a chimeric polypeptide of which it is a part. In addition, full-length or fragmented Fc regions can be variants of the wild type molecule. In a typical implementation, each monomer of the dimeric Fc carries a component of the dimeric IL-12(p35/p40) variant or IL-23(p19/p40) variant polypeptide.

Knob/Hole Fc Conjugates

In some embodiments, when the dimeric IL-12(p35/p40) variant or IL-23(p19/p40) variant polypeptide may be provided in the format of an Fc fusion wherein each component of the dimeric molecule is provided on individual subunits of a dimeric Fc molecule wherein the dimeric Fc molecule subunits are engineered to possess a “knob-into-hole modification” such that each subunit of the IL-12 (i.e., p35 and p40 variant) or IL-23 (p19 and p40 variant) are expressed as a fusion protein (optionally comprising an intervening linker sequence between the p35 or p19 sequence and the Fc subunit sequence) is expressed on a “knob” or “hole” Fc subunit and the p40 variant polypeptide is expressed on the complementary “knob” or “hole” Fc subunit. The knob-into-hole modification is more fully described in Ridgway, et al. (1996) Protein Engineering 9(7):617-621 and U.S. Pat. No. 5,731,168. Generally, the knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g. tyrosine or tryptophan) creating a projection from the surface (“knob”) and ii) in the CH3 domain of a second heavy chain, an amino acid residue is replaced with an amino acid residue having a smaller side chain (e.g. alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an p40 variant of the present disclosure) on a first Fc monomer with a “knob” modification and a second polypeptide (p19 or p35) on the second Fc monomer possessing a “hole” modification, or vice versa, to facilitate the expression and surface presentation of heterodimeric IL-12(p35/p40) variant or IL-23(p19/p40) variant polypeptide Fc fusion constructs.

Pegylation

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the average molecular weight of said PEG, or PEG derivative, is about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the average molecular weight of said PEG, or PEG derivative, is about 5 kD, about 10 kD, about 20 kD, about 30 kD, about 40 kD, about 50 kD, about 60 kD, about 70 kD, or about 80 kD. In some embodiments, the average molecular weight of said PEG, or PEG derivative, is about 40 kD. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-succinimidyl glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the recombinant polypeptides of the disclosure. In some embodiments, the pegylation reagent is polyethylene glycol with an average molecular weight of about 5 kD, about 10 kD, about 20 kD, about 30 kD, about 40 kD, about 50 kD, about 60 kD, about 70 kD, or about 80 kD covalently bound to the N-terminal methionine residue of the recombinant polypeptides of the disclosure. In some embodiments, the pegylation reagent is polyethylene glycol with an average molecular weight of about 40 kD covalently bound to the N-terminal methionine residue of the recombinant polypeptides of the disclosure.

Accordingly, in some embodiments, the recombinant polypeptides of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the disclosed recombinant polypeptide. In some embodiments, the PEGylated or PASylated polypeptide contains a PEG or PAS moiety on only one amino acid. In other embodiments, the PEGylated or PASylated polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated polypeptide may be coupled directly to PEG or PAS (e.g., without a linking group) through an amino group, a sulfhydryl group, a hydroxyl group, or a carboxyl group. In some embodiments, the recombinant polypeptide of the disclosure is covalently bound to a polyethylene glycol with an average molecular weight ranging from about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 1 150 kD to about 200 kD. In some embodiments, the recombinant polypeptide of the disclosure is covalently bound to a polyethylene glycol with an average molecular weight of about 5 kD, about 10 kD, about 20 kD, about 30 kD, about 40 kD, about 50 kD, about 60 kD, about 70 kD, or about 80 kD. In some embodiments, the recombinant polypeptide of the disclosure is covalently bound to a polyethylene glycol with an average molecular weight of about 40 kD.

Incorporation of Site-Specific PEGylation Sites

In some embodiments, the recombinant polypeptides of the disclosure, e.g., IL-12p40 (p40) variant polypeptides may be modified by the incorporation of non-natural amino acids with non-naturally occurring amino acid side chains to facilitate site specific conjugation (e.g., PEGylation) as described in, for example, U.S. Pat. Nos. 7,045,337; 7,915,025; Dieters, et al. (2004) Bioorganic and Medicinal Chemistry Letters 14(23):5743-5745; Best, M (2009) Biochemistry 48(28): 6571-6584. In some embodiments, cysteine residues may be incorporated at various positions within the recombinant polypeptides of the disclosure to facilitate site-specific PEGylation via the cysteine side chain as described in, for example, Dozier and Distefano (2015) International Journal of Molecular Science 16(10): 25831-25864.

In certain embodiments, the present disclosure provides IL-12p40 variant polypeptides comprising incorporation of one or more amino acids enabling site specific PEGylation (e.g., cysteine or non-natural amino acid) of the present disclosure, wherein the amino acid substitution for site specific PEGylation site is not in the interface between the p40/p35 (IL-12) or p40/p19 (IL-23) interface.

In some embodiments the incorporation of the site-specific amino acid modification are incorporated at IL-12p40 amino acid positions other than amino acid residues W37, P39, D40, A41, K80, E81, F82, K106, E108, D115, H216, K217, L218, and K219 of SEQ ID NO: 1 (i.e., residues W15, P17, D18, A19, K58, E59, F60, K84, E86, D93, H194, K195, L196, and K197 when numbered in accordance with the mature IL-12p40 protein lacking the signal peptide). In some embodiments, the present disclosure provides compositions comprising human p40 variants comprising site-specific amino acid substitutions to enable site specific conjugation (e.g. PEGylation) are at amino acid positions W37, P39, D40, A41, K80, E81, F82, K106, E108, D115, H216, K217, L218, and K219 numbered in accordance with SEQ ID NO: 1.

In some embodiments the incorporation of the site-specific amino acid modification are incorporated at IL-12p40 amino acid positions other than amino acid residues W37, P39, D40, A41, K80, E81, F82, K106, E108, D115, H216, K217, L218, and E219 of SEQ ID NO: 2 (i.e., residues W15, P17, D18, A19, K58, E59, F60, K84, E86, D93, H194, K195, L196, and E197 when numbered in accordance with the mature IL-12p40 protein lacking the signal peptide). In some embodiments, the present disclosure provides compositions comprising human p40 variants comprising site-specific amino acid substitutions to enable site specific conjugation (e.g. PEGylation) are at amino acid positions W37, P39, D40, A41, K80, E81, F82, K106, E108, D115, H216, K217, L218, and K219 numbered in accordance with SEQ ID NO: 2.

IL-12 and IL-23 Partial Agonists via Site Specific PEGylation at Interface

In some embodiments, the interaction of the IL-12p40 with the p35 or p19 proteins may be modulated by incorporation of site specific pegylation at the amino acid locations described herein at the IL-12p40 interface. The incorporation of non-natural amino acids (or cysteine residues) that facilitate site specific PEGylation at one or more of positions corresponding to residues W37, P39, D40, A41, K80, E81, F82, K106, E108, D115, H216, K217, L218, and K219 of SEQ ID NO: 1 or SEQ ID NO: 2 (i.e., residues W15, P17, D18, A19, K58, E59, F60, K84, E86, D93, H194, K195, L196, and K197 when numbered in accordance with the mature IL-12p40 protein lacking the signal peptide, i.e., the sequence of SEQ ID NO: 26 or SEQ ID NO: 27) provide IL-12p40 variant polypeptides with modulated binding to the p19 and/or p35 subunits resulting in IL-12(p35/p40) variant or IL-23(p19/p40) variant molecules having partial agonist activity. In such instances where PEG molecules are incorporated at the interface, so as to not completely disrupt the binding of the IL-12p40 variant with the p19 or p35 proteins thereby ablating activity, the PEG is typically a low molecular weight PEG species of from about 1 kDa, alternatively about 2 kDa, alternatively about 3 kDa, alternatively about 4 kDa, alternatively about 5 kDa, alternatively about 6 kDa, alternatively about 7 kDa, alternatively about 8 kDa, alternatively about 9 kDa, alternatively about 10 kDa, alternatively about 12 kDa, alternatively about 15 kDa, or alternatively about 20 kD.

### Methods of the Disclosure

Administration of any one of the therapeutic compositions described herein, e.g., recombinant polypeptides (e.g., IL-12p40 polypeptide variants), nucleic acids, recombinant cells, and pharmaceutical compositions, can be used to treat subjects in the treatment of relevant diseases, such as cancers, immune diseases, and chronic infections. In some embodiments, recombinant polypeptides, IL-12p40 polypeptide variants, nucleic acids, recombinant cells, and pharmaceutical compositions as described herein can be incorporated into therapeutic agents for use in methods of treating an individual who has, who is suspected of having, or who may be at high risk for developing one or more autoimmune disease or conditions associated with perturbations in IL-12p40 signaling. Exemplary autoimmune disease or conditions can include, without limitation, cancers, immune diseases, and chronic infection. In some embodiments, the individual is a patient under the care of a physician.

Accordingly, in one aspect, some embodiments of the disclosure relate to methods for modulating IL-12p40-mediated signaling in a subject, wherein the methods include administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the composition includes a therapeutically effective amount of the recombinant IL-12p40 polypeptide of the disclosure. In some embodiments, the composition includes a therapeutically effective amount of the recombinant nucleic acid of the disclosure. As described above, IL-12p40 is a shared subunit of interleukin-12 and interleukin-23. Accordingly, in some embodiments, provided herein are methods for modulating signal transduction mediated by IL-12 in a subject. In some embodiments, the methods of modulating IL-12 signaling as disclosed herein further include administering to the subject an IL-12p35 polypeptide of an IL-12 complex. In some embodiments, the methods further include administering to the subject nucleic acid molecules encoding the IL-12p35 subunit of the IL-12 complex. In some embodiments, the nucleic acids encoding the IL-12p35 polypeptide are encoded by different nucleic acid molecules (e.g., vectors). In some embodiments, the IL-12p40 polypeptide and the IL-12p35 polypeptide are encoded by nucleic acid sequences that are operably linked to one another within a single expression cassette (e.g., polycistronic expression cassette).

In some other embodiments, the disclosure provides methods for modulating signal transduction mediated by IL-23 in a subject. In some embodiments, the methods of modulating IL-23 signaling as disclosed herein further include administering to the subject an IL-23p19 subunit of an IL-23 complex. In some embodiments, the methods further include administering to the subject nucleic acids encoding the IL-12p35 polypeptide of the IL-12 complex. In some embodiments, the nucleic acid molecules encoding the IL-12p35 polypeptide are encoded by different nucleic acid molecules (e.g., vectors). In some embodiments, the IL-12p40 polypeptide and the IL-23p19 polypeptide are encoded by nucleic acid sequences that are operably linked to one another within a single expression cassette (e.g., polycistronic expression cassette).

In another aspect, some embodiments of the disclosure relate to methods for the treatment of a condition in a subject in need thereof, wherein the methods includes administering to the subject a composition including one or more of: (a) a recombinant IL-12p40 polypeptide of the disclosure; (b) a recombinant nucleic acid of the disclosure; (c) a recombinant cell of the disclosure; and (d) a pharmaceutically composition of the disclosure. In some embodiments, the composition includes a therapeutically effective amount of the recombinant IL-12p40 polypeptide of the disclosure. In some embodiments, the composition includes a therapeutically effective amount of the recombinant nucleic acid of the disclosure. In some embodiments, the treatment methods disclosed herein further include administration of an IL-12p35 subunit of an IL-12 complex. In some embodiments, the treatment methods disclosed herein further include administration of an IL-23p19 subunit of an IL-23 complex. In some embodiments, the treatment methods disclosed herein further include administering to the subject nucleic acid molecules encoding an IL-12p35 subunit of an IL-12 complex and/or nucleic acid molecules encoding an IL-23p19 subunit of an IL-23 complex.

In some embodiments, the disclosed pharmaceutical composition is formulated to be compatible with its intended route of administration. The recombinant polypeptides of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant polypeptides of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutically effective amount of a subject recombinant polypeptide of the disclosure (e.g., an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU). The compositions can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject recombinant polypeptides of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

Non-limiting exemplary embodiments of the disclosed methods for modulating IL-12p40-mediated signaling in a subject and/or for the treatment of a condition in a subject in need thereof can include one or more of the following features.

In some embodiments, the administered composition results in an altered binding affinity of the recombinant IL-12p40 polypeptide for IL-12Rβ1 compared to binding affinity of a reference polypeptide lacking the amino acid substitution(s). In some embodiments, the administered composition results in a reduced binding affinity of the recombinant IL-12p40 polypeptide for IL-12Rβ1 compared to binding affinity of a reference polypeptide lacking the amino acid substitution(s). In some embodiments, the recombinant IL-12p40 polypeptide has binding affinity for IL-12Rβ1 reduced by about 10% to about 100% compared to binding affinity of a reference polypeptide lacking the amino acid substitution(s). In some embodiments, the recombinant IL-12p40 polypeptides have binding affinity for IL-12Rβ1 reduced by at least about 10%, about 20%, about 30%, about 40%, about 50%, about 60%, about 70%, about 80%, about 90%, or by at least about 95% compared to a reference IL-12p40 polypeptide lacking the amino acid substitution(s). In some embodiments, the recombinant IL-12p40 polypeptides have binding affinity for IL-12Rβ1 reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to IL-12Rβ1-binding affinity of a reference IL-12p40 polypeptide lacking the amino acid substitution(s). In some embodiments, the binding affinity of the IL-12p40 polypeptide variant of the disclosure to IL-12Rβ1 is determined by a Surface Plasmon Resonance (SPR) assay.

In some embodiments, the administered composition results in a reduced STAT4 signaling compared to a reference IL-12p40 polypeptide lacking the amino acid substitution(s). In some embodiments, STAT4 signaling in the subject is reduced by at least about 10%, about 20%, about 30%, about 40%, about 50%, about 60%, about 70%, about 80%, about 90%, or by at least about 95% compared to administration of a reference IL-12p40 polypeptide lacking the amino acid substitution(s). In some embodiments, STAT4 signaling in the subject is reduced by about 10% to about 100% compared to administration of a reference IL-12p40 polypeptide lacking the amino acid substitution(s). In some embodiments, STAT4 signaling in the subject is reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to administration of a reference IL-12p40 polypeptide lacking the amino acid substitution(s).

In some embodiments, the administered composition results in a reduced STAT3 signaling compared to administration of a reference IL-12p40 polypeptide lacking the amino acid substitution(s). In some embodiments, STAT3 signaling in the subject is reduced by at least about 10%, about 20%, about 30%, about 40%, about 50%, about 60%, about 70%, about 80%, about 90%, or by at least about 95% compared to administration of a reference IL-12p40 polypeptide lacking the amino acid substitution(s). In some embodiments, STAT3 signaling in the subject is reduced by about 10% to about 100% compared to administration of a reference IL-12p40 polypeptide lacking the amino acid substitution(s). In some embodiments, STAT4 signaling in the subject is reduced by about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to administration of a reference IL-12p40 polypeptide lacking the amino acid substitution(s).

In some embodiments, the administered composition results in a cell-type biased signaling of the downstream signal transduction mediated through IL-12p40 compared to a composition including a referenced IL-12p40 polypeptide lacking the amino acid substitution(s). In some embodiments, the administered composition results in a cell-type biased signaling of the downstream signal transduction mediated through IL-12 compared to a composition including a reference polypeptide lacking the amino acid substitution(s). In some embodiments, the administered composition results in a cell-type biased signaling of the downstream signal transduction mediated through IL-23 compared to a composition including a reference polypeptide lacking the amino acid substitution(s). In some embodiments, the administered composition results in a cell-type biased signaling of the downstream signal transduction mediated through IL-12 and IL-23 compared to a composition including a reference polypeptide lacking the amino acid substitution(s).

A In some embodiments, the administered composition results in a cell-type biased IL-12 signaling compared to a composition including a referenced IL-12p40 polypeptide lacking the amino acid substitution(s), wherein the cell-type biased signaling includes a reduced IL-12-mediated signaling in NK cells. In some embodiments, IL-12-mediated signaling in NK cells is reduced by at least about 10%, about 20%, about 30%, about 40%, about 50%, about 60%, about 70%, about 80%, about 90%, or by at least about 95% compared to a composition including a reference IL-12p40 polypeptide lacking the amino acid substitution(s). In some embodiments, the cell-type biased signaling includes a substantially unaltered IL-12 signaling in CD8+ T cells. In some embodiments, the administered composition results in an unaltered IL-12-mediated signaling in CD8+ T cells, e.g., the same or substantially the same IL-12-mediated signaling compared to a composition including a reference IL-12p40 polypeptide lacking the amino acid substitution(s). In some embodiments, the administered composition results in a reduced IL-12 signaling in NK cells while substantially retains IL-12 signaling in CD8+ T cells. In some embodiments, the administered composition substantially retains the recombinant polypeptide's capability to stimulate expression of INFγ in CD8+ T cells. In some embodiments, the administered composition enhances antitumor immunity in a tumor microenvironment.

In some embodiments, the subject is a mammal. In some embodiments, the mammal is a human. In some embodiments, the subject has or is suspected of having a condition associated with IL-12p40 mediated signaling. In some embodiments, the subject has or is suspected of having a condition associated with IL-12 mediated signaling. In some embodiments, the subject has or is suspected of having a condition associated with IL-23 mediated signaling. In some embodiments, the condition is a cancer, an immune disease, or a chronic infection.

The term cancer generally refers to the presence of cells possessing characteristics typical of cancer-causing cells, such as uncontrolled proliferation, immortality, metastatic potential, rapid growth and proliferation rate, and certain characteristic morphological features. Cancer cells are often observed aggregated into a tumor, but such cells can exist alone within an animal subject, or can be a non-tumorigenic cancer cell, such as a leukemia cell. Thus, the terms “cancer” or can encompass reference to a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

In some embodiments, provided herein are various methods for the treatment of a condition in a subject in need thereof, wherein the condition is a cancer selected from the group consisting of an acute myeloma leukemia, an anaplastic lymphoma, an astrocytoma, a B-cell cancer, a breast cancer, a colon cancer, an ependymoma, an esophageal cancer, a glioblastoma, a glioma, a leiomyosarcoma, a liposarcoma, a liver cancer, a lung cancer, a mantle cell lymphoma, a melanoma, a neuroblastoma, a non-small cell lung cancer, an oligodendroglioma, an ovarian cancer, a pancreatic cancer, a peripheral T-cell lymphoma, a renal cancer, a sarcoma, a stomach cancer, a carcinoma, a mesothelioma, and a sarcoma.

In some embodiments, the immune disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease. In some embodiments, the subject is a mammal. In some embodiments, the mammal is a human. In some embodiments, the subject has or is suspected of having a condition associated with perturbations in IL-12p40 mediated signaling. In some embodiments, the subject has or is suspected of having a condition associated with perturbations in IL-12 mediated signaling. In some embodiments, the subject has or is suspected of having a condition associated with perturbations in IL-23 mediated signaling.

### Additional Therapies

As discussed supra, any one of the recombinant polypeptides, nucleic acids, recombinant cells, cell cultures, and/or pharmaceutical compositions described herein can be administered in combination with one or more additional (e.g., supplementary) therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

In some embodiments, the methods of treatment as described herein further include administration of a compound that inhibits one or more immune checkpoint molecules. In some embodiments, the one or more immune checkpoint molecules include one or more of CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes. Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

In some aspects, the one or more anti-cancer therapies include radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of precancers or normal tissues is also contemplated herein.

Accordingly, in some embodiments, the composition is administered to the subject individually as a first therapy or in combination with a second therapy. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

Combination of IL-12/IL-23 Comprising IL-12p40 Variants with Supplementary Therapeutic Agents

The present disclosure provides for the use of IL-12 or IL-23 comprising a variant IL-12p40 subunit as described herein may be administered to a subject in combination with one or more additional active agents (“supplementary agents”). Such further combinations are referred to interchangeably as “supplementary combinations” or “supplementary combination therapy” and those therapeutic agents that are used in combination with IL-12 or IL-23 comprising a variant IL-12p40 subunit of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the IL-12p40 variants of the disclosure.

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g. IL-12 or IL-23 comprising a variant IL-12p40 subunit) is considered to be administered in combination with a second agent (e.g. a modulator of an immune checkpoint pathway) if the biological effect resulting from the administration of the first agent persists in the subject at the time of administration of the second agent such that the therapeutic effects of the first agent and second agent overlap. For example, the PD1 immune checkpoint inhibitors (e.g. nivolumab or pembrolizumab) are typically administered by I.V. infusion every two weeks or every three weeks while the IL-12 or IL-23 species comprising a variant p40 subunit of the present disclosure may be administered more frequently, e.g. daily, BID, or weekly. However, the administration of the first agent (e.g. pembrolizumab) provides a therapeutic effect over an extended time and the administration of the second agent (e.g., IL-12(p35/p40) variant or IL-23(p19/p40) variant) provides its therapeutic effect while the therapeutic effect of the first agent remains ongoing such that the second agent is considered to be administered in combination with the first agent, even though the first agent may have been administered at a point in time significantly distant (e.g., days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IL-12(p35/p40) variant or IL-23(p19/p40) variant polypeptide and the supplementary agent(s) are administered or applied sequentially, e.g., where one agent is administered prior to one or more other agents. In other embodiments, the IL-12(p35/p40) variant or IL-23(p19/p40) variant polypeptide and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the methods involving the combination of an IL-12(p35/p40) variant or IL-23(p19/p40) variant polypeptide and a supplementary agent which is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

Additional or Supplementary Agents

In some embodiments, the one or more additional (e.g., supplementary) therapeutic agent include a chemotherapeutic agent. In some embodiments, the supplementary agent is a “cocktail” of multiple chemotherapeutic agents. In some embodiments, the chemotherapeutic agent or cocktail is administered in combination with one or more physical methods (e.g., radiation therapy). The term “chemotherapeutic agents” includes, but is not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent isone or more chemical or biological agents identified in the art as useful in the treatment of neoplastic disease, including, but not limited to, a cytokines or cytokine antagonists such as IL-2, INFγ, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the IL-12(p35/p40) variant or (IL-23)p19/p40 variant is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

Combination with Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TriKE) constructs).

In some embodiments, the therapeutic antibody is an antibody that binds to at least one tumor antigen selected from the group consisting of HER2 (e.g., trastuzumab, pertuzumab, ado-trastuzumab emtansine), nectin-4 (e.g., enfortumab), CD79 (e.g., polatuzumab vedotin), CTLA4 (e.g., ipilumumab), CD22 (e.g. moxetumomab pasudotox), CCR4 (e.g. magamuizumab), IL23p19 (e.g., tildrakizumab), PDL1 (e.g., durvalumab, avelumab, atezolizumab), IL17a (e.g., ixekizumab), CD38 (e.g. daratumumab), SLAMF7 (e.g., elotuzumab), CD20 (e.g. rituximab, tositumomab, ibritumomab and ofatumumab), CD30 (e.g., brentuximab vedotin), CD33 (e.g., gemtuzumab ozogamicin), CD52 (e.g. alemtuzumab), EpCam, CEA, fpA33, TAG-72, CAIX, PSMA, PSA, folate binding protein, GD2 (e.g., dinuntuximab) , GD3, IL6 (e.g., silutxumab) GM2, Le^{y}, VEGF (e.g., bevacizumab), VEGFR, VEGFR2 (e.g., ramucirumab), PDGFRα (e.g., olartumumab), EGFR (e.g., cetuximab, panitumumab and necitumumab), ERBB2 (e.g., trastuzumab), ERBB3, MET, IGF1R, EPHA3, MUC-1, TRAIL R1, TRAIL R2, RANKL RAP, tenascin, integrin αVβ3, and integrin α4β1.

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies, CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies. Such therapeutic antibodies may be further conjugated to one or more chemotherapeutic agents (e.g., antibody drug conjugates or ADCs) directly or through a linker, especially acid, base or enzymatically labile linkers.

Combination with Physical Methods

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL-12(p35/p40) variant or IL23(p19/p40) variant and one or more supplementary agents. In some embodiments, the present disclosure further contemplates the use of an IL12p35/p40 variant or IL23p19/p40 variant in combination with surgery (e.g., tumor resection). In some embodiments, the present disclosure further contemplates the use of an IL-12p40 variant in combination with bone marrow transplantation, peripheral blood stem cell transplantation or other types of transplantation therapy.

Combination with Immune Checkpoint Modulators

In some embodiments, a supplementary agent is an immune checkpoint modulator for the treatment and/or prevention neoplastic disease in a subject as well as diseases, disorders or conditions associated with neoplastic disease. One skilled in the art will understand the term “immune checkpoint pathway” as a biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” As such, the term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

One skilled in the art will understand the term “immune checkpoint pathway modulator” as a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. One skilled in the art will understand the term “immune checkpoint pathway modulators” as encompassing both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”)

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the IL12p35/p40 variant or IL23p19/p40 variant is a positive immune checkpoint pathway agonist. In another embodiment, immune checkpoint pathway modulator employed in combination with an IL12p35/p40 variant or IL23p19/p40 variant is an immune checkpoint pathway antagonist.

One skilled in the art will understand the term “negative immune checkpoint pathway inhibitor” as an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

In some embodiments, PD1 pathway inhibitors include monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), and atezolizumab (Tecentriq®, Genentech/Roche, South San Francisco Calif.). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. Nos. 8,217,149; 8,168,757; 8,008,449; and 7,943,743.

PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130.).

In some embodiments, PD1 pathway inhibitors include peptidyl PD1 pathway inhibitors such as those described in Sasikumar, et al., U.S. Pat. No. 9,422,339; and Sasilkumar, et al., U.S. Pat. No. 8,907,053. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr. 16-20; New Orleans, La. Philadelphia (Pa.): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

In some embodiments, PD1 pathway inhibitors include small molecule PD1 pathway inhibitors. Examples of small molecule PD1 pathway inhibitors useful in the practice of the present invention are described in the art including Sasikumar, et al., 1,2,4-oxadiazole and thiadiazole compounds as immunomodulators (PCT/IB2016/051266, published as WO2016142833A1) and Sasikumar, et al. 3-substituted-1,2,4-oxadiazole and thiadiazole PCT/IB2016/051343, published as WO2016142886A2), BMS-1166 and Chupak L S and Zheng X. Compounds useful as immunomodulators. Bristol-Myers Squibb Co. (2015) WO 2015/034820 A1, EP3041822 B1; WO2015034820 A1; and Chupak, et al. Compounds useful as immunomodulators. Bristol-Myers Squibb Co. (2015) WO 2015/160641 A2. WO 2015/160641 A2, Chupak, et al. Compounds useful as immunomodulators. Bristol-Myers Squibb Co. Sharpe, et al. Modulators of immunoinhibitory receptor PD-1, and methods of use thereof, WO 2011082400 A2; and U.S. Pat. No. 7,488,802.

In some embodiments, combination of IL-12p35/p40 variant or IL-23p19/p40 variant and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of IL12p35/p40 variant or IL23p19/p40 variant and an PD1 immune checkpoint modulator is useful in the treatment of tumors characterized by high levels of expression of PDL1, where the tumor has a tumor mutational burden, where there are high levels of CD8+ T-cell in the tumor, an immune activation signature associated with IFNγ and the lack of metastatic disease particularly liver metastasis.

In some embodiments, the IL-12p35/p40 variant or IL-23p19/p40 variant is administered in combination with an antagonist of a negative immune checkpoint pathway that inhibits the binding of CTLA4 to CD28 (“CTLA4 pathway inhibitor”). Examples of CTLA4 pathway inhibitors are known in the art (See, e.g., U.S. Pat. Nos. 6,682,736; 6,984,720; and 7,605,238).

In some embodiments, the IL12p35/p40 variant or IL23p19/p40 variant is administered in combination with an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J. Biol. Sci. 8:1420-30).

In some embodiments, the IL-12p35/p40 variant or IL-23p19/p40 variant is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 A1 published Sep. 8, 2016 (F. Hoffman-LaRoche), Karunsky, U.S. Pat. No. 9,631,026; Karunsky, Sabatos-Peyton, et al. U.S. Pat. Nos. 8,841,418; 9,605,070; Takayanagi, et al., U.S. Pat. No. 8,552,156.

In some embodiments, the IL-12 or IL-23 comprising a variant p40 subunit is administered in combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392].

In some embodiments, the IL-12 or IL-23 comprising a variant p40 subunit is administered in combination with an A2aR inhibitor. A2aR inhibits T-cell responses by stimulating CD4+ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the IL12p35/p40 variants and IL23p19/p40 variants for use in the treatment disorders such as cancer and Parkinson's disease.

In some embodiments, the IL-12 or IL-23 comprising a variant p40 subunit is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration of a IL12p35/p40 variant or IL23p19/p40 variant in combination with an agent(s) that modulate at least one immune checkpoint pathway including immune checkpoint pathway modulators that modulate two, three or more immune checkpoint pathways.

In some embodiments the IL12p35/p40 variant or IL23p19/p40 variant is administered in combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of IL12p35/p40 variants and/or IL23p19/p40 variants in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the IL-12p35/p40 variant or IL-23p19/p40 variant may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL-12p35/p40 variant or IL-23p19/p40 variant of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

Cell Therapy Agents and Methods as Supplementary Agent

In some embodiments, the methods of the disclosure may include the combination of the administration of an IL-12(p35/p40) variant or IL-23(p19/p40) variant with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of a IL-12p40 variant as described herein.

CAR-T Cells

In some embodiments of the methods of the present disclosure, the supplementary agent is a “chimeric antigen receptor T-cell” (CAR-T cell) which generally refers to a T-cell that has been recombinantly modified to express a chimeric antigen receptor. One skilled in the art will understand that a chimeric antigen receptor (CAR) generally refers to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present invention are prepared in accordance with principles well known in the art. See e.g., Eshhaar et al. U.S. Pat. No. 7,741,465 B1; Sadelain, et al (2013) Cancer Discovery 3(4):388-398; Jensen and Riddell (2015) Current Opinions in Immunology 33:9-15; Gross, et al. (1989) PNAS USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

One skilled in the art will understand the term antigen binding domain (ABD) to refer to a polypeptide that specifically binds to an antigen expressed on the surface of a target cell. The ABD may be any polypeptide that specifically binds to one or more cell surface molecules (e.g., tumor antigens) expressed on the surface of a target cell. In some embodiments, the ABD is a polypeptide that specifically binds to a cell surface molecule associated with a tumor cell is selected from the group consisting of GD2, BCMA, CD19, CD33, CD38, CD70, GD2, IL3Rα2, CD19, mesothelin, Her2, EpCam, Muc1, ROR1, CD133, CEA, EGRFRVIII, PSCA, GPC3, Pan-ErbB and FAP. In some embodiments, the ABD is an antibody (as defined hereinabove to include molecules such as one or more VHHs, scFvs, etc.) that specifically binds to at least one cell surface molecule associated with a tumor cell (i.e. at least one tumor antigen) wherein the cell surface molecule associated with a tumor cell is selected from the group consisting of GD2, BCMA, CD19, CD33, CD38, CD70, GD2, IL3Rα2, CD19, mesothelin, Her2, EpCam, Muc1, ROR1, CD133, CEA, EGRFRVIII, PSCA, GPC3, Pan-ErbB and FAP. Examples of CAR-T cells useful as supplementary agents in the practice of the methods of the present disclosure include but are not limited to CAR-T cells expressing CARs comprising an ABD further comprising at least one of: anti-GD2 antibodies, anti-BCMA antibodies, anti-CD19 antibodies, anti-CD33 antibodies, anti-CD38 antibodies, anti-CD70 antibodies, anti-GD2 antibodies and IL3Rα2 antibodies, anti-CD19 antibodies, anti-mesothelin antibodies, anti-Her2 antibodies, anti-EpCam antibodies, anti-Mucl antibodies, anti-ROR1 antibodies, anti-CD133 antibodies, anti-CEA antibodies, anti-PSMA antibodies, anti-EGRFRVIII antibodies, anti-PSCA antibodies, anti-GPC3 antibodies, anti-Pan-ErbB antibodies, anti-FAP antibodies.

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

The IL-12(p35/p40) variant or IL-23(p19/p40) variant may be administered in combination with first, second, third or fourth generation CAR-T cells. The term first-generation CAR-T cell refers to a cell engineered to express a CAR wherein the cytoplasmic domain transmits the signal from antigen binding through only a single signaling domain, for example a signaling domain derived from the high-affinity receptor for IgE FcεR1γ or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CAR-T cell refers to a cell engineered to express a CAR that includes a co-stimulatory signal in addition to the CD3ζ signal. Coincidental delivery of the co-stimulatory signal enhances cytokine secretion and antitumor activity induced by CAR-transduced T-cells. The co-stimulatory domain is usually be membrane proximal relative to the CD3ζ domain. Third-generation CAR-T cell refers to a cell engineered to express a CAR that includes a tripartite signaling domain, comprising for example a CD28, CD3ζ, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand. Examples of intracellular signaling domains comprising may be incorporated into the CAR of the present invention include (amino to carboxy): CD3ζ; CD28-41BB-CD3ζ; CD28-OX40-CD3ζ; CD28-41BB-CD3ζ; 41BB-CD-28-CD3ζ and 41BB-CD3ζ.

The term includes CAR variants including but not limited split CARs, ON-switch CARS, bispecific or tandem CARs, inhibitory CARs (iCARs) and induced pluripotent stem (iPS) CAR-T cells. The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. The term “bispecific or tandem CARs” refers to CARs which include a secondary CAR binding domain that can either amplify or inhibit the activity of a primary CAR. The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility. The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Generally, the chimeric antigen receptor T-cells (CAR-T cells) are T-cells which have been recombinantly modified by transduction with an expression vector encoding a CAR in substantial accordance with the teaching above.

In some embodiments, the engineered T cell is allogeneic with respect to the individual that is treated. Graham et al. (2018) Cell 7(10) E155. In some embodiments an allogeneic engineered T cell is fully HLA matched. However not all patients have a fully matched donor and a cellular product suitable for all patients independent of HLA type provides an alternative.

If the T cells used in the practice of the methods of the disclosure are allogeneic T cells, such cells may be modified to reduce graft versus host disease. For example, the engineered cells of the present invention may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

In some embodiments the IL-12(p35/p40) variant or IL-23(p19/p40) variant is administered in combination with additional cytokines including but not limited to IL2, IL-7, IL-15 and IL-18 including analogs and variants of each thereof.

In some embodiments the IL-12(p35/p40) variant or IL-23(p19/p40) variant is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the IL-12(p35/p40) variants and IL-23(p19/p40) variants described herein include but are not limited to cyclosporin A (Shih, et al., (1989) Nature 339:625-626, IL-16 and analogs (including rhIL-16, Idziorek, et al., (1998) Clinical and Experimental Immunology 112:84-91), TGFb1 (Genesteir, et al., (1999) J Exp Med189(2): 231-239), and vitamin E (Li-Weber, et al., (2002) J Clin Investigation 110(5):681-690).

In some embodiments, the supplementary agent is an anti-neoplastic physical methods including but not limited to radiotherapy, cryotherapy, hyperthermic therapy, surgery, laser ablation, and proton therapy.

### Kits

Also provided herein are various kits for the practice of a method described herein. In particular, some embodiments of the disclosure relate to kits for methods of modulating IL-12p40-mediated signaling in a subject. Some other embodiments relate to kits for methods of treating a condition in a subject in need thereof. In some embodiments, a kit can include one or more of the recombinant IL-12p40 polypeptides, recombinant nucleic acids, recombinant cells, or pharmaceutical compositions as provided and described herein; and instructions for use thereof. For example, provided herein, in some embodiments, are kits that include one or more of: a recombinant polypeptide of the disclosure, an IL-12p40 polypeptide variant of the disclosure, a recombinant nucleic acid of the disclosure, a recombinant cell of the disclosure, or a pharmaceutical composition of the disclosure; and instructions for use thereof. In some embodiments, the kits of the disclosure can further include an IL-12p35 polypeptide, or nucleic acid encoding the IL-12p35 polypeptide. In some embodiments, the kits of the disclosure can further include an IL-23p19 polypeptide, or nucleic acid encoding the IL-23p19 polypeptide.

In some embodiments, the kits of the disclosure further include one or more syringes (including pre-filled syringes) and/or catheters (including pre-filled syringes) used to administer one any of the provided recombinant polypeptides, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the recombinant polypeptides.

In some embodiments, the components of a kit can be in separate containers. In some other embodiments, the components of a kit can be combined in a single container. For example, in some embodiments of the disclosure, the kit includes one or more of the recombinant IL-12p40 polypeptides, recombinant nucleic acids, recombinant cells, or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice a method described herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### General Experimental Procedures

### Human T Cell Signaling

For production of recombinant human IL-12 and IL-23, IL-12p40 (23-328) was cloned into pD649 vector with an N-terminal HA signal peptide and C-terminal AviTag (GLNDIFEAQKIEWHE, SEQ ID NO: 12) and 6× His. Human IL-12p35 (23-219) and IL-23p19 (28-189) were cloned into pD649 with an N-terminal HA signal peptide, Flag tag and TEV protease site. IL-12 (IL-12p35 and IL-12p40), IL-23 (IL-23p19 and IL-12p40) and IL-12p40 alone were expressed by transient transfection of Expi293F cells (ThermoFisher #A14527) according to manufacturer's protocols. Supernatants were subject to Ni-NTA purification and size exclusion chromatography (SEC).

For human T cell signaling, IL-12 and IL-23 variants were produced in Expi293 cells as described above. IL-12p35 and IL-23p19 were co-transfected with IL-12p40 variants (wild-type, E81A, F82A or P39A D40A E81A F82A) and purified by Ni-NTA followed by SEC. Human peripheral mononuclear cells (PBMCs) were isolated from Stanford Blood Bank samples using SepMate-50 columns (STEMCELL Technologies #85450) with Ficoll-Paque PLUS (GE Healthcare Cat #GE17-1440-02). Cells were diluted in sterile PBS (Gibco #20012-050) with 2% fetal bovine serum (FBS) and added to SepMate-50 columns pre-loaded with 15 ml Ficoll. Red blood cells were lysed using ACK lysis buffer (Gibco #A10492-01) for 5 min, quenched with PBS containing 2% FBS and resuspended at 50×10⁶/mL in freezing media containing 90% FBS and 10% DMSO. Cells were frozen overnight at −80° C. in a Mr. Frosty freezing container (ThermoFisher #5100-0001) and transferred to a −80° C. storage box for long term storage. Human PBMCs were stimulated in 6 well plates coated with 2.5 μg/mL αCD3 (OKT-3, BioLegend, #317326) in RPMI 1640-glutaMAX (Gibco #61870-127) with 10% FBS, non-essential amino acids (Gibco #11140050), sodium pyruvate (Gibco Cat #11360-070), 15 mM HEPES (Gibco #15630-080) and penicillin-streptomycin (Gibco Cat #15140163) supplemented with 5 μg/mL αCD28 (CD28.2, BioLegend, #302943) and 100 IU/mL recombinant human IL-2. Cells were cultured for 48 hours at 37° C. with 5% CO₂, cells were washed once and rested overnight in complete RPMI. Cells were stained with αCD4 PacBlue (RPA-T4, BD, #558116) and stimulated with IL-12 and IL-23 variants for 20 minutes at 37° C. prior to fixation with 1.6% paraformaldehyde for 10 minutes at room temperature and permeabilization with methanol at −20° C. Cells were washed in PBS with 2% FBS and 2 mM EDTA and stained with antibodies against STAT4 pY693 AF488 (38/p-Stat4, BD, #558136) and STAT3 pY705 AF647 (4/P-STAT3, BD, #557815) for 1 hour at room temperature. Fluorescence intensity was analyzed using a CytoFlex flow cytometer (Beckman Coulter).

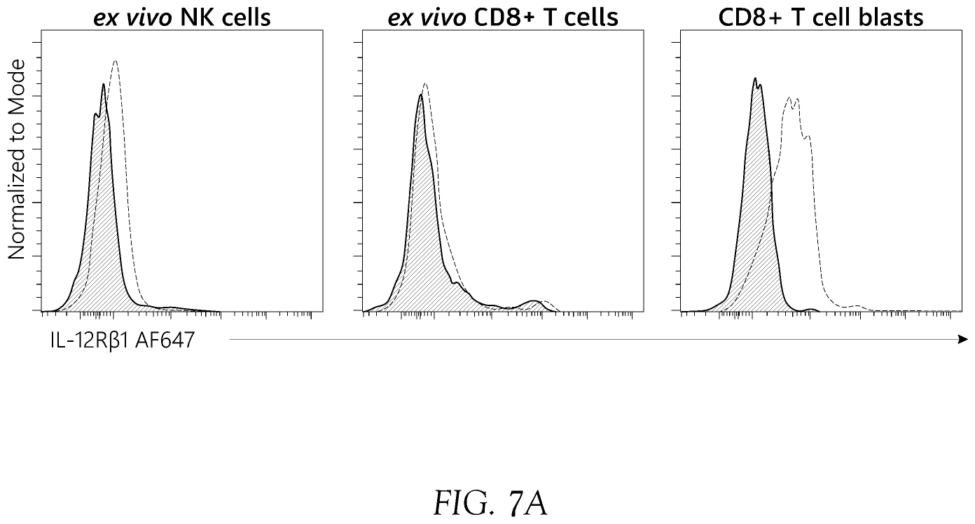

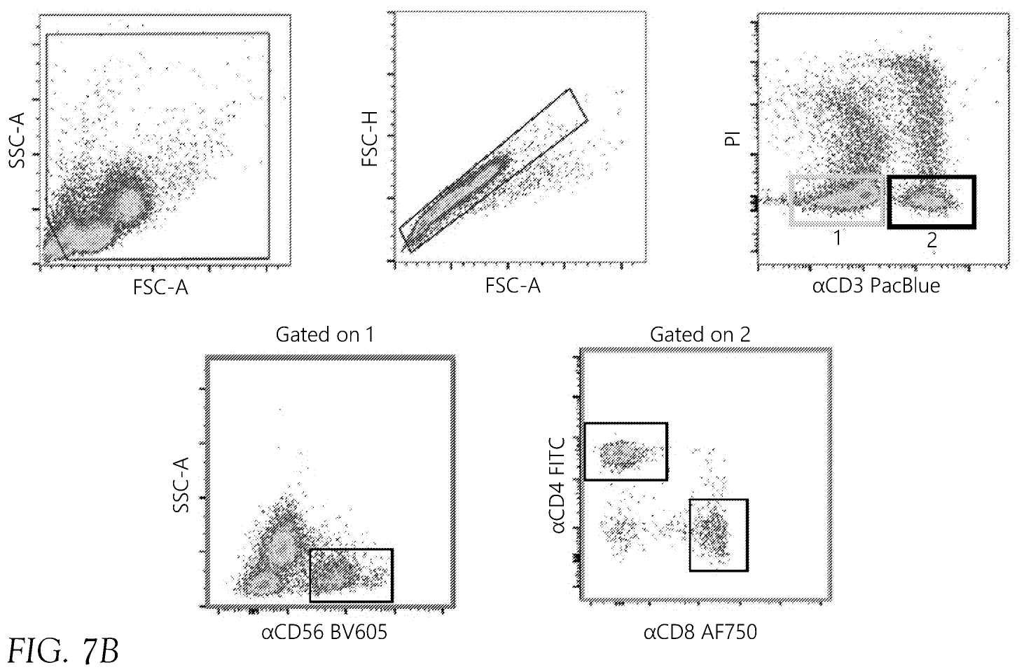

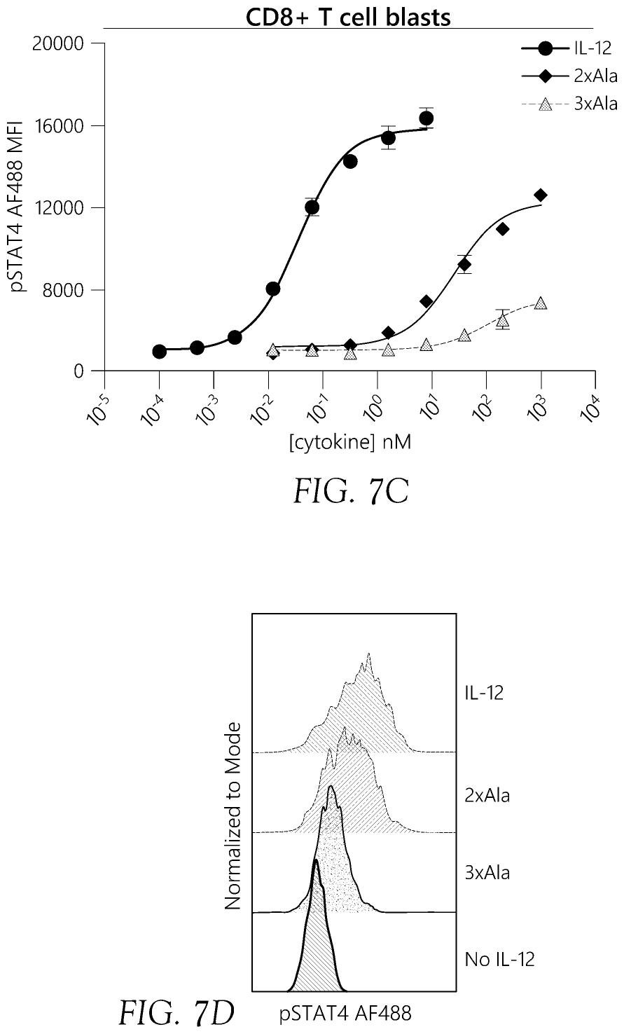

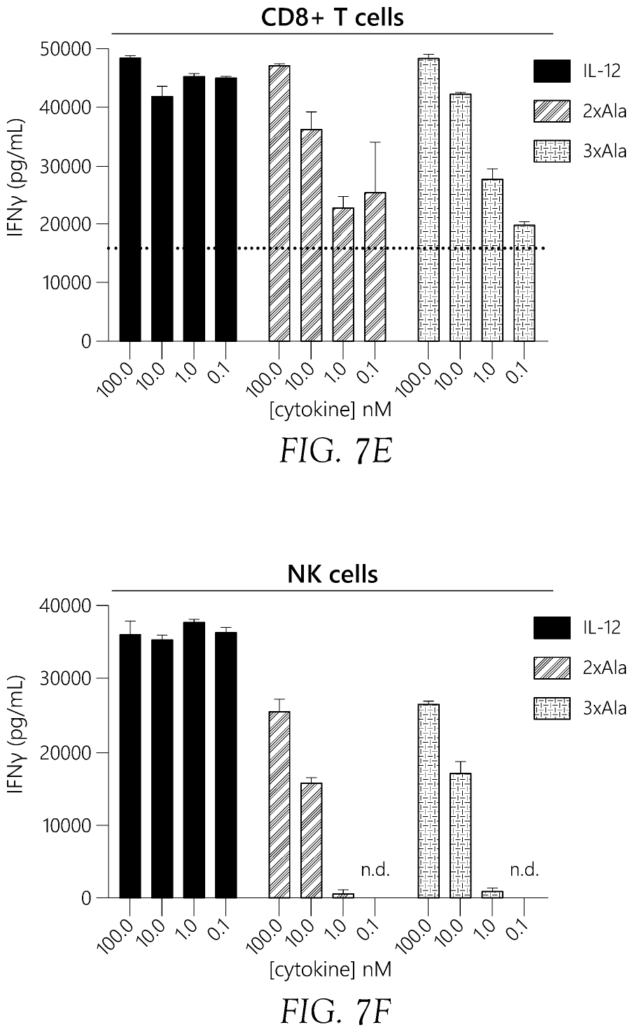

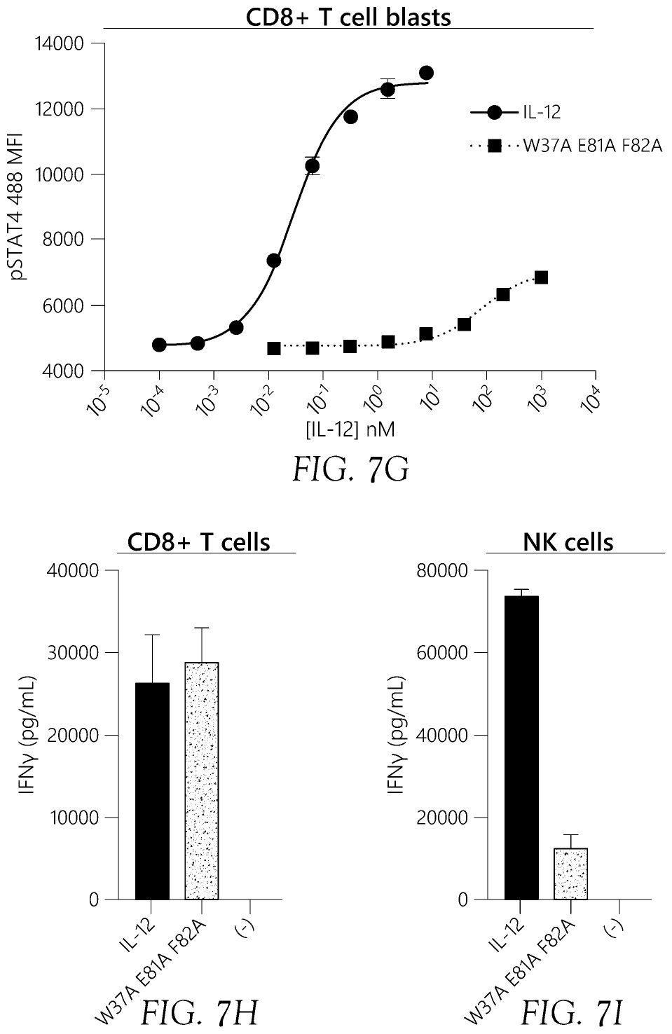

For analysis of IL-12Rβ1 in human PBMCs, cells were stained directly (ex vivo) or activated as described above to generate T cell blasts. To identify T cells and NK cells, Fc receptors were blocked with TruStain FcX (BioLegend) and cells were stained with a phenotyping panel of αCD3 Pacific Blue (UCHT1, BioLegend), αCD4 FITC (OKT4, BioLegend), αCD8 AF750 (R&D systems), and αCD56 BV605 (HCD56, BioLegend). Human p40 tetramers were prepared by mixing 200 nM streptavidin-AF647 with four-fold molar excess of biotinylated p40 expressed as described in the surface plasmon resonance section. Cells were stained for 2 hours at 4° C. followed by live cell detection using propidium iodide (PI, Invitrogen). Samples were analyzed using CytoFlex flow cytometer (Beckman Coulter) followed by analysis in FlowJo (BD). CD8⁺ T cells were defined as liveCD3+CD8+, NK cells were defined as liveCD3−CD56+. See FIG. 7B for gating.

For human CD8⁺ T cell IFNγ induction assay, CD8⁺ T cells were isolated from PBMCs by MACS using CD8⁺ T cell isolation kit (Milteny) and LS magnetic columns (Miltenyi). Purified CD8⁺ T cells were stimulated at 80,000 cells/well in 96-well round bottom plates in coated with 2 μg/mL αCD3 (OKT3, BioLegend) in the presence of 0.5 μg/mL αCD28 (CD28.2, BioLegend), and 5 ng/mL human IL-2. After 48 hours, cells were pelleted and supernatant was analyzed using human IFNγ ELISA MAX Deluxe (BioLegend) with Nunc MaxiSorp ELISA plates (BioLegend). For human NK cell IFNγ induction assays, NK cells were isolated from PBMCs by MACS using the EasySep human NK cell isolation kit (StemCell) with EasySep Magnet (StemCell). Purified NK cells were stimulated at 40,000 cells/well in 96 well round bottom plates in the presence of 100 ng/mL IL-18 (R&D systems). After 48 hours, supernatant was harvested and processed as described for CD8⁺ T cell IFNγ induction assays.

### IL-12p40 Surface Staining

For mIL-12p40 surface staining, mouse IL-12p40 (23-335) was cloned into pAcGP67a with N-terminal GP64 signal peptide and C-terminal AviTag and 6× His tags. Mouse IL-12p40 is secreted as a disulfide bonded homodimer so in order to obtain monomeric IL-12p40, Ni-NTA purified protein was reduced with 20 mM cysteine and alkylated with 40 mM iodoacetamide in HEPES buffered saline (HBS) pH 8.2 followed by SEC. Monomeric IL-12p40 was biotinylated with recombinant BirA and purified by a second round of SEC.

Spleen and lymph nodes from C57/BL6 mice were isolated and single cell suspension was generated. T cell blasts were activated on plates coated with 2.5 μg/mL αCD3 (145-2c11, BioLegend, Cat #100340) in complete RMPI with 5 μg/mL αCD28 (37.51, Bio X Cell, Cat #BE0015-1) and 100 IU/mL recombinant mouse IL-2 for 48 hours at 37° C. For cell staining, ex vivo cells and T cell blasts were incubated with TruStain FcX (93, BioLegend, 101320) and stained with a phenotyping panel of αCD3 FITC (17A2, eBiosciences, #11-0032-82), αCD4 PerCP-Cy5.5 (GK1.5, BioLegend, #100433), αCD8 BV785 (53-6.7, Biolegend, #100749) and αNK1.1 e450 (PK136, eBioscience, #48-5941-82). IL-12p40 tetramers were prepared by mixing 200 nM Streptavidin-AF647 with four-fold molar excess of biotinylated IL-12p40 and cells were stained for 2 hours at 4° C. followed by live cell staining with propidium iodide (PI, ThermoFisher #P3566). Samples were analyzed using CytoFlex flow cytometer followed by analysis in FlowJo. CD8+ T cells were defined as liveCD3+CD8+, NK cells were defined as liveCD3-NK1.1+.

### Mouse IL-12 Signaling

For IL-12 signaling and functional assays, mouse IL-12 was expressed as a single chain, similar to a previously described approach (Anderson et al., 1997). Mouse IL-12p40 (23-335) followed by a 3×GGGS linker, 3C protease site and mouse IL-12p35 (23-215) was cloned into pAcGP67a with an N-terminal GP64 signal peptide and C-terminal 6× His tag. Mouse IL-12 variants were expressed in T. ni cells and purified by Ni-NTA and SEC. For cell signaling, mouse T cell blasts were prepared as described above, rested overnight in complete RPMI, stained with αCD8 BV785 (53-6.7, Biolegend, #100749) and stimulated for 20′ at 37° C. with IL-12 variants before fixation, permeabilization and staining for pSTAT4 as described for human T cell signaling.

### NK Cell INFγ Induction

For NK cell IFNγ induction assays, NK cells were isolated from spleen and lymph nodes of C57/BL6 mice using the mouse NK cell isolation kit (Miltenyi #130-115-818) and LS magnetic columns (Mitenyi #130-042-401). NK cells were stimulated at 25,000 cells/well in a 96 well round bottom plate with 50 ng/mL recombinant mouse IL-18 (R&D systems #9139-IL-010) and 1 μM IL-12 variants for 48 hours at 37° C. In the final four hours of culture, GolgiStop (BD #554724) was added to prevent further cytokine secretion. Cells were fixed and permeabilized using Cytofix/Cytoperm kit (BD, #554714) and stained with αIFNγ AF647 (XMG1.2, BD, #557735). Fluorescence intensity was recorded using CytoFlex flow cytometer and analyzed in FlowJo.

### CD8+ T Cell IFNγ Induction

For CD8+ T cell effector assays, OT-I TCR transgenic mice (C57BL/6-Tg(TcraTcrb)1100Mjb/j) (Hogquist et al., 1994) were obtained from Jackson Labs and maintained in the Stanford animal facility according to protocols approved by the Stanford University Institutional Animal Care and Use Committee. OT-I splenocytes were stimulated in media containing 1 μg/mL ovalbumin (aa257-264, GenScript #RP10611), 100 IU/mL rmIL-2 and 1 μM IL-12 variants. For IFNγ induction assays, cells were stimulated for 48 hours at 80,000 cells/well in a 96 well round bottom plate. For the final four hours, GolgiStop was added to prevent further cytokine secretion. Cells were stained with αCD3 e450 (17A2, eBioscience, 48-0032-82) and αCD8 BV785 (53-6.7, Biolegend, #100749) before being fixed/permeabilized using the Cytofix/Cytoperm kit and stained with αIFNγ AF647. Samples were gated on CD3+CD8+ cells and αIFNγ AF647 staining was assessed using CytoFlex flow cytometer followed by analysis in FlowJo.

### MHC-I Upregulation

For MHC-I upregulation, 25,000 B16F10 melanoma cells (ATCC #CRL-6475) were plated on 96 flat bottom plates for 4 hours at 37° C. Supernatant from OT-I effectors, generated with or without IL-12 variants as described above, were diluted in media and added to B16F10 cells for 16 hours at 37° C. Following overnight incubation, media was removed and B16F10 cells were detached using TrypLE (ThermoFisher #12604013). Cells were stained with αH-2K^{b} APC (AF6-88.5.5.3, BioLegend, #116512) and PI to identify live cells. Data were collected on CytoFlex flow cytometer and analyzed in FlowJo.

### Antigen-Specific Tumor Cell Killing

For antigen-specific tumor cell killing, B16F10 cells were transduced with pCDH-EF1-cOVA-T2A-copGFP (Tseng et al., 2013) and sorted to obtain a pure population of OVA-GFP expressing cells. B16F10 wild-type and OVA-GFP were mixed at a 1:1 ratio and 25,000 cells were plated on a 96 flat bottom plate. After 4 hours at 37° C., media was removed and OT-I effectors, generated with or without IL-12 variants as described above, were added in complete RPMI for 36 hours at 37° C. Media was removed and B16F10 were detached using TrypLE, stained with PI and αCD45.2 APC (104, eBioscience, #17-0454-82) prior to running samples on CytoFlex. B16F10 were identified as liveCD45.2− and % GFP+ was quantified as compared to no effector condition.

### Example 2

### Crystal Structure of IL-12Rβ1 and the Quaternary IL-23 Receptor Complex

This Example describes the results of experiments performed to determine the crystal structure of IL-12Rβ1 and the quaternary IL-23 receptor complex, which in turns helps elucidate the chemistry that drives each of the cytokine-receptor interactions of the heteromeric receptor complex.

As described above, IL-23(IL-23p19/IL-12p40) signals through a receptor complex composed of IL-23R and IL-12Rβ1 (FIG. 1A). The ECD of IL-12Rβ1 consists of 5 fibronectin type III (FNIII) domains of which the N-terminal D1-D2 domains mediate binding to IL-23. Experiments were designed and performed to crystalize a complex of IL-12Rβ1 D1-D2 with IL-23 and the IL-23R ectodomain. Table 3 below summarizes the crystallographic data and refinement statistics of the quaternary complex diffracted to 3.4 Å resolution.

A structure of part of the complex was determined by molecular replacement using the previously published IL-23R ternary (IL-23p19/IL-12p40/IL-23R) complex. However, the structure of IL-12Rβ1 was still needed. Thus, additional experiments were performed to determine the structure of the human IL-12Rβ1 D1-D2 domains to a resolution of 2.0 Å using single isomorphous replacement with anomalous scattering (SIRAS). Subsequently, this newly established structure was used as a search model that allowed for placing the IL-12Rβ1 D1 domain in the electron density of the quaternary complex. The D2 domain was not visible which is likely due to flexibility in the crystal lattice.

It was observed that the quaternary IL-23 receptor complex exhibits a modular architecture in which IL-23 serves as a bridge to coalesce IL-23R and IL-12Rβ1 and initiate JAK1/Tyk2 trans-phosphorylation inside the cell (FIGS. 1B-1E). A summary of IL-12p40 and IL-12Rβ1 contacts is provided in Table 3 below.

TABLE 3
IL-12p40 and IL-12Rβ1 contacts from PISA.

| P40 residue | IL-12Rβ1 residue | Type | Mainchain/sidechain |
|---|---|---|---|
| Trp 37 | Leu 108 | vdw | sc-sc |
| Pro 39 | Asn 135 | vdw | sc-sc |
| | Tyr 134 | vdw | sc-sc |
| Asp 40 | Leu 108 | hb | mc-mc |
| | Gln 132 | hb | sc-sc |
| Ala 41 | Tyr 109 | hb | sc-sc |
| Lys 80 | Tyr 109 | vdw | mc-sc |
| Glu 81 | Ser 106 | vdw | sc-sc |
| Phe 82 | Asp 101 | vdw | sc-mc |
| | Gln 102 | vdw | sc-sc |
| Glu 108 | Tyr 134 | vdw | sc-sc |
| | | hb | sc-sc |
| Asp 115 | Asp 58 | vdw | sc-sc |
| His 216 | Gln 102 | hb | mc-sc |
| | | | sc-sc |
| Lys 217 | Gln 102 | hb | sc-sc |
| Leu 218 | Gln 102 | hb | mc-sc |
| Lys 219 | Asp 58 | hb | sc-sc |
| | Asp 101 | hb | sc-sc |
| | Gln 102 | vdw | sc-sc |

Abbreviations are as follows: vdw, van der Waals; hb, hydrogen bond; sc, side chain; mc main chain.

The shared receptor, IL-12Rβ1 binds at the “back” of IL-12p40 at the intersection between the D1 N-terminal Ig and D2 fibronectin domains IL-12p40 (FIG. 1D). The D1 domain of IL-12p40 is tilted forward relative to the D2 domain, exposing a cleft between the base of D1 and the top of D2 to form a docking site for IL-12Rβ1. The D1 domain of IL-12Rβ1 binds IL-12p40 in a single, 1425 Å² interface that is characterized by a high degree of charge complementarity between the interacting proteins. The base of the interface is formed by a contiguous, positively charged loop in IL-12p40 (His216, Lys217 and Lys219) which interacts with a negatively charged patch in IL-12Rβ1 made up of Glu28, Asp58 and Asp101. Above these charge-charge interactions sits a hydrophobic strip on IL-12p40 formed by the aromatic residues, Tryp37 and Phe82 that is ringed by polar residues in IL-12Rβ1 (Glu102, Ser106, Tyr109, Gln132 and Tyr134) that make hydrogen bonding interactions with side chain and main chain atoms in IL-12p40.

### Example 3

### IL-12p40 Acts as a Common Regulator of IL-12 and IL-23 Signaling

This Example describes experiments performed to demonstrate that IL-12p40 acts as a common regulator of IL-12 and IL-23 signaling.

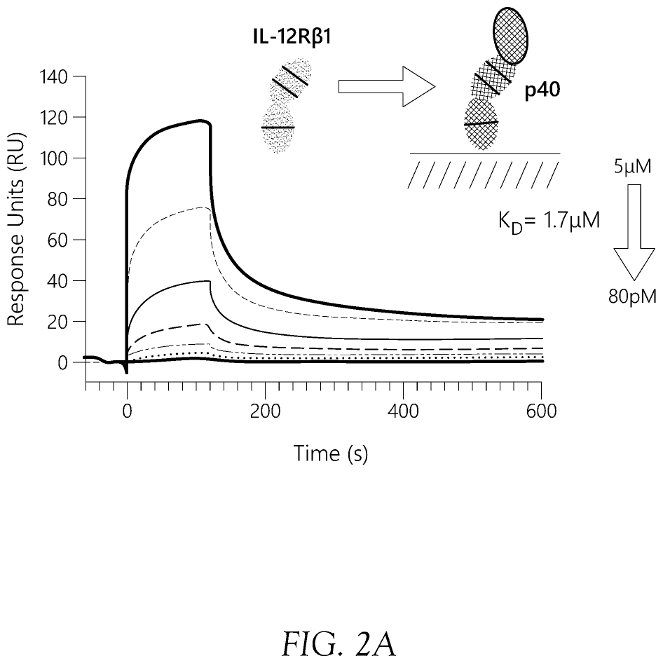

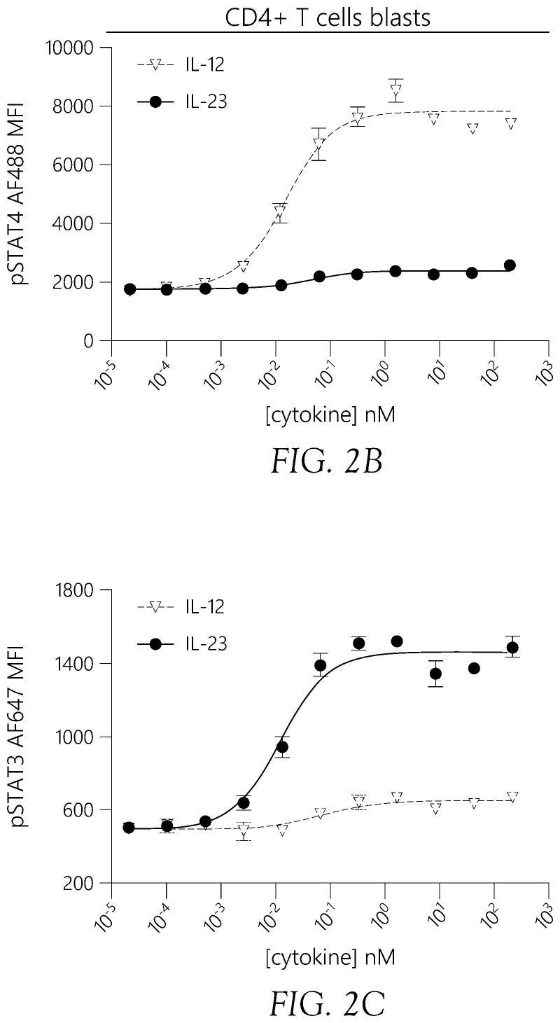

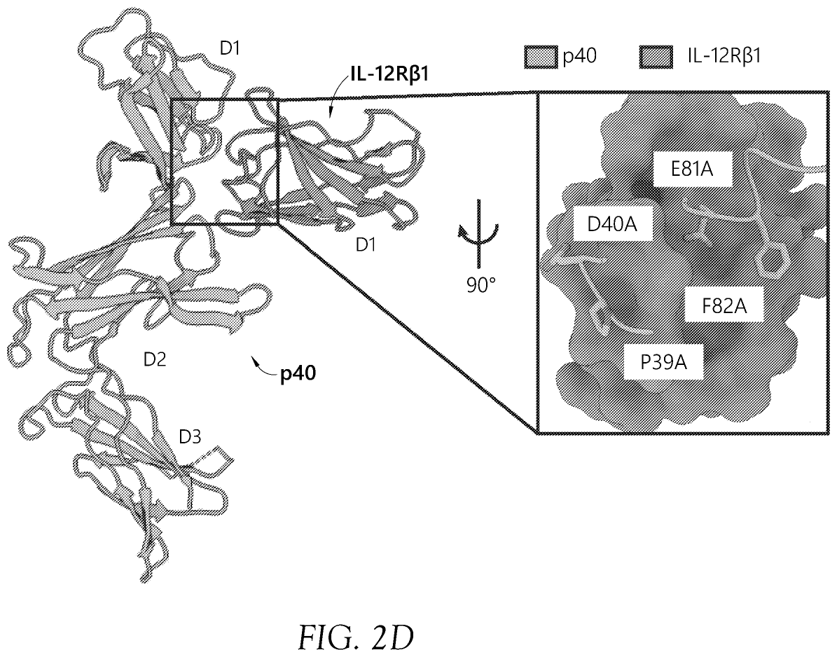

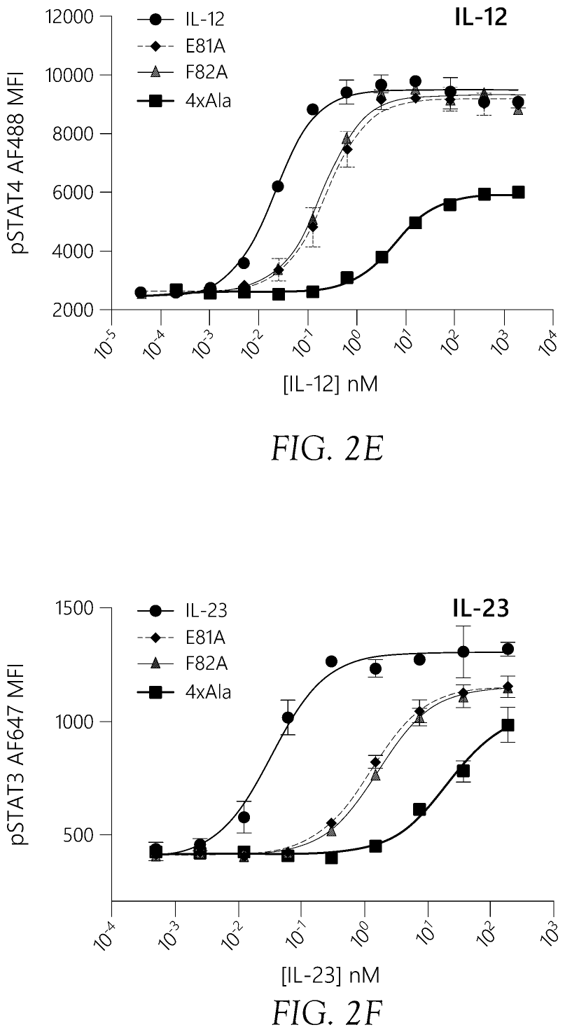

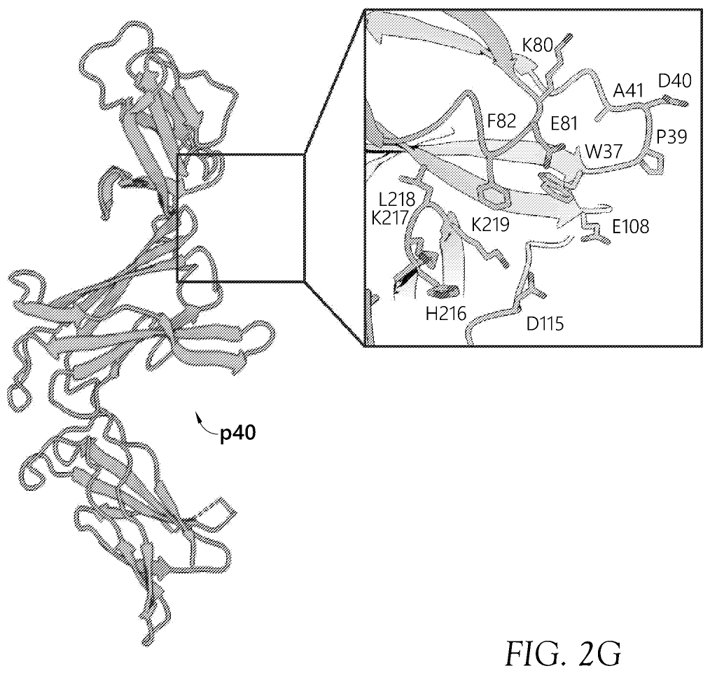

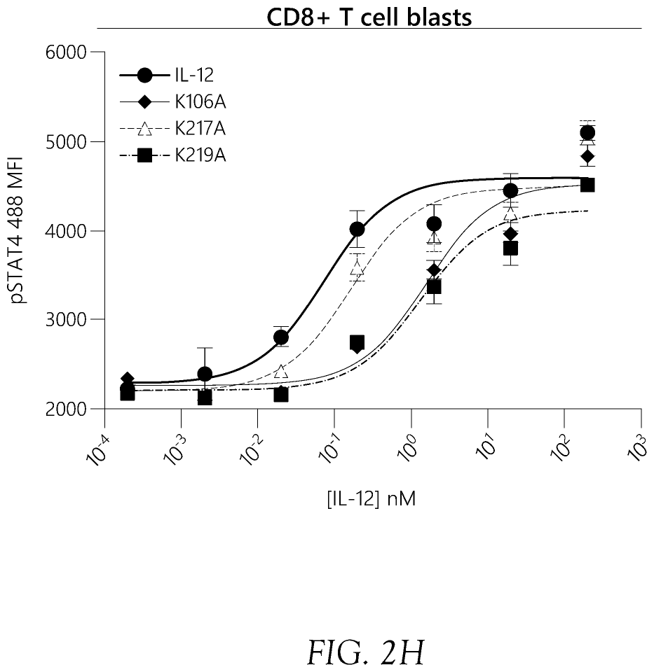

The IL-23 receptor complex crystal structure described in Example 2 above revealed that IL-12p40 directly engages IL-12Rβ1, indicating that IL-12p40 may play a conserved role in IL-12 and IL-23 signaling. This was confirmed by surface plasmon resonance (SPR) binding measurements which show that IL-12Rβ1 binds IL-12p40 with an affinity of 1.7 μM (FIG. 2A). To explore differences in IL-12 and IL-23 signaling, several experiments have been designed and performed to stimulate human CD4+ T cells with IL-12 or IL-23, as well as to measure phosphorylation of STAT3 and STAT4 by phospho-flow cytometry. It was observed that IL-12 stimulation preferentially resulted in the phosphorylation of STAT4 while IL-23 more strongly promoted STAT3 phosphorylation (FIGS. 2B-2C).

Based on the shared role of the IL-12p40/IL-12Rβ1 interaction in both the IL-12 and IL-23 receptor complexes as discussed above, additional experiments have been designed and performed to target this interface to modulate the level of STAT4 signaling in the context of IL-12, and STAT3 signaling in the context of IL-23, by ‘tuning’ the efficiency of IL-12Rβ1 recruitment. In particular, a panel of IL-12 and IL-23 partial agonists were created by introducing alanine substitutions in two loops of IL-12p40 D1 that mediate interactions with IL-12Rβ1 (FIG. 2D). In these experiments, it was found that individual alanine mutations (E81A and F82A) reduced the potency of IL-12 and IL-23, as indicated by a right shift in the dose-response curves for pSTAT4 and pSTAT3 (FIGS. 2E-2F). In these experiments, a greater increase in cytokine EC₅₀ and a reduced maximal STAT phosphorylation was obtained by combing multiple alanine mutations (4×Ala: P39A/D30A/E81A/F82A).

The complete list of IL-12p40 amino acid positions that engage IL-12Rβ1 is shown in FIG. 2G and IL-12 signaling of additional alanine mutations is shown in FIG. 2H.

### Example 4

### IL-12 Partial Agonists Elicit Cell-Type Specific Activity Based on Differential IL-12Rβ1 Expression

This Example describes the results from experiments performed with murine IL-12 to demonstrate that IL-12 partial agonists elicit cell-type specific activity based on differential IL-12Rβ1 expression.

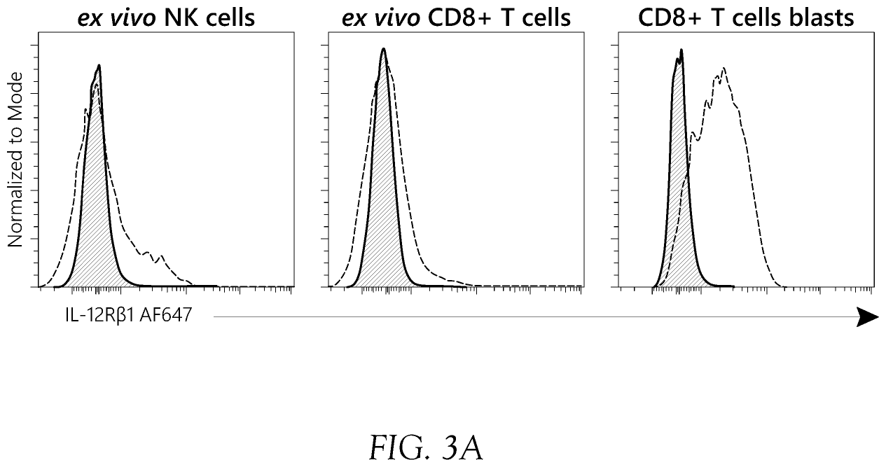

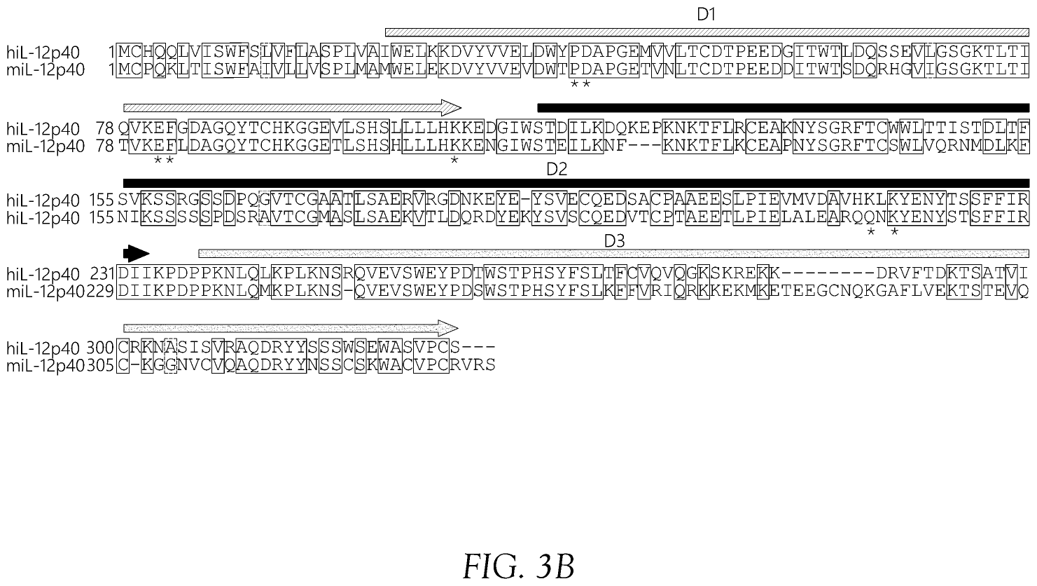

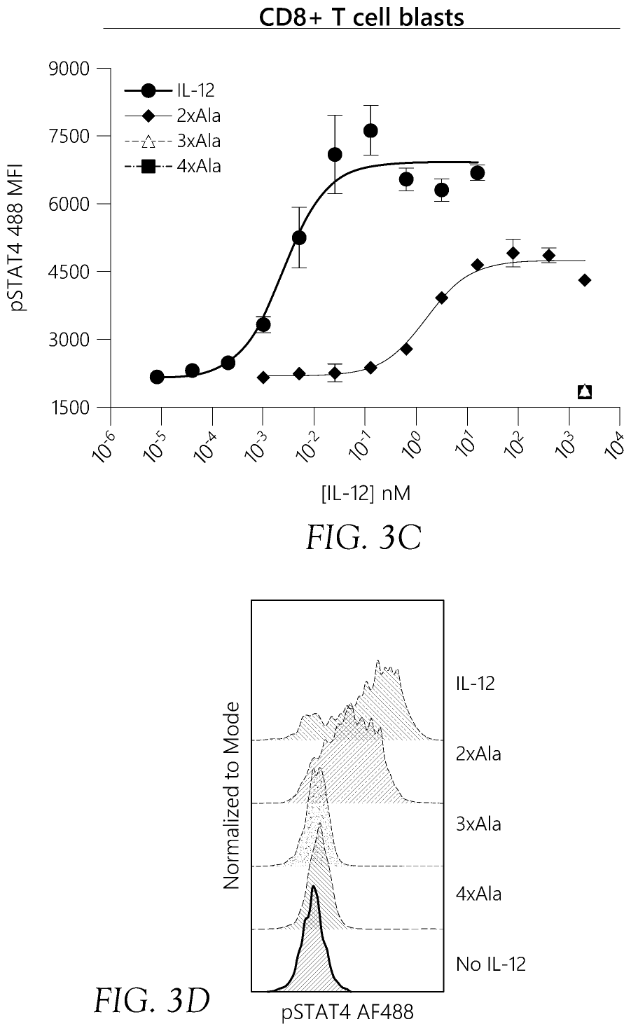

As discussed above, systemic administration of IL-12 often leads to toxicity due to NK cell mediated IFNγ production. Thus, biasing IL-12 signal to preferentially activate T cells, but with reduced NK cell IFNγ induction may reduce toxicity. An important difference in IL-12 signaling between T cells and NK cells is that antigen stimulation through the T cell receptor enhances IL-12 sensitivity through upregulation of its receptor subunits. Using IL-12p40 as a FACS staining reagent to assess IL-12Rβ1 surface expression, it was found that murine CD8+ T cell blasts have higher IL-12Rβ1 expression than NK cells or ex vivo CD8+ T cells (FIG. 3A).

As the structure has shown, IL-12p40 mediates recruitment of IL-12Rβ1. Accordingly, without being bound to any particular theory, it was hypothesized that reducing the affinity of IL-12p40 for IL-12Rβ1 may more severely impair signaling on NK cells which have reduced levels of IL-12Rβ1 expression relative to antigen experienced T cells. Additional experiments were designed and performed to design a series of partial agonist alanine mutations in murine IL-12p40 that would be predicted to disrupt binding to IL-12Rβ1 based on sequence homology with human IL-12p40 (FIG. 3B). To characterize mouse IL-12 variants, experiments were performed to test signaling on CD8+ T cell blasts. As predicted, it was found that mutations in IL-12p40 at the IL-12Rβ1 binding interface increased the EC50 and reduced the maximal STAT4 phosphorylation with (3× Alanine) and (4× Alanine) mutants did not inducing measurable STAT4 phosphorylation in this acute signaling assay (FIG. 3C).

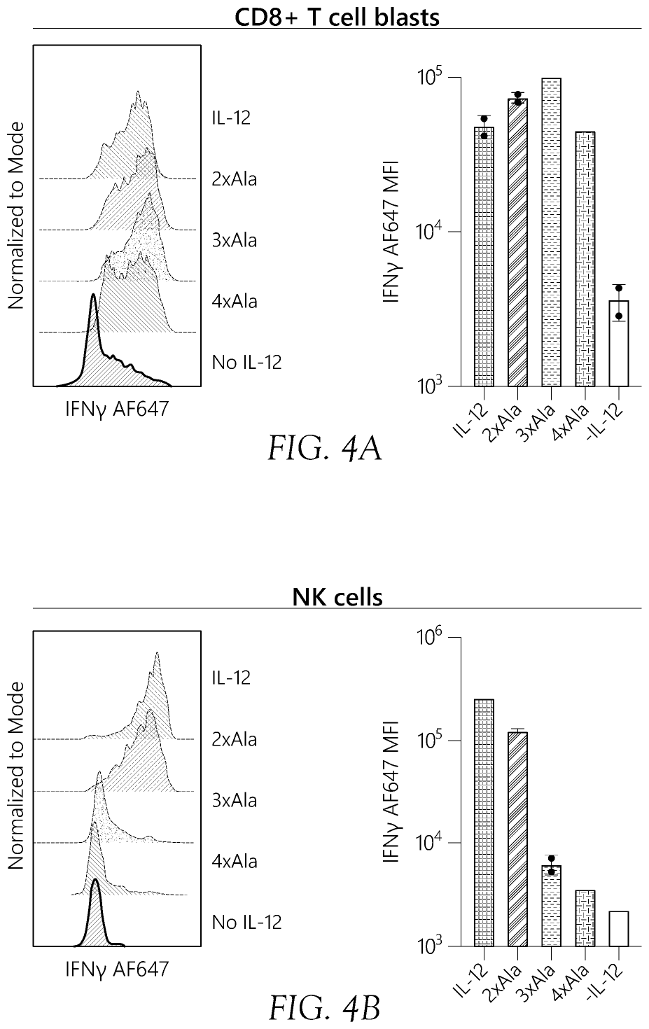

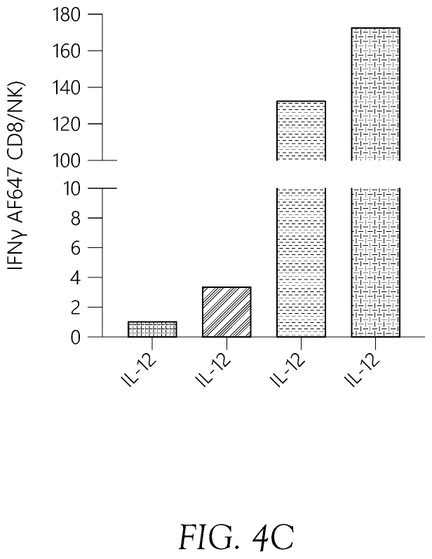

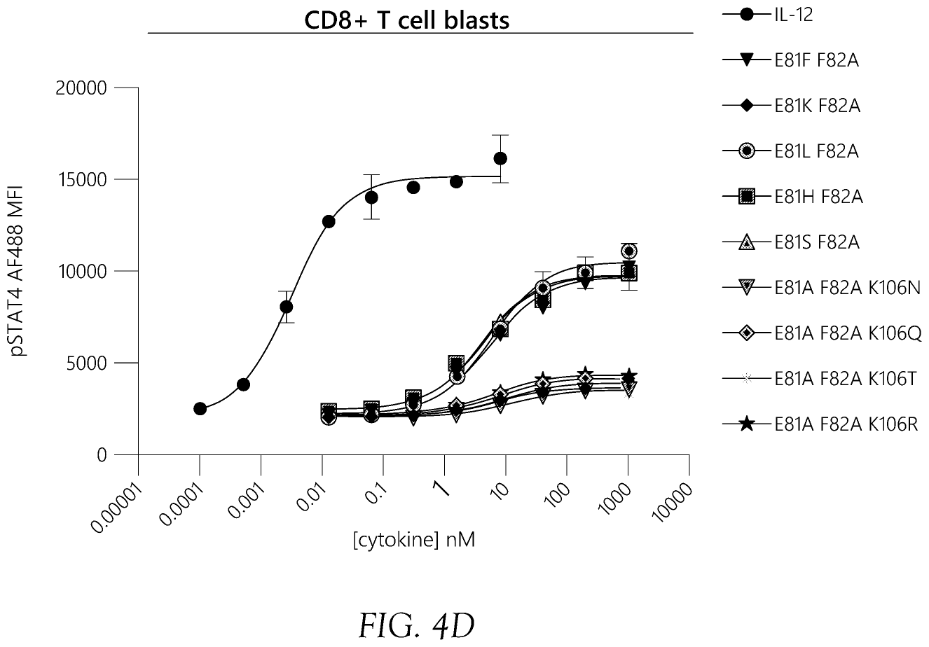

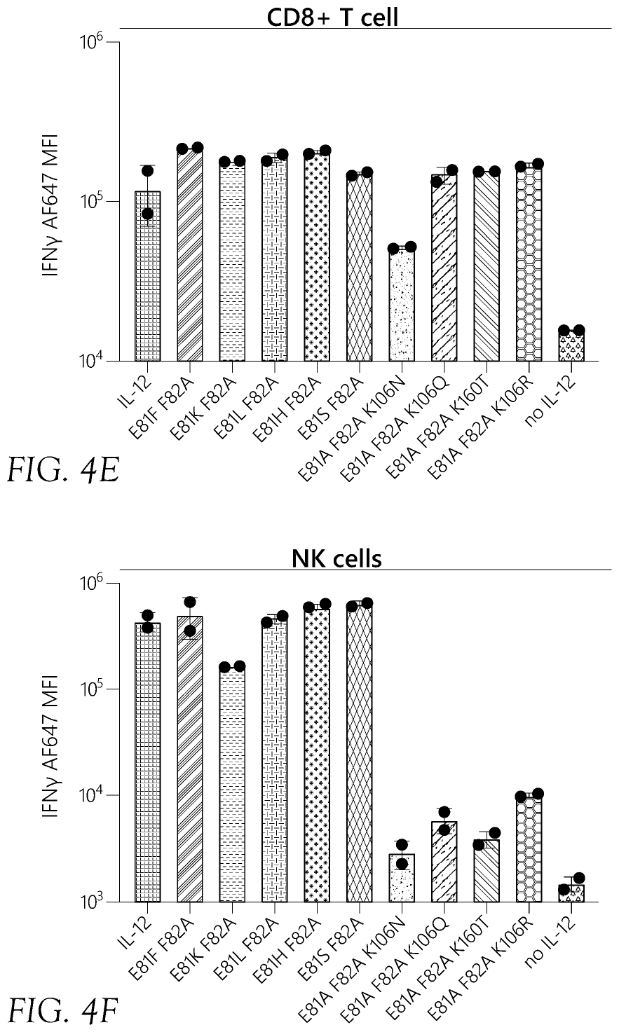

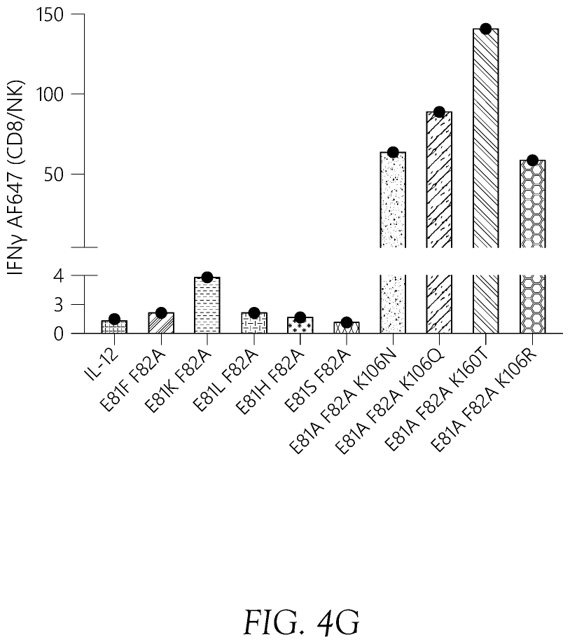

A well-documented output of IL-12 signaling in both T cells and NK cells is the induction of IFNγ. To determine the capacity of IL-12 partial agonists to promote IFNγ production in antigen-specific CD8+ T cells, additional experiments were performed to stimulate ovalbumin specific OT-I T cell (Hogquist et al., 1994) with OVA peptide and IL-12 variants for 48 hours before assessing IFNγ production by intracellular cytokine stain. IL-12 along with the 2×, 3×, and 4× alanine variants lead to upregulation of IFNγ despite the fact that the 3×Ala and 4×Ala mutants do not produce measurable STAT4 phosphorylation upon acute stimulation (FIG. 4A). This discrepancy may be due to differences in sensitivity or the greater time for signal integration between the assays.

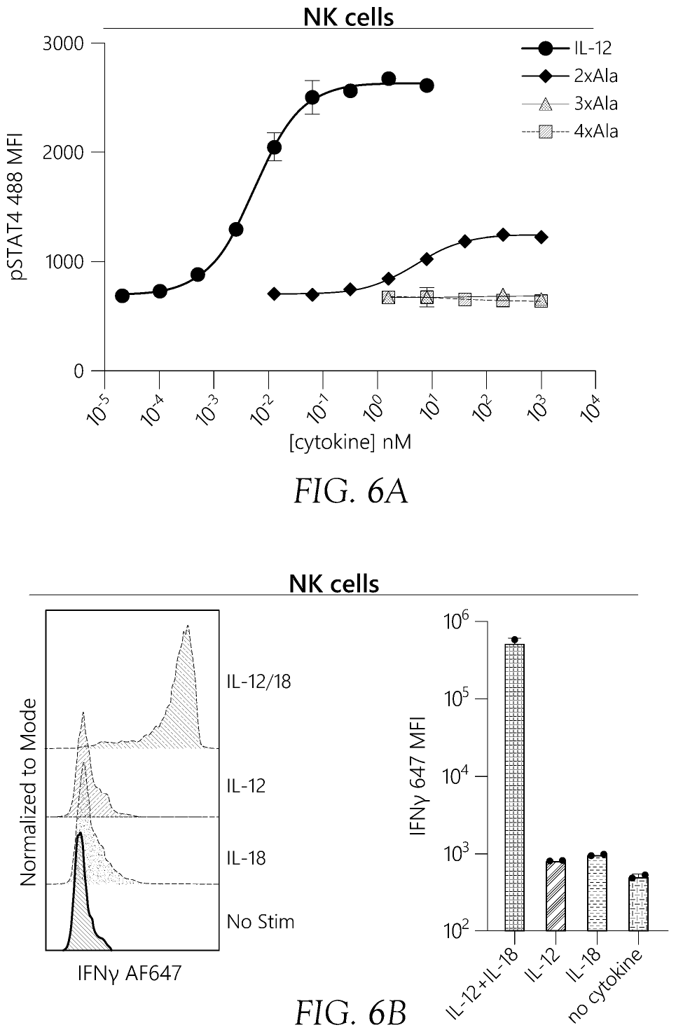

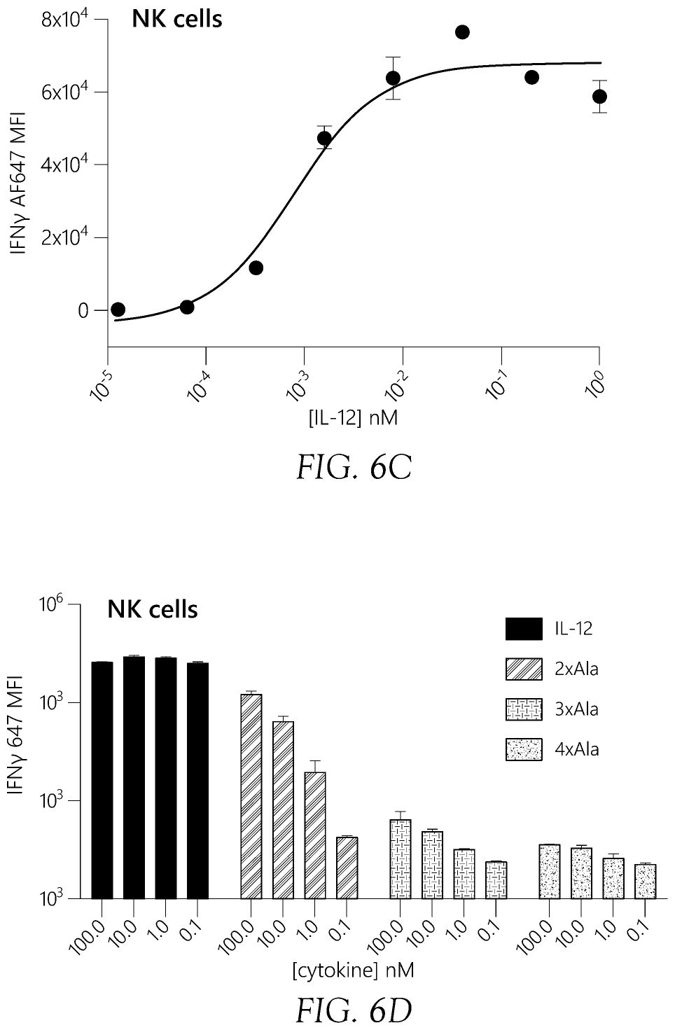

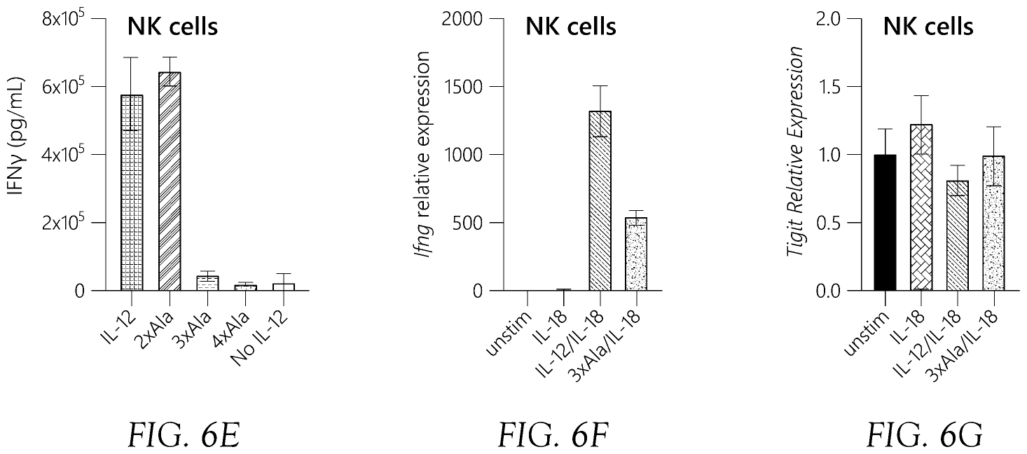

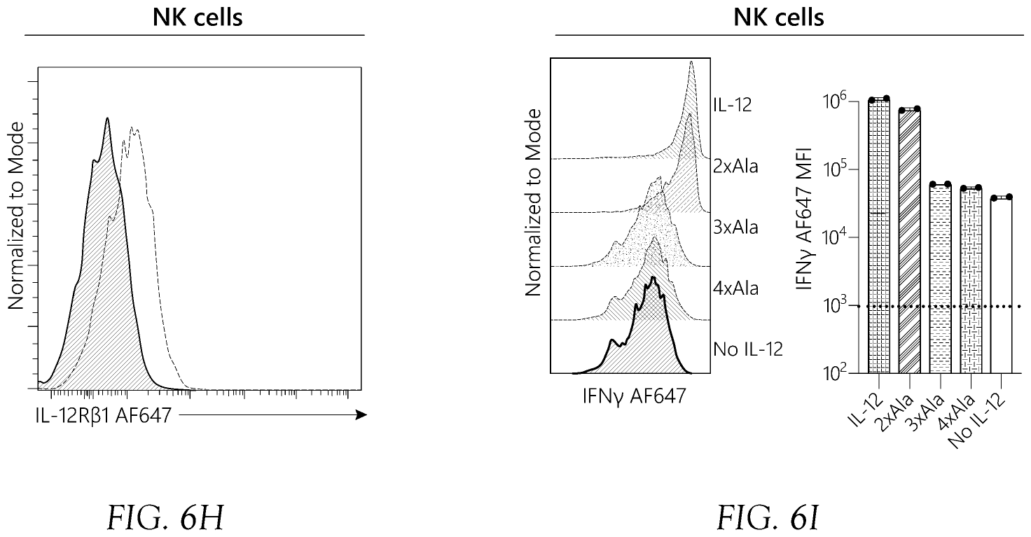

To assess the ability of IL-12 variants to stimulate IFNγ production in NK cells, additional experiments were performed to stimulate cells with IL-12 variants in the presence of IL-18 for 48 hours prior to analysis of IFNγ induction by intracellular cytokine stain. IL-12 and IL-18 stimulation induced robust IFNγ expression, a response that was attenuated in the (2×Ala) mutants and abrogated in the 3×Ala and 4×Ala variants as measured by intracellular cytokine stain and supernatant ELISA (see, e.g., FIG. 4B). Thus, while IL-12 induces robust IFNγ expression in both CD8+ T cells and NK cells, the (3×Ala) and (4×Ala) partial agonists preferentially support IFNγ induction in antigen experienced CD8+ T cells with reduced activity on NK cells (FIGS. 4C and 6A). These results suggest that activated CD8+ T cells are more tolerant to mutations in IL-12p40 due to increased IL-12Rβ1 surface expression and that this may represent a novel mechanism by which to alter the cell-type specificity of IL-12 signaling in order to reduce NK cell mediated toxicity. Unlike T cells, which require stimulation through the TCR to respond to IL-12, NK cells produce IFNγ in response to IL-12 in combination with the IL-1 family cytokine IL-18 (FIG. 6B). IL-12 and IL-18 stimulation induced robust IFNγ expression, a response that was attenuated with 3×Ala and 4×Ala variants as measured by intracellular cytokine stain (FIGS. 4B, 6C, and 6D) and supernatant ELISA (FIG. 6E). These results were confirmed and extended with a larger panel of IL-12 partial agonists (FIGS. 4D-4G).

IL-12 and IL-18 also promoted upregulation of ling at the transcript level following 8-h stimulation, an effect that was reduced with 3×Ala/IL-18 stimulation (FIG. 6F); however, under these conditions, induction of Tigit by IL-12 was not observed (FIG. 6G). Previously, the γc family cytokines IL-2 and IL-15 have been shown to modulate the activity of NK cells and lead to upregulation of IL-12 receptor components. Consistent with these reports, additional experiments were performed to demonstrate that pre-activation of NK cells with IL-2 led to a slight upregulation of IL-12Rβ1 (FIG. 6H). Addition of IL-2 to NK cell cultures increased IFNγ production above IL-18 alone; however, it was observed that IL-2 did not synergize with 3×Ala and 4×Ala to enhance IFNγ induction above IL-2/IL-18 (FIG. 6I).

Additional experiments were performed to assess IL-12Rβ1 expression and IFNγ production in human peripheral blood mononuclear cells (PBMCs), which helped determine if human IL-12 partial agonists were capable of eliciting cell-type-specific responses. As summarized in FIGS. 7A-7D, it was observed that similar to the findings in mouse, TCR stimulation enhanced IL-12Rβ1 expression in CD8⁺ T cells above that of nonactivated T cells and NK cells (FIGS. 7A and 7B). In these experiments, analogous IL-12 muteins were generated and tested for pSTAT4 signaling in CD8⁺ T cell blasts (FIGS. 7C-7D and 7E). It was observed that human IL-12 partial agonists preferentially supported induction of IFNγ by CD8⁺ T cells relative to NK cells (FIGS. 7C-7D, 7F-7G). These findings indicate that upregulation of IL-12Rβ1 is a conserved mechanism used by T cells to enhance sensitivity to IL-12 signaling and that IL-12 partial agonists are capable of biasing signaling toward T cells in both human and mouse.

### Example 5

### IL-12 Partial Agonists Promote Antigen-Specific Tumor Killing

This Example describes the results from experiments performed to demonstrate that IL-12 partial agonists promote antigen-specific tumor killing.

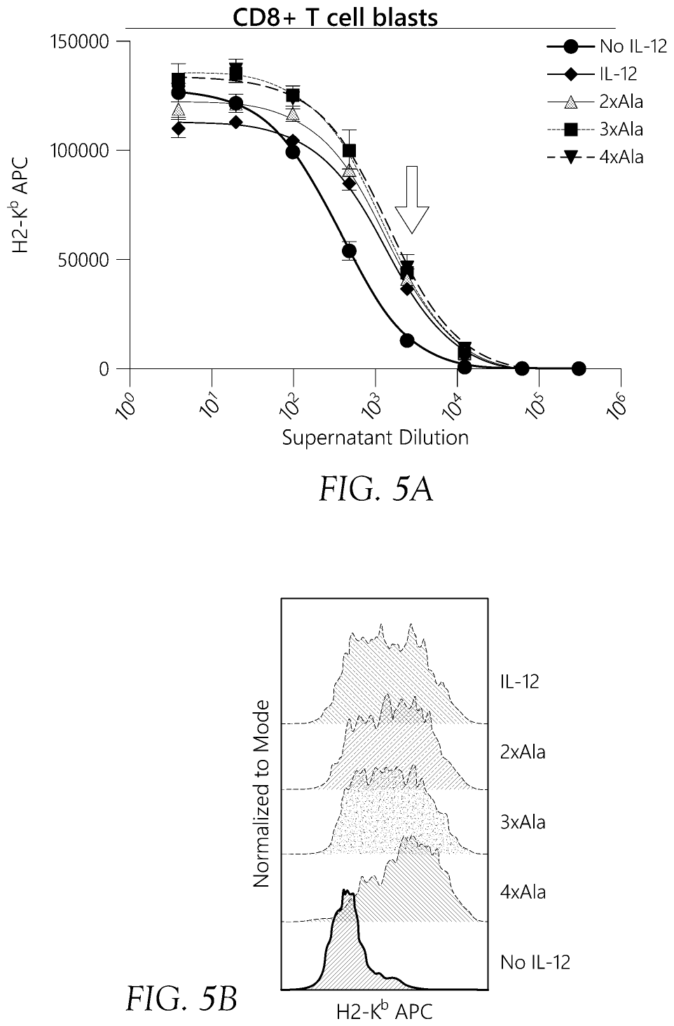

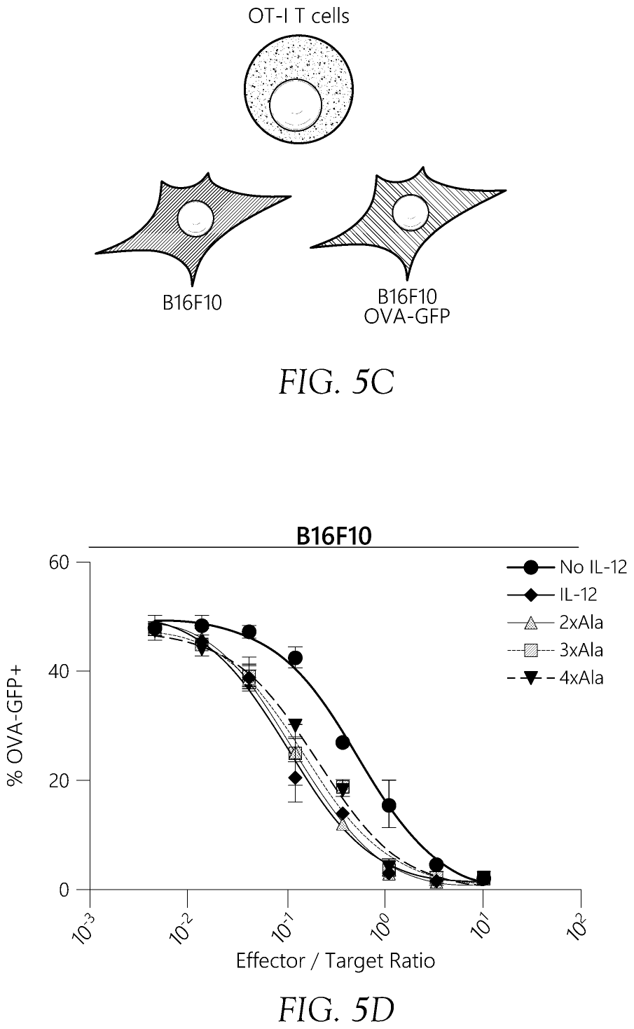

In CD8+ T cells, IL-12 acts to potentiate antigen-specific killing of tumors and virally infected cells (Schurich et al., 2013). The effects of IL-12 are mediated by upregulation of cytotoxic factors, such as granzyme B, and secretion of inflammatory cytokines including IFNγ (Aste-Amezaga et al., 1994). A well described role of IFNγ in tumor cell killing is the upregulation of MHC-I, which can render transformed cells sensitive to T cell surveillance (Zhou, 2009). To determine if IL-12 induced IFNγ leads to upregulation of MHC-I on tumor cell lines, supernatants from OT-I effectors generated with or without IL-12 partial agonists were harvested and then added to the B16F10 murine melanoma cell line and assessed MHC-I surface expression by antibody stain following overnight incubation. Consistent with elevated levels of IFNγ measured by intracellular cytokine stain, supernatants from IL-12 and partial agonist cultures more potently induced MHC-I expression than supernatant generated in the absence of IL-12 (FIG. 5A).

The finding described herein that IL-12 partial agonists promote IFNγ production and subsequent upregulation of MHC-I on tumor cell lines led to further examination of the capacity of IL-12 partial agonists to potentiate tumor cell killing. To measure antigen-specific CD8+ T cell killing, B16F10 cells were transduced with a plasmid containing ovalbumin along with a GFP marker (OVA-GFP) and mixed them with wild-type B16F10 cells. This mixture was incubated with OT-I effectors and the frequency of OVA-GFP expressing cells was used to measure antigen-specific tumor cell killing (FIG. 5B). OT-I effectors generated in the presence of IL-12 or partial agonists were able to kill OVA expressing tumor cells at a lower effector cell to target cell ratio, indicating increased potency of antitumor response (FIG. 5C). Together, these data indicate that IL-12 partial agonists with reduced affinity for IL-12Rβ1 promote IFNγ production and tumor cell killing by antigen-specific CD8+ T cells with reduced activity on NK cells.

### Example 6

### IL-12 Partial Agonists Support Antigen-Specific T Cell Response with Reduced NK Cell Activation In Vivo

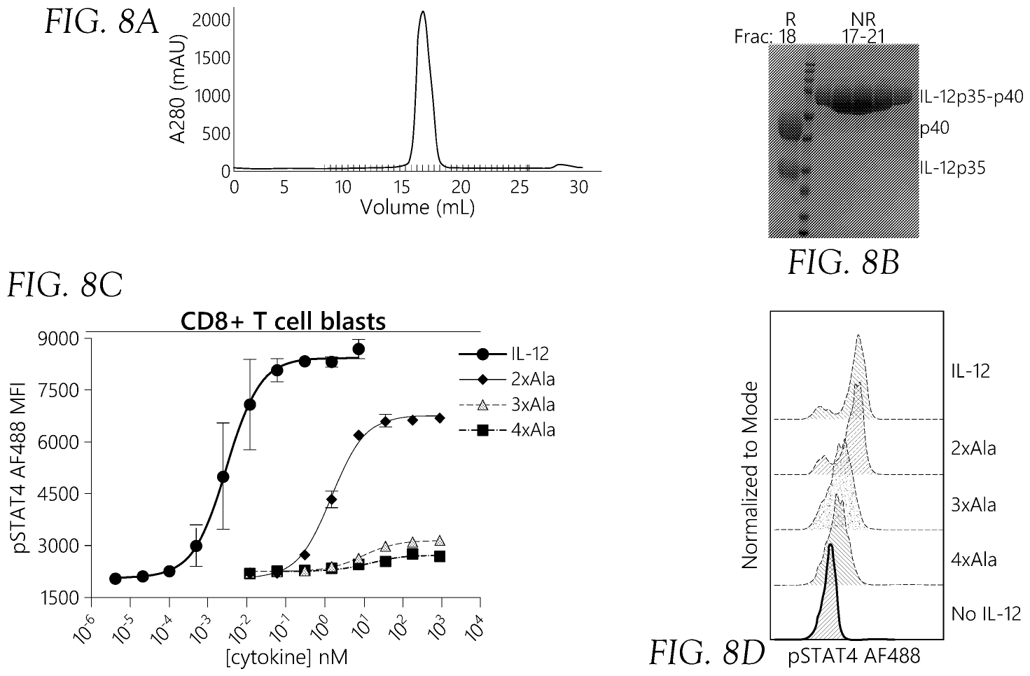

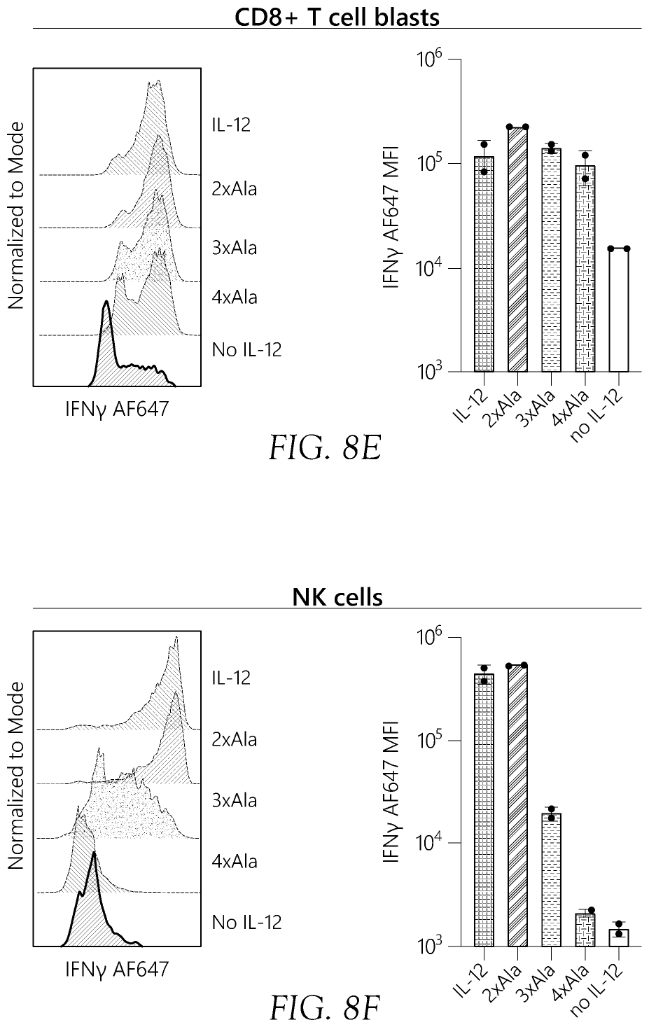

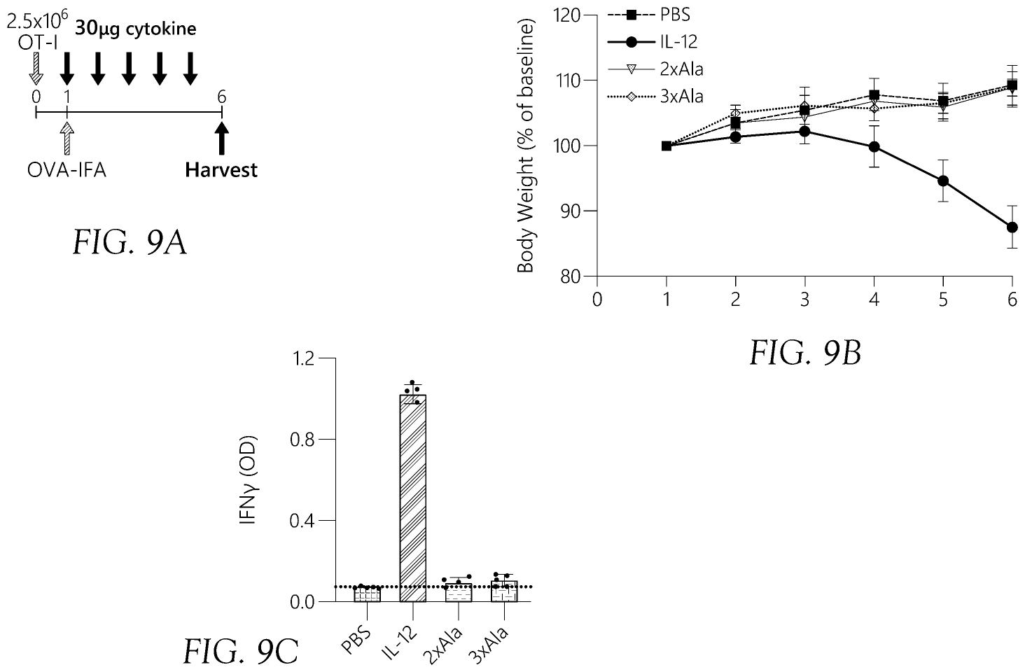

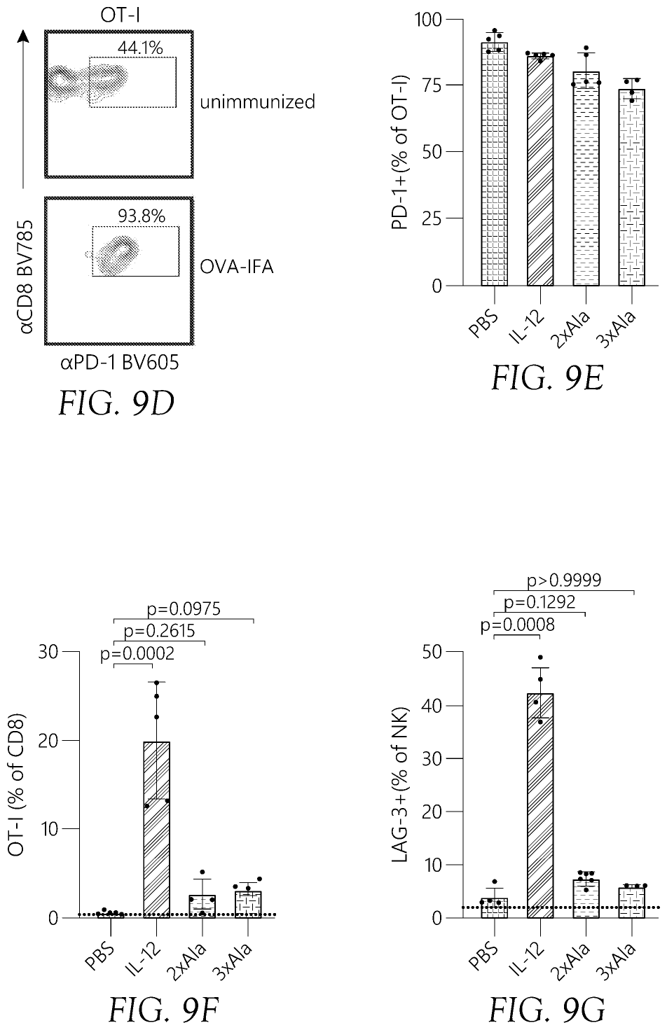

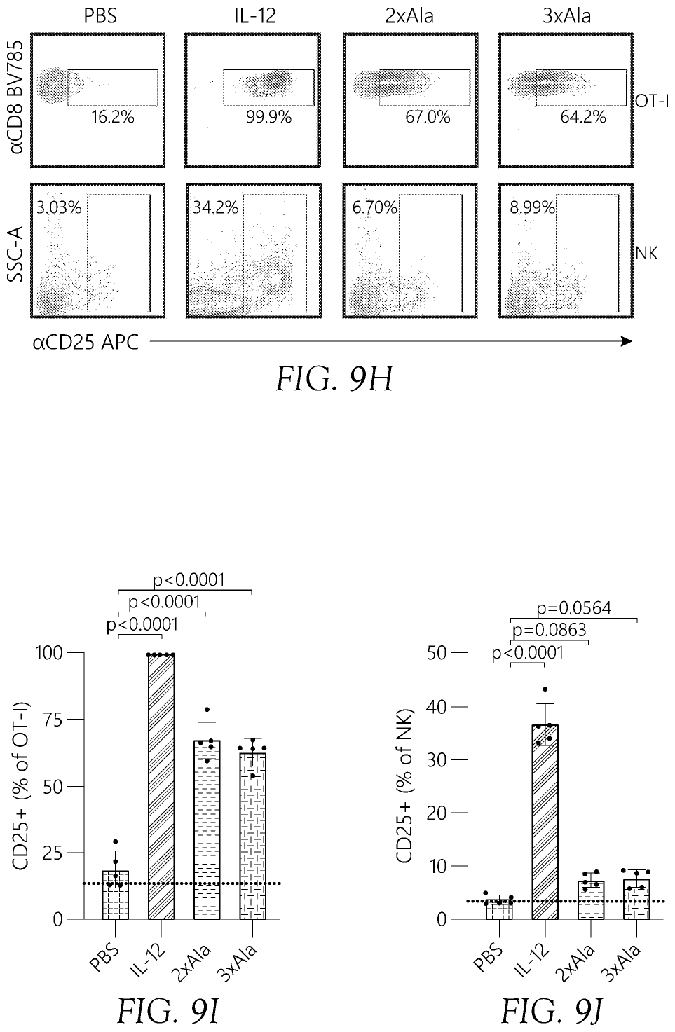

To test whether IL-12 partial agonists elicit cell-type specific responses in vivo, OT-I CD8+ T cells were adoptively transferred into Thy1.1 congenic recipients and immunized with OVA (257-264) in Incomplete Freud's Adjuvant (OVA-IFA) followed by daily cytokine administration for 5 days (FIG. 9A). For in vivo studies, IL-12 and partial agonists were expressed in mammalian cells (Expi293F). It was then confirmed that mammalian-expressed IL-12 partial agonists retain cell-type bias in vitro, as seen for the baculovirus-expressed material used previously (FIGS. 8A-8E).

It was observed that treatment with IL-12 but not 2×Ala and 3×Ala induced weight loss and elevated levels of IFNg in serum (FIGS. 9B-9C). To assess the impact of immunization on T cell activation, expression of the inhibitory receptor, PD-1, on OT-I T cells was monitored. Immunization increased the frequency of PD-1+ OT-I T cells independent of cytokine treatment, indicating activation of adoptively transferred cells (FIGS. 9D-9E). The effects of immunization were potentiated by IL-12 which increased the frequency of OT-I T cells in the draining lymph node, an effect not seen with partial agonists (FIG. 9F). Within NK cells, IL-12 but not partial agonists increased a population of activated NK cells as measured by expression of the inhibitory receptor, LAG-3 (FIG. 9G).

Previously the IL-2Rα chain, CD25, has been described as a marker of activated T cells and NK cells. IL-12 strongly upregulated CD25 expression on both OT-I T cells and NK cells while the 2×Ala and 3×Ala partial agonists led to intermediate upregulation of CD25 on OT-I T cells without increasing expression on NK cells (FIGS. 9H-9J). Interestingly, it was observed that while the 2×Ala variant did not exhibit as significant T/NK cell bias as the 3×Ala variant in vitro (FIGS. 8E-8F), it shows comparably strong T/NK cell bias to 3×Ala in vivo, highlighting that the therapeutic window will likely be quantitatively different in vitro versus in vivo. These results indicate that IL-12 partial agonists support intermediate levels of T cell activation with reduced NK cell stimulation and toxicity in vivo.

### Example 7

### IL-12 Partial Agonists Support Anti-Tumor Immunity with Reduced Toxicity Relative to IL-12

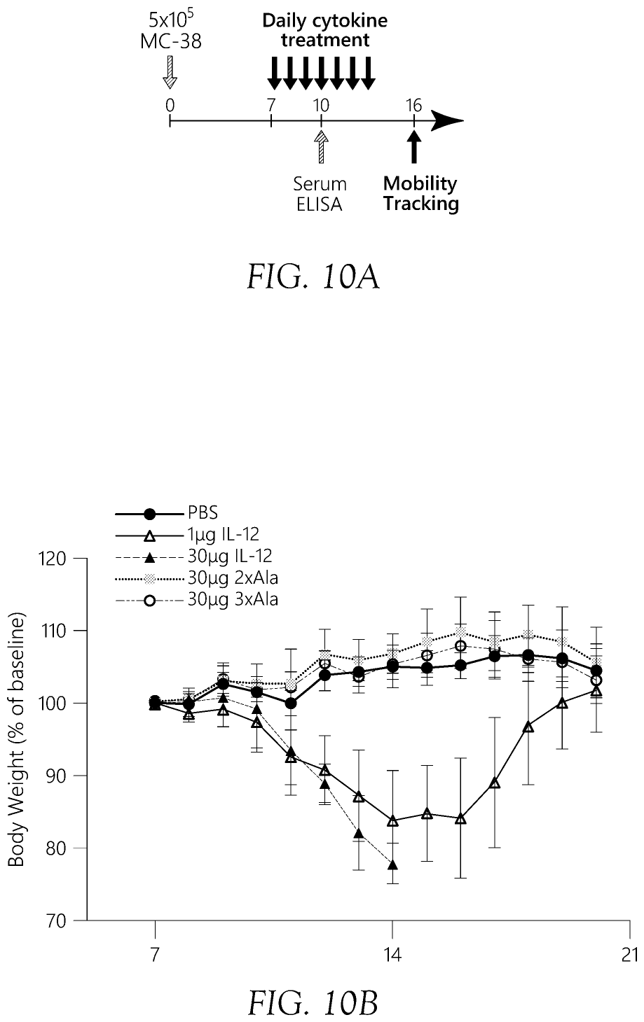

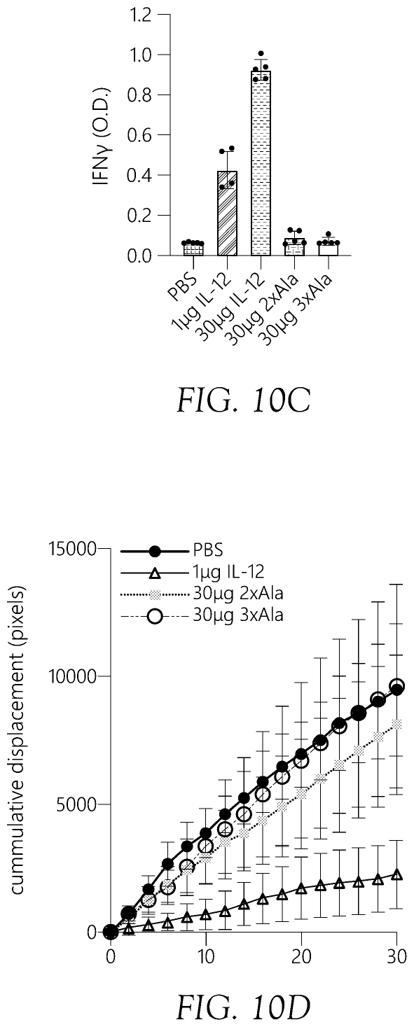

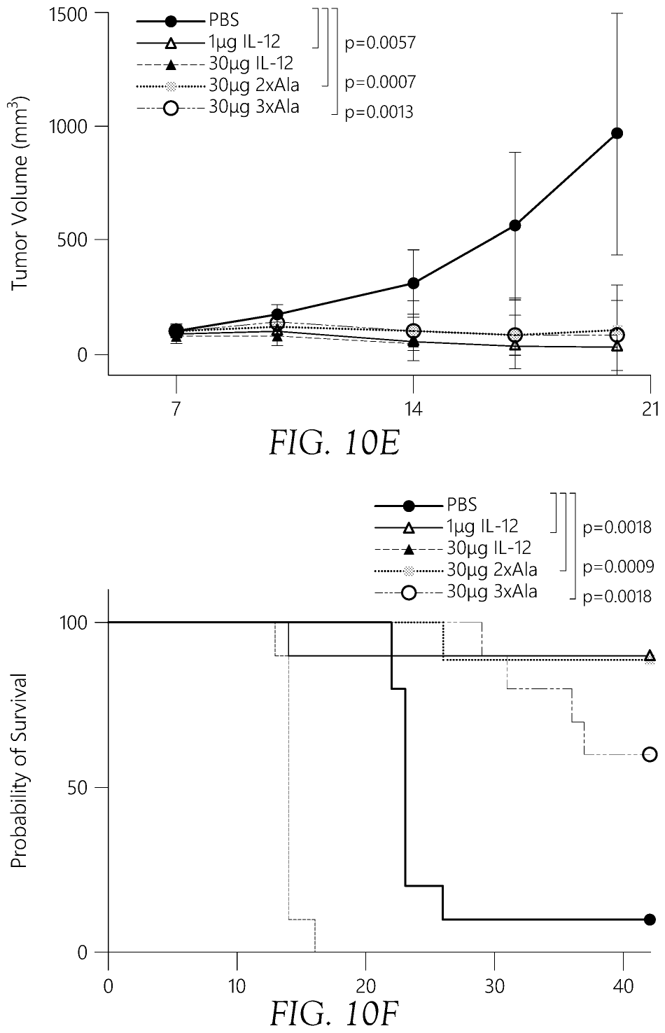

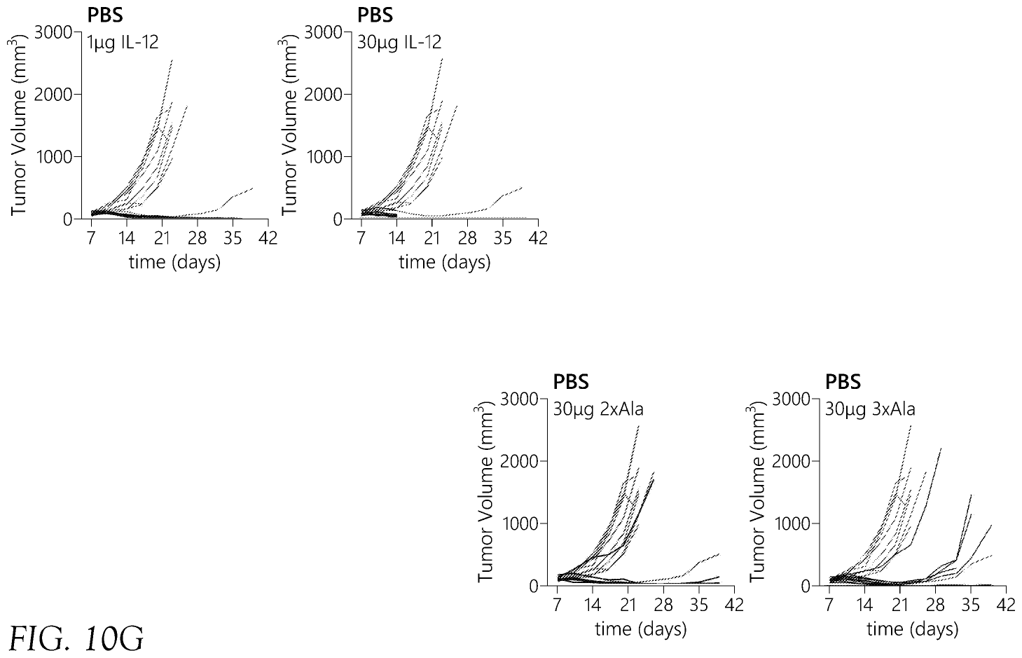

Based on in vitro characterization and in vivo cell profiling, it was concluded that IL-12 partial agonists could be capable of supporting anti-tumor T cell immunity without systemic toxicity by biasing the activity of IL-12 towards antigen-specific T cells and away from NK cells. To determine the ability of IL-12 partial agonists to provide therapeutic benefit in vivo, additional experiments were performed on tumor using the colon adenocarcinoma MC-38, which has been shown to be responsive to IL-12. In these experiments, mice were engrafted with MC-38 for 1 week prior to initiation of daily cytokine treatment for 7 days (FIG. 10A). Daily IL-12 administration, either at 1 μg or 30 μg, resulted in profound toxicity as measured by weight loss (FIG. 10B), elevated serum IFNγ (FIG. 10C) and reduced mobility (FIG. 10D). It was observed that all mice administered 30 μg of IL-12 succumbed to lethal toxicity between days 13 and 15. As a result, the mobility of these mice on day 16 was not performed. In contrast, the 2×Ala and 3×Ala partial agonists were well tolerated and did not induce toxicity in tumor-bearing mice.

It was further observed that both IL-12 and partial agonists attenuated tumor growth and prolonged survival relative to treatment with PBS (FIGS. 10E-10H). However, the 2×Ala and 3×Ala partial agonists did so without inducing systemic toxicity observed with IL-12 administration. These results provide additional in vivo support for the hypothesis that the biased agonists, designed based on the structure of the IL-12Rβ1 shared interface, have the capacity to decouple T cell from NK cell activation, significantly reducing IL-12 pleiotropy.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

Anderson, R., Macdonald, I., Corbett, T., Hacking, G., Lowdell, M. W., and Prentice, H. G. (1997). Construction and biological characterization of an interleukin-12 fusion protein (Flexi-12): delivery to acute myeloid leukemic blasts using adeno-associated virus. Hum Gene Ther 8, 1125-1135.
Ardolino, M., Azimi, C. S., Iannello, A., Trevino, T. N., Horan, L., Zhang, L., Deng, W., Ring, A. M., Fischer, S., Garcia, K. C., et al. (2014). Cytokine therapy reverses NK cell anergy in MHC-deficient tumors. J Clin Invest 124, 4781-4794.
Aste-Amezaga, M., D'Andrea, A., Kubin, M., and Trinchieri, G. (1994). Cooperation of natural killer cell stimulatory factor/interleukin-12 with other stimuli in the induction of cytokines and cytotoxic cell-associated molecules in human T and NK cells. Cell Immunol 156, 480-492.
Bekaii-Saab, T. S., Roda, J. M., Guenterberg, K. D., Ramaswamy, B., Young, D. C., Ferketich, A. K., Lamb, T. A., Greyer, M. R., Shapiro, C. L., and Carson, W. E., 3rd (2009). A phase I trial of paclitaxel and trastuzumab in combination with interleukin-12 in patients with HER2/neu-expressing malignancies. Mol Cancer Ther 8, 2983-2991.
Benson, J. M., Sachs, C. W., Treacy, G., Zhou, H., Pendley, C. E., Brodmerkel, C. M., Shankar, G., and Mascelli, M. A. (2011). Therapeutic targeting of the IL-12/23 pathways: generation and characterization of ustekinumab. Nat Biotechnol 29, 615-624.
Best, M., (2009). Biochemistry 48(28): 6571-6584.
Bloch, Y., Bouchareychas, L., Merceron, R., Skladanowska, K., Van den Bossche, L., Detry, S., Govindarajan, S., Elewaut, D., Haerynck, F., Dullaers, M., etal. (2018). Structural Activation of Pro-inflammatory Human Cytokine IL-23 by Cognate IL-23 Receptor Enables Recruitment of the Shared Receptor IL-12Rbeta1. Immunity 48, 45-58 e46.
Boulanger, M. J., Bankovich, A. J., Kortemme, T., Baker, D., and Garcia, K. C. (2003a). Convergent mechanisms for recognition of divergent cytokines by the shared signaling receptor gp130. Mol Cell 12, 577-589.
Boulanger, M. J., Chow, D. C., Brevnova, E. E., and Garcia, K. C. (2003b). Hexameric structure and assembly of the interleukin-6/IL-6 alpha-receptor/gp130 complex. Science 300, 2101-2104.
Brunda, M. J., Luistro, L., Warrier, R. R., Wright, R. B., Hubbard, B. R., Murphy, M., Wolf, S. F., and Gately, M. K. (1993). Antitumor and antimetastatic activity of interleukin 12 against murine tumors. J Exp Med 178, 1223-1230.
Chao, G., Lau, W. L., Hackel, B. J., Sazinsky, S. L., Lippow, S. M., and Wittrup, K. D. (2006). Isolating and engineering human antibodies using yeast surface display. Nat Protoc 1, 755-768.
Chua, A. O., Chizzonite, R., Desai, B. B., Truitt, T. P., Nunes, P., Minetti, L. J., Warrier, R. R., Presky, D. H., Levine, J. F., Gately, M. K., etal. (1994). Expression cloning of a human IL-12 receptor component. A new member of the cytokine receptor superfamily with strong homology to gp130. J Immunol 153, 128-136.
Collison, L. W., Workman, C. J., Kuo, T. T., Boyd, K., Wang, Y., Vignali, K. M., Cross, R., Sehy, D., Blumberg, R. S., and Vignali, D. A. (2007). The inhibitory cytokine IL-35 contributes to regulatory T-cell function. Nature 450, 566-569.
Cooper, A. M., Kipnis, A., Turner, J., Magram, J., Ferrante, J., and Orme, I. M. (2002). Mice lacking bioactive IL-12 can generate protective, antigen-specific cellular responses to mycobacterial infection only if the IL-12 p40 subunit is present. J Immunol 168, 1322-1327.
Decken, K., Kohler, G., Palmer-Lehmann, K., Wunderlin, A., Mattner, F., Magram, J., Gately, M. K., and Alber, G. (1998). Interleukin-12 is essential for a protective Th1 response in mice infected with Cryptococcus neoformans. Infect Immun 66, 4994-5000.
DeLano, W. L., Ultsch, M. H., de Vos, A. M., and Wells, J. A. (2000). Convergent solutions to binding at a protein-protein interface. Science 287, 1279-1283.
Desmyter, A., Spinelli, S., Boutton, C., Saunders, M., Blachetot, C., de Haard, H., Denecker, G., Van Roy, M., Cambillau, C., and Rommelaere, H. (2017). Neutralization of Human Interleukin 23 by Multivalent Nanobodies Explained by the Structure of Cytokine-Nanobody Complex. Front Immunol 8, 884.
Dieters, et al., (2004). Site-specific PEGylation of proteins containing unnatural amino acids. Bioorganic and Medicinal Chemistry Letters 14(23):5743-5745.
Disis, M. L., Watt, W. C., and Cecil, D. L. (2014). Th1 epitope selection for clinically effective cancer vaccines. Oncoimmunology 3, e954971.
Dozier and Distefano. Site-Specific PEGylation of Therapeutic Proteins. International Journal of Molecular Science 16(10): 25831-25864, 2015.
Emsley, P., Lohkamp, B., Scott, W. G., and Cowtan, K. (2010). Features and development of Coot. Acta Crystallogr D Biol Crystallogr 66, 486-501.
Fewell, J. G., Matar, M. M., Rice, J. S., Brunhoeber, E., Slobodkin, G., Pence, C., Worker, M., Lewis, D. H., and Anwer, K. (2009). Treatment of disseminated ovarian cancer using nonviral interleukin-12 gene therapy delivered intraperitoneally. J Gene Med 11, 718-728.
Hogquist, K. A., Jameson, S. C., Heath, W. R., Howard, J. L., Bevan, M. J., and Carbone, F. R. (1994). T cell receptor antagonist peptides induce positive selection. Cell 76, 17-27.
Holscher, C., Atkinson, R. A., Arendse, B., Brown, N., Myburgh, E., Alber, G., and Brombacher, F. (2001). A protective and agonistic function of IL-12p40 in mycobacterial infection. J Immunol 167, 6957-6966.
Huyton, T., Zhang, J. G., Luo, C. S., Lou, M. Z., Hilton, D. J., Nicola, N. A., and Garrett, T. P. (2007). An unusual cytokine:Ig-domain interaction revealed in the crystal structure of leukemia inhibitory factor (LIF) in complex with the LIF receptor. Proc Natl Acad Sci USA 104, 12737-12742.
Kabsch, W. (2010). Xds. Acta Crystallogr D Biol Crystallogr 66, 125-132.
Kobayashi, M., Fitz, L., Ryan, M., Hewick, R. M., Clark, S. C., Chan, S., Loudon, R., Sherman, F., Perussia, B., and Trinchieri, G. (1989). Identification and purification of natural killer cell stimulatory factor (NKSF), a cytokine with multiple biologic effects on human lymphocytes. J Exp Med 170, 827-845.
Kopp, T., Riedl, E., Bangert, C., Bowman, E. P., Greisenegger, E., Horowitz, A., Kittler, H., Blumenschein, W. M., McClanahan, T. K., Marbury, T., et al. (2015). Clinical improvement in psoriasis with specific targeting of interleukin-23. Nature 521, 222-226.
Koutruba, N., Emer, J., and Lebwohl, M. (2010). Review of ustekinumab, an interleukin-12 and interleukin-23 inhibitor used for the treatment of plaque psoriasis. Ther Clin Risk Manag 6, 123-141.
Lasek, W., Zagozdzon, R., and Jakobisiak, M. (2014). Interleukin 12: still a promising candidate for tumor immunotherapy? Cancer Immunol Immunother 63, 419-435.
Leahy, D. J., Hendrickson, W. A., Aukhil, I., and Erickson, H. P. (1992). Structure of a fibronectin type III domain from tenascin phased by MAD analysis of the selenomethionyl protein. Science 258, 987-991.
Liang, D., Zuo, A., Shao, H., Born, W. K., O'Brien, R. L., Kaplan, H. J., and Sun, D. (2013). IL-23 receptor expression on gammadelta T cells correlates with their enhancing or suppressive effects on autoreactive T cells in experimental autoimmune uveitis. J Immunol 191, 1118-1125.
Liebschner, D., Afonine, P. V., Baker, M. L., Bunkoczi, G., Chen, V. B., Croll, T. I., Hintze, B., Hung, L. W., Jain, S., McCoy, A. J., et al. (2019). Macromolecular structure determination using X-rays, neutrons and electrons: recent developments in Phenix. Acta Crystallogr D Struct Biol 75, 861-877.
Littman, D. R., and Rudensky, A. Y. (2010). Th17 and regulatory T cells in mediating and restraining inflammation. Cell 140, 845-858.
Lothar Steidler, Sabine Neirynck, Nathalie Huyghebaert,Veerle Snoeck, An Vermeire, Bruno Goddeeris, Eric Cox, Jean Paul Remon and Erik Remaut. (2003). Biological containment of genetically modified Lactococcus lactis for intestinal delivery of human interleukin 10. Nature Biotechnology, Vol. 21, No. 7, July 2003.
Luo, J., Wu, S. J., Lacy, E. R., Orlovsky, Y., Baker, A., Teplyakov, A., Obmolova, G., Heavner, G. A., Richter, H. T., and Benson, J. (2010). Structural basis for the dual recognition of IL-12 and IL-23 by ustekinumab. J Mol Biol 402, 797-812.
Lupardus, P. J., and Garcia, K. C. (2008). The structure of interleukin-23 reveals the molecular basis of p40 subunit sharing with interleukin-12. J Mol Biol 382, 931-941.
Mattner, F., Magram, J., Ferrante, J., Launois, P., Di Padova, K., Behin, R., Gately, M. K., Louis, J. A., and Alber, G. (1996). Genetically resistant mice lacking interleukin-12 are susceptible to infection with Leishmania major and mount a polarized Th2 cell response. Eur J Immunol 26, 1553-1559.
Morin, A., Eisenbraun, B., Key, J., Sanschagrin, P. C., Timony, M. A., Ottaviano, M., and Sliz, P. (2013). Collaboration gets the most out of software. Elife 2, e01456.
Murray, P. J. (2007). The JAK-STAT signaling pathway: input and output integration. J Immunol 178, 2623-2629.
Oh Jee-Hwan, Schueler L. Kathryn, Donnie S. Stapleton, Laura M. Alexander, Chi-Liang Eric Yen, Mark P. Keller, Alan D. Attie, Jan-Peter van Pijkeren. (2020). Secretion of Recombinant Interleukin-22 by Engineered Lactobacillus reuteri Reduces Fatty Liver Disease in a Mouse Model of Diet-Induced Obesity. mSphere, Vol. 5, Issue 3, May/June 2020.
Oppmann, B., Lesley, R., Blom, B., Timans, J. C., Xu, Y., Hunte, B., Vega, F., Yu, N., Wang, J., Singh, K., et al. (2000). Novel p19 protein engages IL-12p40 to form a cytokine, IL-23, with biological activities similar as well as distinct from IL-12. Immunity 13, 715-725.
Parham, C., Chirica, M., Timans, J., Vaisberg, E., Travis, M., Cheung, J., Pflanz, S., Zhang, R., Singh, K. P., Vega, F., et al. (2002). A receptor for the heterodimeric cytokine IL-23 is composed of IL-12Rbeta1 and a novel cytokine receptor subunit, IL-23R. J Immunol 168, 5699-5708.
Pasche, N., Wulhfard, S., Pretto, F., Carugati, E., and Neri, D. (2012). The antibody-based delivery of interleukin-12 to the tumor neovasculature eradicates murine models of cancer in combination with paclitaxel. Clin Cancer Res 18, 4092-4103.
Pegram, H. J., Lee, J. C., Hayman, E. G., Imperato, G. H., Tedder, T. F., Sadelain, M., and Brentjens, R. J. (2012). Tumor-targeted T cells modified to secrete IL-12 eradicate systemic tumors without need for prior conditioning. Blood 119, 4133-4141.
Pflanz, S., Timans, J. C., Cheung, J., Rosales, R., Kanzler, H., Gilbert, J., Hibbert, L., Churakova, T., Travis, M., Vaisberg, E., et al. (2002). IL-27, a heterodimeric cytokine composed of EBI3 and p28 protein, induces proliferation of naive CD4+ T cells. Immunity 16, 779-790.
Presky, D. H., Yang, H., Minetti, L. J., Chua, A. O., Nabavi, N., Wu, C. Y., Gately, M. K., and Gubler, U. (1996). A functional interleukin 12 receptor complex is composed of two beta-type cytokine receptor subunits. Proc Natl Acad Sci USA 93, 14002-14007.
Riethmueller, S., Somasundaram, P., Ehlers, J. C., Hung, C. W., Flynn, C. M., Lokau, J., Agthe, M., Dusterhoft, S., Zhu, Y., Grotzinger, J., et al. (2017). Proteolytic Origin of the Soluble Human IL-6R In Vivo and a Decisive Role of N-Glycosylation. PLoS Biol 15, e2000080.
Rossjohn, J., Gras, S., Miles, J. J., Turner, S. J., Godfrey, D. I., and McCluskey, J. (2015). T cell antigen receptor recognition of antigen-presenting molecules. Annu Rev Immunol 33, 169-200.
Schroder, J., Moll, J. M., Baran, P., Grotzinger, J., Scheller, J., and Floss, D. M. (2015). Non-canonical interleukin 23 receptor complex assembly: p40 protein recruits interleukin 12 receptor beta1 via site II and induces p19/interleukin 23 receptor interaction via site III. J Biol Chem 290, 359-370.
Schurich, A., Pallett, L. J., Lubowiecki, M., Singh, H. D., Gill, U. S., Kennedy, P. T., Nastouli, E., Tanwar, S., Rosenberg, W., and Maini, M. K. (2013). The third signal cytokine IL-12 rescues the anti-viral function of exhausted HBV-specific CD8 T cells. PLoS Pathog 9, e1003208.
Skiniotis, G., Lupardus, P. J., Martick, M., Walz, T., and Garcia, K. C. (2008). Structural organization of a full-length gp130/LIF-R cytokine receptor transmembrane complex. Mol Cell 31, 737-748.
Szabo, S. J., Dighe, A. S., Gubler, U., and Murphy, K. M. (1997). Regulation of the interleukin (IL)-12R beta 2 subunit expression in developing T helper 1 (Th1) and Th2 cells. J Exp Med 185, 817-824.
Tait Wojno, E. D., Hunter, C. A., and Stumhofer, J. S. (2019). The Immunobiology of the Interleukin-12 Family: Room for Discovery. Immunity 50, 851-870.
Takeshita, T., Asao, H., Ohtani, K., Ishii, N., Kumaki, S., Tanaka, N., Munakata, H., Nakamura, M., and Sugamura, K. (1992). Cloning of the gamma chain of the human IL-2 receptor. Science 257, 379-382.
Tamura, T., Nishi, T., Goto, T., Takeshima, H., Dev, S. B., Ushio, Y., and Sakata, T. (2001). Intratumoral delivery of interleukin 12 expression plasmids with in vivo electroporation is effective for colon and renal cancer. Hum Gene Ther 12, 1265-1276.
Thul, P. J., Akesson, L., Wiking, M., Mandessian, D., Geladaki, A., Ait Blal, H., Alm, T., Asplund, A., Bjork, L., Breckels, L. M., et al. (2017). A subcellular map of the human proteome. Science 356.
Trinchieri, G. (2003). Interleukin-12 and the regulation of innate resistance and adaptive immunity. Nat Rev Immunol 3, 133-146.
Tseng, D., Volkmer, J. P., Willingham, S. B., Contreras-Trujillo, H., Fathman, J. W., Fernhoff, N. B., Seita, J., Inlay, M. A., Weiskopf, K., Miyanishi, M., et al. (2013). Anti-CD47 antibody-mediated phagocytosis of cancer by macrophages primes an effective antitumor T-cell response. Proc Natl Acad Sci USA 110, 11103-11108.
Vance, R. E., Eichberg, M. J., Portnoy, D. A., and Raulet, D. H. (2017). Listening to each other: Infectious disease and cancer immunology. Sci Immunol 2.
Vignali, D. A., and Kuchroo, V. K. (2012). IL-12 family cytokines: immunological playmakers. Nat Immunol 13, 722-728.
Villarino, A. V., Kanno, Y., Ferdinand, J. R., and O'Shea, J. J. (2015). Mechanisms of Jak/STAT signaling in immunity and disease. J Immunol 194, 21-27.
Vonrhein, C., Blanc, E., Roversi, P., and Bricogne, G. (2007). Automated structure solution with autoSHARP. Methods Mol Biol 364, 215-230.
Wang, X., Lupardus, P., Laporte, S. L., and Garcia, K. C. (2009). Structural biology of shared cytokine receptors. Annu Rev Immunol 27, 29-60.
Wang, X., Wei, Y., Xiao, H., Liu, X., Zhang, Y., Han, G., Chen, G., Hou, C., Ma, N., Shen, B., et al. (2016). A novel IL-23p19/Ebi3 (IL-39) cytokine mediates inflammation in Lupus-like mice. Eur J Immunol 46, 1343-1350.
Watford, W. T., Hissong, B. D., Bream, J. H., Kanno, Y., Muul, L., and O'Shea, J. J. (2004). Signaling by IL-12 and IL-23 and the immunoregulatory roles of STAT4. Immunol Rev 202, 139-156.
Yen, D., Cheung, J., Scheerens, H., Poulet, F., McClanahan, T., McKenzie, B., Kleinschek, M. A., Owyang, A., Mattson, J., Blumenschein, W., et al. (2006). IL-23 is essential for T cell-mediated colitis and promotes inflammation via IL-17 and IL-6. J Clin Invest 116, 1310-1316.
Yeste, A., Mascanfroni, I. D., Nadeau, M., Burns, E. J., Tukpah, A. M., Santiago, A., Wu, C., Patel, B., Kumar, D., and Quintana, F. J. (2014). IL-21 induces IL-22 production in CD4+ T cells. Nat Commun 5, 3753.
Yodoi, J., Teshigawara, K., Nikaido, T., Fukui, K., Noma, T., Honjo, T., Takigawa, M., Sasaki, M., Minato, N., Tsudo, M., et al. (1985). TCGF (IL 2)-receptor inducing factor(s). I. Regulation of IL 2 receptor on a natural killer-like cell line (YT cells). J Immunol 134, 1623-1630.
Yoon, C., Johnston, S. C., Tang, J., Stahl, M., Tobin, J. F., and Somers, W. S. (2000). Charged residues dominate a unique interlocking topography in the heterodimeric cytokine interleukin-12. EMBO J 19, 3530-3541.
Yoshimoto, T., Takeda, K., Tanaka, T., Ohkusu, K., Kashiwamura, S., Okamura, H., Akira, S., and Nakanishi, K. (1998). IL-12 up-regulates IL-18 receptor expression on T cells, Th1 cells, and B cells: synergism with IL-18 for IFN-gamma production. J Immunol 161, 3400-3407.
Zhou, F. (2009). Molecular mechanisms of IFN-gamma to up-regulate MHC class I antigen processing and presentation. Int Rev Immunol 28, 239-260.
Zhou, L., Ivanov, I I, Spolski, R., Min, R., Shenderov, K., Egawa, T., Levy, D. E., Leonard, W. J., and Littman, D. R. (2007). IL-6 programs T(H)-17 cell differentiation by promoting sequential engagement of the IL-21 and IL-23 pathways. Nat Immunol 8, 967-974.
Zhu, J., Yamane, H., and Paul, W. E. (2010). Differentiation of effector CD4 T cell populations (*). Annu Rev Immunol 28, 445-489.
Zitvogel, L., Tahara, H., Robbins, P. D., Storkus, W. J., Clarke, M. R., Nalesnik, M. A., and Lotze, M. T. (1995). Cancer immunotherapy of established tumors with IL-12. Effective delivery by genetically engineered fibroblasts. J Immunol 155, 1393-1403.

What is claimed is:

1. A recombinant polypeptide comprising:
an amino acid sequence having one or more 70%, 80%, 90%, 95%, 99%, or 100% sequence identity to an interleukin 12 subunit p40 (IL-12p40) polypeptide having the amino acid sequence of SEQ ID NO: 1;
and further comprising one or more amino acid substitution at a position corresponding to an amino acid residue selected from the group consisting of X37, X39, X40, X41, X80, X81, X82, X106, X108, X115, X216, X217, X218, and X219 of SEQ ID NO: 1.

2. The recombinant polypeptide of claim 1, wherein the one or more amino acid substitution is at a position corresponding to an amino acid residue selected from the group consisting of X37, X39, X40, X81, X82, X106, X217, and X219 of SEQ ID NO: 1.

3. The recombinant polypeptide of any one of claims 1 to 2, wherein the one or more amino acid substitution is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution.

4. The recombinant polypeptide of any one of claims 1 to 3, wherein the one or more amino acid substitution is at a position corresponding to an amino acid residue selected from the group consisting of W37, P39, D40, A41, K80, E81, F82, K106, E108, D115, H216, K217, L218, and K219 of SEQ ID NO: 1.

5. The recombinant polypeptide of any one of claims 1 to 4, wherein the one or more amino acid substitution is at a position corresponding to an amino acid residue selected from the group consisting of W37, P39, D40, E81, F82, K106, K217, and K219 of SEQ ID NO: 1.

6. The recombinant polypeptide of any one of claims 1 to 5, comprising an amino acid sequence having at least 70%, 80%, 90%, 95%, 99%, or 100% sequence identity to SEQ ID NO: 1, and further comprising the amino acid substitutions corresponding to the following amino acid substitutions:
a) W37A;
b) P39A;
c) D40A;
d) E81A;
e) F82A;
f) K106;
g) D109A;
h) K217A;
i) K219A;
j) E81A/F82A;
k) W37A/E81A/F82A;
l) E81A/F82A/K106A;
m) E81A/F82A/K106A/K219A;
n) E81A/F82A/K106A/K217A;
o) 81A/F82A/K106A/E108A/D115A;
p) E81F/F82A;
q) E81K/F82A;
r) E81L/F82A;
s) E81H/F82A;
t) E81 S/F82A;
u) E81A/F82A/K106N;
v) E81A/F82A/K106Q;
w) E81A/F82A/K106T;
x) E81A/F82A/K106R; or
(y) P39A/D40A/E81A/F82A.

7. The recombinant polypeptide of any one of claims 1 to 6, comprising an amino acid sequence selected from the group consisting of SEQ ID NOS: 3-8 and 13-16.

8. A recombinant polypeptide comprising:
an amino acid sequence having one or more 70%, 80%, 90%, 95%, 99%, or 100% sequence identity to an interleukin 12 subunit p40 (IL-12p40) polypeptide having the amino acid sequence of SEQ ID NO: 2;
and further comprising one or more amino acid substitution at a position corresponding to an amino acid residue selected from the group consisting of X37, X39, X40, X41, X80, X81, X82, X106, X108, X115, X216, X217, X218, and X219 of SEQ ID NO: 2.

9. The recombinant polypeptide of claim 8, wherein the one or more amino acid substitution is at a position corresponding to an amino acid residue selected from the group consisting of X37, X39, X40, X81, X82, X106, X217, and X219 of SEQ ID NO: 2.

10. The recombinant polypeptide of any one of claims 8 to 9, wherein the one or more amino acid substitution is independently selected from the group consisting of an alanine (A) substitution, an arginine (R) substitution, an asparagine (N) substitution, an aspartic acid (D) substitution, a leucine (L) substitution, a lysine (K) substitution, a phenylalanine (F) substitution, a lysine substitution, a glutamine (Q) substitution, a glutamic acid (E) substitution, a serine (S) substitution, and a threonine (T) substitution.

11. The recombinant polypeptide of any one of claims 8 to 10, wherein the one or more amino acid substitution is at a position corresponding to an amino acid residue selected from the group consisting of W37, P39, D40, A41, K80, E81, F82, K106, E108, D115, H216, K217, L218, and E219 of SEQ ID NO: 2.

12. The recombinant polypeptide of any one of claims 8 to 11, wherein the one or more amino acid substitution is at a position corresponding to an amino acid residue selected from the group consisting of P39, D40, E81, F82, K106, K217, and E219 of SEQ ID NO: 2.

13. The recombinant polypeptide of any one of claims 8 to 12, comprising an amino acid sequence having one or more 70%, 80%, 90%, 95%, 99%, or 100% sequence identity to SEQ ID NO: 2, and further comprising the amino acid substitutions corresponding to the following amino acid substitutions:
a) W37A;
b) P39A;
c) D40A;
d) E81A;
e) F82A;
f) K106;
g) D109A;
h) K217A;
i) E219A;
j) E81A/F82A;
k) W37A/E81A/F82A;
l) E81A/F82A/K106A;
m) E81A/F82A/K106A/K217A;
n) E81F/F82A;
o) E81K/F82A;
p) E81L/F82A;
q) E81H/F82A;
r) E81 S/F82A;
s) E81A/F82A/K106N;
t) E81A/F82A/K106Q;
u) E81A/F82A/K106T;
v) E81A/F82A/K106R; or
w) P39A/D40A/E81A/F82A.

14. The recombinant polypeptide of any one of claims 8 to 13, comprising an amino acid sequence selected from the group consisting of SEQ ID NOS: 9-11 and 17-25.

15. The recombinant polypeptide of any one of claims 1 to 14, wherein the recombinant polypeptide has an altered binding affinity for interleukin-12 receptor, subunit beta 1 (IL-12Rβ1) compared to binding affinity of a reference polypeptide lacking the one or more amino acid substitution.

16. The recombinant polypeptide of claim 15, wherein the recombinant polypeptide has a reduced binding affinity for IL-12Rβ1 compared to binding affinity of a reference polypeptide lacking the one or more amino acid substitution.

17. The recombinant polypeptide of any one of claims 15 to 15, wherein the recombinant polypeptide has binding affinity for IL-12Rβ1 reduced by about 10% to about 100% compared to binding affinity of a reference polypeptide lacking the one or more amino acid substitution, as determined by surface plasmon resonance (SPR).

18. The recombinant polypeptide of any one of claims 15 to 17, wherein the recombinant polypeptide, when combined with an interleukin 12 subunit p35 (IL-12p35) polypeptide, has a reduced capability to stimulate STAT4 signaling compared to a reference polypeptide lacking the one or more amino acid substitution.

19. The recombinant polypeptide of any one of claims 15 to 18, wherein the recombinant polypeptide, when combined with an interleukin 23 subunit p19 (IL-23p19) polypeptide, has a reduced capability to stimulate STAT3 signaling compared to a reference polypeptide lacking the one or more amino acid substitution.

20. The recombinant polypeptide of any one of claims 18 to 19, wherein the STAT3 signaling and/or STAT4 signaling is determined by an assay selected from the group consisting of by a gene expression assay, a phospho-flow signaling assay, and an enzyme-linked immunosorbent assay (ELISA).

21. The recombinant polypeptide of any one of claims 15 to 20, wherein the one or more amino acid substitution results in a cell-type biased signaling of the downstream signal transduction mediated through interleukin-12 (IL-12) and/or interleukin-23 (IL-23) compared to a reference polypeptide lacking the one or more amino acid substitution.

22. The recombinant polypeptide of claim 21, wherein the cell-type biased signaling comprises a reduced capability of the recombinant polypeptide to stimulate IL-12-mediated signaling in natural killer (NK) cells.

23. The recombinant polypeptide of any one of claims 21 to 22, wherein the cell-type biased signaling comprises a substantially unaltered capability of the recombinant polypeptide to stimulate IL-12 signaling in CD8+ T cells.

24. The recombinant polypeptide of any one of claims 21 to 23, wherein the one or more amino acid substitution results in a reduced capability of the recombinant polypeptide to stimulate IL-12 signaling in NK cells while substantially retains its capability to stimulate IL-12 signaling in CD8+ T cells.

25. A recombinant nucleic acid molecule comprising a nucleic acid sequence encoding a polypeptide that comprises an amino acid sequence having at least 90% sequence identity to the amino acid sequence of the polypeptide of any one of claims 1 to 24.

26. The nucleic acid molecule of claim 25, wherein the nucleic acid sequence is operably linked to a heterologous nucleic acid sequence.

27. The nucleic acid molecule of any one of claims 25 to 26, wherein the nucleic acid molecule is further defined as an expression cassette or an expression vector.

28. A recombinant cell comprising:
a) a recombinant polypeptide according to any one of claims 1 to 24; and/or
b) a recombinant nucleic acid according to any one of claims 25 to 27.

29. The recombinant cell of claim 28, wherein the recombinant cell is a eukaryotic cell.

30. The recombinant cell of claim 29, wherein the eukaryotic cell is a mammalian cell

31. A cell culture comprising at least one recombinant cell of any one of claims 28 to 30 and a culture medium.

32. A method for producing a recombinant polypeptide, comprising:
a) providing one or more recombinant cells of any one of claims 28 to 30; and
b) culturing the one or more recombinant cells in a culture medium such that the cells produce the polypeptide encoded by the recombinant nucleic acid molecule.

33. The method of claim 32, further comprising isolating and/or purifying the produced polypeptide.

34. The method of any one of claims 32 to 33, further comprising structurally modifying the produced polypeptide to increase half-life.

35. The method of claim 34, wherein said modification comprises one or more alterations selected from the group consisting of fusion to a human Fc antibody fragment, fusion to albumin, and PEGylation.

36. A recombinant polypeptide produced by the method of any one of claims 32 to 35.

37. A pharmaceutical composition comprising:
a) a recombinant polypeptide according to any one of claims 1-24 and 36;
b) a recombinant nucleic acid according to any one of claims 25 to 27;
c) a recombinant cell according to any one of claims to 28 to 30; and/or
c) a pharmaceutically acceptable carrier.

38. The pharmaceutical composition of claim 37, wherein the composition comprises a recombinant polypeptide according to any one of claims 1-24 and 36, and a pharmaceutically acceptable carrier.

39. The pharmaceutical composition of claim 37, wherein the composition comprises a recombinant nucleic acid according to any one of claims 25 to 27, and a pharmaceutically acceptable carrier.

40. A method for modulating IL-12-mediated signal transduction in a subject, the method comprising administering to the subject a composition comprising:
a) a recombinant IL-12p40 polypeptide according to any one of claims 1-24 and 36;
b) a recombinant nucleic acid according to any one of claims 25 to 27;
c) a recombinant cell according to any one of claims to 28 to 30; and/or
d) a pharmaceutically composition according to claims 37 to 39.

41. The method of claim 40, further comprising administering to the subject an IL-12p35 polypeptide, or nucleic acid encoding the IL-12p35 polypeptide.

42. A method for modulating IL-23-mediated signal transduction in a subject, the method comprising administering to the subject a composition comprising:
a) a recombinant IL-12p40 polypeptide according to any one of claims 1-24 and 36;
b) a recombinant nucleic acid according to any one of claims 25 to 27;
c) a recombinant cell according to any one of claims to 28 to 30; and/or
d) a pharmaceutically composition according to claims 37 to 39.

43. The method of claim 42, further comprising administering to the subject an IL-12p35 polypeptide, or nucleic acid encoding the IL-23p19 polypeptide.

44. A method for the treatment of a condition in a subject in need thereof, the method comprising administering to the subject a composition comprising:
a) a recombinant IL-12p40 polypeptide according to any one of claims 1-24 and 36;
b) a recombinant nucleic acid according to any one of claims 25 to 27;
c) a recombinant cell according to any one of claims to 28 to 30; and/or
d) a pharmaceutically composition according to claims 37 to 39.

45. The method of claim 44, further comprising administering to the subject:
a) an IL-12p35 polypeptide;
b) an IL-23p19 polypeptide; and/or
c) nucleic acid encoding (a) or (b) above.

46. The method of any one of claims 40 to 45, wherein the recombinant polypeptide has an altered binding affinity for interleukin-12 receptor, beta 1 (IL-12Rβ1) compared to binding affinity of a reference polypeptide lacking the one or more amino acid substitution.

47. The method of any one of claims 40 to 46, wherein the recombinant polypeptide has a reduced binding affinity for IL-12Rβ1 compared to binding affinity of a reference polypeptide lacking the one or more amino acid substitution.

48. The method of any one of claims 40 to 47, wherein the recombinant polypeptide has binding affinity for IL-12Rβ1 reduced by about 10% to about 100% compared to binding affinity of a reference polypeptide lacking the one or more amino acid substitution, as determined by surface plasmon resonance (SPR).

49. The method of any one of claims 40 to 48, wherein the reduced binding affinity of the recombinant polypeptide to IL-12Rβ1 receptor results in a reduction in STAT4-mediated signaling compared to a reference polypeptide lacking the one or more amino acid substitution.

50. The method of any one of claims 40 to 49, wherein the reduced binding affinity of the recombinant polypeptide to IL-12Rβ1 receptor results in a reduction in STAT3-mediated signaling compared to a reference polypeptide lacking the one or more amino acid substitution.

51. The method of any one of claims 49 to 50, wherein the STAT3 signaling and/or STAT4 signaling is determined by an assay selected from the group consisting of by a gene expression assay, a phospho-flow signaling assay, and an enzyme-linked immunosorbent assay (ELISA).

52. The method of any one of claims 40 to 51, wherein the administered composition results in a cell-type biased signaling of the downstream signal transduction mediated by interleukin-12 (IL-12) and/or by interleukin-23 (IL-23) compared to a reference polypeptide lacking the one or more amino acid substitution.

53. The method of claim 52, wherein the cell-type biased signaling comprises a reduced capability of the recombinant polypeptide to stimulate IL-12-mediated signaling in NK cells.

54. The method of any one of claims 52 to 53, wherein the cell-type biased signaling comprises a substantially unaltered capability of the recombinant polypeptide to stimulate IL-12 signaling in CD8+ T cells.

55. The method of any one of claims 40 to 54, wherein the administered composition results in a reduced capability of the recombinant polypeptide to stimulate IL-12 signaling in NK cells while substantially retains its capability to stimulate IL-12 signaling in CD8+ T cells.

56. The methods of claim 55, wherein the administered composition substantially retains the recombinant polypeptide's capability to stimulate expression of interferon gamma (INFγ) in CD8+ T cells.

57. The method of any one of claims 40 to 56, wherein the administered composition enhances antitumor immunity in a tumor microenvironment.

58. The method of any one of claims 40 to 57, wherein the subject is a mammal.

59. The method of claim 58, wherein the mammal is a human.

60. The method of any one of claims 40 to 59, wherein the subject has or is suspected of having a condition associated with IL-12p40 mediated signaling.

61. The method of claim 60, wherein the IL-12p40 mediated signaling is IL-12 mediated signaling or IL-23 mediated signaling.

62. The method of claim 60, wherein the condition is a cancer, an immune disease, or a chronic infection.

63. The method of claim 62, wherein the immune disease is an autoimmune disease.

64. The method of claim 63, wherein the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, moderate to severe plaque psoriasis, psoriatic arthritis, Crohn's disease, ulcerative colitis, and graft vs. host disease.

65. The method of claim 62, wherein the condition is a cancer selected from the group consisting of an acute myeloma leukemia, an anaplastic lymphoma, an astrocytoma, a B-cell cancer, a breast cancer, a colon cancer, an ependymoma, an esophageal cancer, a glioblastoma, a glioma, a leiomyosarcoma, a liposarcoma, a liver cancer, a lung cancer, a mantle cell lymphoma, a melanoma, a neuroblastoma, a non-small cell lung cancer, an oligodendroglioma, an ovarian cancer, a pancreatic cancer, a peripheral T-cell lymphoma, a renal cancer, a sarcoma, a stomach cancer, a carcinoma, a mesothelioma, and a sarcoma.

66. The method of any one of claims 40 to 65, wherein the composition is administered to the subject individually as a first therapy or in combination with a second therapy.

67. The method of claim 66, wherein the second therapy is selected from the group consisting of chemotherapy, radiotherapy, immunotherapy, hormonal therapy, toxin therapy, or surgery.

68. The method of any one of claims 66 to 67, wherein the first therapy and the second therapy are administered concomitantly.

69. The method of any one of claims 66 to 68, wherein the first therapy is administered at the same time as the second therapy.

70. The method of any one of claims 66 to 68, wherein the first therapy and the second therapy are administered sequentially.

71. The method of claim 70, wherein the first therapy is administered before the second therapy.

72. The method of claim 70, wherein the first therapy is administered after the second therapy.

73. The method of claim 70, wherein the first therapy is administered before and/or after the second therapy.

74. The method of any one of claims 66 to 73, wherein the first therapy and the second therapy are administered in rotation.

75. The method of any one of claims 66 to 67, wherein the first therapy and the second therapy are administered together in a single formulation.

76. A kit for modulating IL-12p40 mediated signal transduction, modulating IL-12p40-mediated signal transduction, or treating a condition in a subject in need thereof, the system comprising:
a) a recombinant polypeptide according to any one of claims 1-24 and 36;
b) a recombinant nucleic acid according to any one of claims 25 to 27;
c) a recombinant cell according to any one of claims 28 to 31; and/or
d) a pharmaceutical composition according to any one of claims 37 to 39; and
instructions for performing the method of any one of claims 40 to 75.

77. Use of the following for the manufacture of a medicament for the treatment and/or prevention of a condition associated with a health condition associated with a perturbation in IL-12-p40 mediated signal transduction:
a) a recombinant polypeptide according to any one of claims 1-24 and 36;
b) a recombinant nucleic acid according to any one of claims 25 to 27;
c) a recombinant cell according to any one of claims 28 to 31; and/or
d) a pharmaceutical composition according to any one of claims 37 to 39.
